(19) United States
(12) Patent Application Publication
(10) Pub. No.: US 2016/0168609 A1
(43) Pub. Date: Jun. 16, 2016

(54) ENHANCED CELLULOSE DEGRADATION

(71) Applicant: THE REGENTS OF THE UNIVERSITY OF CALIFORNIA, Oakland, CA (US)

(72) Inventors: Michael A. MARLETTA, La Jolla, CA (US); James H. DOUDNA CATE, Berkeley, CA (US); William T. BEESON, IV, Indianapolis, IN (US); Christopher M. PHILLIPS, San Diego, CA (US)

(73) Assignee: THE REGENTS OF THE UNIVERSITY OF CALIFORNIA, Oakland, CA (US)

(21) Appl. No.: 14/941,492

(22) Filed: Nov. 13, 2015

Related U.S. Application Data
(62) Division of application No. 14/008,525, filed on Nov. 18, 2013, now abandoned.
(60) Provisional application No. 61/510,463, filed on Jul. 21, 2011.
(60) Provisional application No. 61/471,627, filed on Apr. 4, 2011.

(51) Int. Cl. C12P 19/14; C12P 19/02

(52) U.S. Cl. CPC C12P 19/14; C12P 19/02

(57) ABSTRACT

The present disclosure provides compositions and methods related to the degradation of cellulose and cellulose-containing materials. CDH-heme domain polypeptides and GH61 polypeptides and related polynucleotides and compositions are provided herein. Additionally, methods related to CDH-heme domain polypeptides, GH61 polypeptides, and related polynucleotides and compositions, are provided herein

### CROSS-REFERENCE TO RELATED APPLICATIONS

This application is a Divisional of U.S. patent application Ser. No. 14/008,525, filed Apr. 4, 2012, which is a U.S. National Phase patent application of PCT/US2012/032188, filed Apr. 4, 2012, which claims the benefit of U.S. Provisional Patent Application No. 61/471,627, filed Apr. 4, 2011, and U.S. Provisional Application No. 61/510,463, filed Jul. 21, 2011. Each of the above-referenced applications are hereby incorporated by reference in their entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 677792001410SEQLIST.txt, date recorded: Nov. 13, 2015, size: 194 KB).

### FIELD

The present disclosure relates to methods and compositions for degradation of cellulose and cellulose-containing materials. In particular, the disclosure relates polypeptides, polynucleotides, and compositions related to degradation of cellulose, and methods of use thereof.

### BACKGROUND

Biofuels are under intensive investigation due to the increasing concerns about energy security, sustainability, and global climate change. Bioconversion of plant-based materials into biofuels is regarded as an attractive alternative to chemical production of fossil fuels.

Cellulose, a major component of plants and one of the most abundant organic compounds on earth, is a polysaccharide composed of long chains of β(1-4) linked D-glucose molecules. Due to its sugar-based composition, cellulose is a rich potential source material for the production of biofuels and other sugar-derived products. For example, sugars may be fermented into biofuels such as ethanol. In order for the sugars within cellulose to be used for the production of biofuels, the cellulose must be broken down into smaller molecules.

Cellulose may be degraded by chemical or enzymatic means. Enzymes that hydrolyze cellulose are referred to as “cellulases” and include, for example, endoglucanases, exoglucanases, and beta-glucosidases.

Although techniques exist for the break down of cellulose, current techniques are relatively inefficient and expensive, which has limited the implementation of cellulose-based technologies. Accordingly, there is great interest in the development of reagents and techniques for improving the efficiency of cellulose degradation. One approach to improving the efficiency of cellulose degradation is to improve the catalytic activity of cellulase enzymes. An alternative approach (which may be used in conjunction with improving the catalytic activity of cellulases) is to develop compositions that can be used with cellulases to increase the degradation of cellulose, and to develop methods of their use.

### BRIEF SUMMARY

Polypeptides, polynucleotides, compositions, and methods for increasing the degradation of cellulose are disclosed herein. These polypeptides, polynucleotides, compositions, and methods provide a dramatic improvement in cellulose degradation over prior polypeptides, polynucleotides, compositions and methods.

A non-naturally occurring polypeptide, having a first domain and a second domain, wherein the first domain contains a CDH-heme domain and the second domain contains a cellulose binding module (CBM) is disclosed herein. These polypeptides are more effective at degrading cellulose than CDH-heme domain containing-polypeptides which lack a CBM.

A non-naturally occurring polypeptide lacking a dehydrogenase domain but having CDH-heme and CBM domains is also disclosed. Cellulase reactions utilizing such polypeptides produce fewer reactive oxygen species thereby reducing oxidative damage. Such oxidative damage can reduce cellulase enzyme activity, chemically alter enzyme substrates or products, and/or generate undesirable side products.

Compositions containing a recombinant GH61 polypeptide and a recombinant CDH-heme domain polypeptide containing a CBM are disclosed. These compositions may include various GH61 polypeptides and CDH-heme domain polypeptides provided herein. These compositions may be included with mixtures that contain cellulases and cellulose-containing material to increase the degradation of cellulose-containing material.

Various recombinant GH61 polypeptides are also disclosed. These polypeptides may be provided with mixtures that contain cellulases and cellulose-containing material to increase degradation of the cellulose-containing material.

Recombinant GH61 polypeptides that are bound to a copper atom are described herein. These polypeptides are more effective at degrading cellulose than otherwise equivalent GH61 polypeptides which are not bound to a copper atom

Also disclosed are various recombinant CDH-heme domain polypeptides containing a CBM. In some aspects, these polypeptides have higher activity under aerobic conditions than under anaerobic conditions. As such, providing supplemental oxygen to the reaction can improve the reaction. Such oxygen can be provided by bubbling air in the reaction or other standard means.

A non-naturally occurring polypeptide, having a first domain and a second domain, wherein the first domain contains a CDH-heme domain and the second domain contains a cellulose binding module (CBM) is also disclosed. In one format, the polypeptide will not include a dehydrogenase domain. Also disclosed are the recombinant polynucleotides encoding such polypeptides.

A non-naturally occurring polypeptide having first, second and third domains is also disclosed. The first domain may contain a CDH-heme domain, the second domain may contain a CBM domain, and the third domain may contain a dehydrogenase domain. Also disclosed are the recombinant polynucleotides encoding such polypeptides.

A composition containing a recombinant GH61 polypeptide and a recombinant CDH-heme domain polypeptide containing a CBM is also disclosed. The recombinant GH61 polypeptide may contain the motif H-X₍₄₋₈₎-Q-X-Y. In another format, the GH61 polypeptide may contain a polypeptide of the NCU02240/NCU01050 clade. In another format, the recombinant GH61 polypeptide contains SEQ ID NO: 24 (NCU02240) or 30 (NCU01050). In another format, the GH61 polypeptide contains SEQ ID NO: 26 (NCU07898), 28 (NCU08760), SEQ ID NO: 90 (NCU00836). Any of these compositions may further contain one or more cellulases.

A composition containing a recombinant GH61 polypeptide and a recombinant CDH-heme domain polypeptide containing a CBM is disclosed where the CBM contains SEQ ID NOs: 32 (N. crassa CDH-1) or 46 (M. thermophila CDH-1). The composition may further contain one or more cellulases.

A composition containing: A) a recombinant GH61 polypeptide, and B) a recombinant non-naturally occurring polypeptide containing a CDH-heme domain and a CBM domain is provided. The non-naturally occurring polypeptide optionally contains a dehydrogenase domain. The composition may further contain one or more cellulases.

Also provided is a composition containing: A) a first polypeptide that includes a CDH-heme domain and B) second polypeptide that contains a CBM, where the first and second polypeptides stably interact but are not covalently linked. In one format, the first polypeptide and the second polypeptide interact through a leucine zipper motif. In one format, the CDH-heme domain contains an amino acid sequence selected from SEQ ID NOs: 70 (N. crassa CDH-1 heme domain); 76 (N. crassa CDH-2 heme domain); 80 (M. thermophila CDH-1 heme domain); and 86 (M. thermophila CDH-2 heme domain), and the CBM contains an amino acid sequence of SEQ ID NOs: 74 (N. crassa CDH-1 CBM domain) or 84 (M. thermophila CDH-1 CBM domain). In another format, any of these compositions are provided with a GH61 polypeptide. In another format, any of these compositions may further contain one or more cellulases.

A composition containing A) a recombinant GH61 polypeptide and B) a recombinant CDH-heme domain polypeptide containing a CBM, where the CDH-heme domain contains an amino acid sequence selected from SEQ ID NOs: 70 (N. crassa CDH-1 heme domain); 76 (N. crassa CDH-2 heme domain); 80 (M. thermophila CDH-1 heme domain); and 86 (M. thermophila CDH-2 heme domain), and where the CBM contains an amino acid sequence of SEQ ID NOs: 74 (N. crassa CDH-1 CBM domain) or 84 (M. thermophila CDH-1 CBM domain) is described herein. In one format, the recombinant GH61 polypeptide of the composition contains a polypeptide of the NCU02240/NCU01050 clade. In one format, the recombinant GH61 polypeptide of the composition contains SEQ ID NO: 24 (NCU02240) or 30 (NCU01050). In another format, the recombinant GH61 polypeptide of the composition contains SEQ ID NO: 26 (NCU07898) or 28 (NCU08760). In another format, the recombinant CDH-heme domain polypeptide containing a CBM of the composition contains SEQ ID NOs: 32 (N. crassa CDH-1) or 46 (M. thermophila CDH-1). Any of these compositions may further contain one or more cellulases.

A composition containing A) a recombinant GH61 polypeptide and B) a non-naturally occurring CDH-heme domain polypeptide containing a CBM and lacking a dehydrogenase domain, where the CDH-heme domain contains an amino acid sequence selected from SEQ ID NOs: 70 (N. crassa CDH-1 heme domain); 76 (N. crassa CDH-2 heme domain); 80 (M. thermophila CDH-1 heme domain); and 86 (M. thermophila CDH-2 heme domain), and where the CBM contains an amino acid sequence of SEQ ID NOs: 74 (N. crassa CDH-1 CBM domain) or 84 (M. thermophila CDH-1 CBM domain) is described herein. The composition may further contain one or more cellulases.

A composition containing A) a recombinant GH61 polypeptide and B) a non-naturally occurring CDH-heme domain polypeptide containing a CBM and containing a dehydrogenase domain, where the CDH-heme domain contains an amino acid sequence selected from SEQ ID NOs: 70 (N. crassa CDH-1 heme domain); 76 (N. crassa CDH-2 heme domain); 80 (M. thermophila CDH-1 heme domain); and 86 (M. thermophila CDH-2 heme domain), and where the CBM contains an amino acid sequence of SEQ ID NOs: 74 (N. crassa CDH-1 CBM domain) or 84 (M. thermophila CDH-1 CBM domain) is also described herein. The composition may further contain one or more cellulases.

A composition containing A) a recombinant GH61 polypeptide, B) a recombinant CDH-heme domain polypeptide containing a CBM, and C) one or more cellulases is also provided herein. In one format, the recombinant GH61 polypeptide of the composition contains a polypeptide of the NCU02240/NCU01050 clade. In one format, the recombinant GH61 polypeptide of the composition contains SEQ ID NO: 24 (NCU02240) or 30 (NCU01050). In one format, the recombinant GH61 polypeptide of the composition contains SEQ ID NO: 26 (NCU07898) or 28 (NCU08760). In another format, the recombinant CDH-heme domain polypeptide containing a CBM of the composition contains SEQ ID NOs: 32 (N. crassa CDH-1) or 46 (M. thermophila CDH-1). In another format, the recombinant CDH-heme domain polypeptide containing a CBM is a non-naturally occurring polypeptide

A host cell containing recombinant polynucleotides encoding a GH61 polypeptide and a CDH-heme domain polypeptide containing a CBM is also provided herein. In one format, the polynucleotide encoding a CDH-heme domain polypeptide containing a CBM encodes a non-naturally occurring polypeptide.

A method of degrading cellulose, the method including contacting the cellulose with one or more cellulases and a composition containing a recombinant GH61 polypeptide and a recombinant CDH-heme domain polypeptide containing a CBM, to yield degraded cellulose, is also provided. In one format, the recombinant GH61 polypeptide contains the motif H-X₍₄₋₈₎-Q-X-Y. In one format, the recombinant GH61 polypeptide of the method contains a polypeptide of the NCU02240/NCU01050 clade. In one format, the recombinant GH61 polypeptide of the method contains SEQ ID NO: 24 (NCU02240) or 30 (NCU01050). In one format, the recombinant GH61 polypeptide of the method contains SEQ ID NO: 26 (NCU07898), 28 (NCU08760), or SEQ ID NO: 90 (NCU00836). In another format, the recombinant CDH-heme domain polypeptide containing a CBM of the method contains SEQ ID NOs: 32 (N. crassa CDH-1) or 46 (M. thermophila CDH-1). In another format, the recombinant CDH-heme domain polypeptide containing a CBM of the method is a non-naturally occurring polypeptide, containing a first domain containing a CDH-heme domain and a second domain containing a CBM, and not including a dehydrogenase domain. In another format, the recombinant CDH-heme domain polypeptide containing a CBM of the method is a non-naturally occurring polypeptide, containing a first domain containing a CDH-heme domain, a second domain containing a CBM, and a third domain including a dehydrogenase domain. In any of the above methods, the cellulose may be in biomass. In such methods, the method results in degraded biomass. In methods involving biomass, the biomass may be subject to a preprocessing step.

A method of degrading cellulose, the method including contacting the cellulose with one or more cellulases and a composition containing a first polypeptide containing a CDH-heme domain and second polypeptide containing a CBM, where the first polypeptide and second polypeptide stably interact but are not covalently linked, is provided. In one format of the method, the first polypeptide and second polypeptide interact through a leucine zipper motif. In another format of the method, a GH61 polypeptide may be included with the cellulases and the composition. In any of the above methods, the cellulose may be in biomass. In such methods, the method results in degraded biomass. In methods involving biomass, the biomass may be subject to a preprocessing step.

Also provided herein is a method of converting biomass to fermentation product, the method including contacting the biomass with one or more cellulases and a composition containing a recombinant GH61 polypeptide and a recombinant CDH-heme domain polypeptide containing a CBM, to yield a sugar solution; and culturing the sugar solution with a fermentative microorganism under conditions sufficient to produce a fermentation product. In this method, the biomass may be subjected to a preprocessing step. In one format, the recombinant GH61 polypeptide of the method is a polypeptide of the NCU02240/NCU01050 clade. In one format, the recombinant GH61 polypeptide of the method contains SEQ ID NO: 24 (NCU02240) or 30 (NCU01050). In another format, the recombinant GH61 polypeptide of the method contains SEQ ID NO: 26 (NCU07898), 28 (NCU08760), or SEQ ID NO: 90 (NCU00836). In one format, the recombinant CDH-heme domain polypeptide containing a CBM of the method contains SEQ ID NOs: 32 (N. crassa CDH-1) or 46 (M. thermophila CDH-1). In another format, the recombinant CDH-heme domain polypeptide containing a CBM of the method is a non-naturally occurring polypeptide, containing a first domain that includes a CDH-heme domain and a second domain that includes a CBM, and that does not contain a dehydrogenase domain. In another format, the recombinant CDH-heme domain polypeptide containing a CBM of the method is a non-naturally occurring polypeptide, containing a first domain that includes a CDH-heme domain, a second domain that includes a CBM, and a third domain that includes a dehydrogenase domain.

Further provided herein is a method of converting biomass to fermentation product, the method including contacting the biomass with one or more cellulases and a composition containing a first polypeptide containing a CDH-heme domain and second polypeptide containing a CBM, wherein the first polypeptide and the second polypeptide stably interact but are not covalently linked, to yield a sugar solution; and culturing the sugar solution with a fermentative microorganism under conditions sufficient to produce a fermentation product. In this method, the biomass may be subjected to a preprocessing step. In one format, the first polypeptide and the second polypeptide interact through a leucine zipper motif. In another format of the method, a GH61 polypeptide may be included with the cellulases and the composition.

A method of increasing the rate of degradation of cellulose in a mixture containing cellulose and cellulases is provided herein, the method including contacting the mixture containing cellulose and cellulases with a composition containing a recombinant GH61 polypeptide and a recombinant CDH-heme domain polypeptide containing a CBM. In one format, the recombinant GH61 polypeptide of the method is a polypeptide of the NCU02240/NCU01050 clade. In one format, the recombinant GH61 polypeptide of the method contains SEQ ID NO: 24 (NCU02240) or 30 (NCU01050). In another format, the recombinant GH61 polypeptide of the method contains SEQ ID NO: 26 (NCU07898), 28 (NCU08760), or SEQ ID NO: 90 (NCU00836). In one format, the recombinant CDH-heme domain polypeptide containing a CBM of the method contains SEQ ID NOs: 32 (N. crassa CDH-1) or 46 (M. thermophila CDH-1). In another format, the recombinant CDH-heme domain polypeptide containing a CBM of the method is a non-naturally occurring polypeptide, containing a first domain that includes a CDH-heme domain and a second domain that includes a CBM, and that does not contain a dehydrogenase domain. In another format, the recombinant CDH-heme domain polypeptide containing a CBM of the method is a non-naturally occurring polypeptide, containing a first domain that includes a CDH-heme domain, a second domain that includes a CBM, and a third domain that includes a dehydrogenase domain.

A method of increasing the rate of degradation of cellulose in a mixture containing cellulose and cellulases is provided herein, the method including contacting the mixture containing cellulose and cellulases with a composition containing a first polypeptide containing a CDH-heme domain and second polypeptide containing a CBM, wherein the first polypeptide and the second polypeptide stably interact but are not covalently linked. In one format, the first polypeptide and the second polypeptide interact through a leucine zipper motif. In another format of the method, a GH61 polypeptide may be included with the cellulases and the composition.

A method of reducing the viscosity of a pre-treated biomass mixture is provided herein, the method including contacting the mixture with cellulases and a composition containing a recombinant GH61 polypeptide and a recombinant CDH-heme domain polypeptide containing a CBM, to yield a pre-treated biomass mixture having reduced viscosity. In one format, the recombinant GH61 polypeptide of the method is a polypeptide of the NCU02240/NCU01050 clade. In one format, the recombinant GH61 polypeptide of the method contains SEQ ID NO: 24 (NCU02240) or 30 (NCU01050). In another format, the recombinant GH61 polypeptide of the method contains SEQ ID NO: 26 (NCU07898), 28 (NCU08760), or SEQ ID NO: 90 (NCU00836). In one format, the recombinant CDH-heme domain polypeptide containing a CBM of the method contains SEQ ID NOs: 32 (N. crassa CDH-1) or 46 (M. thermophila CDH-1). In another format, the recombinant CDH-heme domain polypeptide containing a CBM of the method is a non-naturally occurring polypeptide, containing a first domain that includes a CDH-heme domain and a second domain that includes a CBM, and that does not contain a dehydrogenase domain. In another format, the recombinant CDH-heme domain polypeptide containing a CBM of the method is a non-naturally occurring polypeptide, containing a first domain that includes a CDH-heme domain, a second domain that includes a CBM, and a third domain that includes a dehydrogenase domain.

Also disclosed herein is a method of producing glucose and 4-keto glucose molecules, the method including contacting cellulose with a recombinant GH61 polypeptide and a recombinant CDH-heme domain polypeptide containing a CBM, wherein the recombinant GH61 polypeptide is bound to a copper atom. In one format, the recombinant GH61 polypeptide of the method is a polypeptide of the NCU02240/NCU01050 clade. In one format, the recombinant GH61 polypeptide of the method contains SEQ ID NO: 24 (NCU02240) or 30 (NCU01050). In another format, the recombinant GH61 polypeptide of the method contains SEQ ID NO: 26 (NCU07898), 28 (NCU08760) or SEQ ID NO: 90 (NCU00836).

Also disclosed herein is a method of cleaving a 1-4 glycosidic bond in a cellulose polymer, the method including contacting cellulose with a recombinant GH61 polypeptide and a recombinant CDH-heme domain polypeptide containing a CBM, wherein the recombinant GH61 polypeptide is bound to a copper atom. In one format, the recombinant GH61 polypeptide of the method is a polypeptide of the NCU02240/NCU01050 clade. In one format, the recombinant GH61 polypeptide of the method contains SEQ ID NO: 24 (NCU02240) or 30 (NCU01050). In another format, the recombinant GH61 polypeptide of the method contains SEQ ID NO: 26 (NCU07898), 28 (NCU08760) or SEQ ID NO: 90 (NCU00836).

Also disclosed herein is a method of cleaving the C—H bond at the carbon 4 position of a glucose molecule, the method including contacting cellulose with a recombinant GH61 polypeptide and a recombinant CDH-heme domain polypeptide containing a CBM, wherein the recombinant GH61 polypeptide is bound to a copper atom. In one format, the recombinant GH61 polypeptide of the method is a polypeptide of the NCU02240/NCU01050 clade. In one format, the recombinant GH61 polypeptide of the method contains SEQ ID NO: 24 (NCU02240) or 30 (NCU01050). In another format, the recombinant GH61 polypeptide of the method contains SEQ ID NO: 26 (NCU07898), 28 (NCU08760) or SEQ ID NO: 90 (NCU00836).

In some aspects, at least 50% of the GH61 polypeptides in a method or composition provided above are bound to a copper atom. In some aspects, at least 90% of the GH61 polypeptides in a method or composition provided above are bound to a copper atom.

Also disclosed herein is a composition containing multiple recombinant GH61 polypeptides, wherein at least 50%, 60%, 70%, 80%, 90%, 95%, 98%, or 99% of the GH61 polypeptides are bound to a copper atom. In one format, the recombinant GH61 polypeptides of the composition are polypeptides of the NCU02240/NCU01050 clade. In one format, the recombinant GH61 polypeptides of the composition contain SEQ ID NO: 24 (NCU02240) or 30 (NCU01050). In another format, the recombinant GH61 polypeptides of the composition contain SEQ ID NO: 26 (NCU07898), 28 (NCU08760) or SEQ ID NO: 90 (NCU00836).

A method of producing a GH61 polypeptide is provided herein, the method including culturing a cell containing a recombinant polynucleotide encoding a GH61 polypeptide in a media that contains 0.1-1000 μM copper, and subjecting the cell to conditions sufficient to produce GH61 polypeptide from the recombinant polynucleotide encoding the GH61 polypeptide. In one format of the method, the media contains 100-800 μM copper.

Also disclosed herein is a method of degrading cellulose, the method including contacting the cellulose with one or more one or more cellulases, a recombinant CDH-heme domain protein containing a CBM, and a recombinant GH61 polypeptide, wherein the recombinant GH61 polypeptide includes: i) a polypeptide of the NCU2240/NCU01050 clade or ii) an amino acid sequence selected from the group consisting of: SEQ ID NO: 90 (NCU00836), SEQ ID NO: 26 (NCU07898), or SEQ ID NO: 28 (NCU08760), in a reaction mixture that has a concentration of copper between 0.1-500 μM. In one format of the method, the reaction mixture has a concentration of copper between 1-50 μM.

A method of increasing the rate of degradation of cellulose in a mixture containing cellulose, cellulases, a CDH-heme domain polypeptide containing a CMB, and a GH61 polypeptide, the method including providing 1-50 μM copper in the reaction mixture, is also provided herein.

### BRIEF DESCRIPTION OF THE DRAWINGS

FIG. 1A-1C Deletion of N. crassa CDH-1. (A) SDS-PAGE of proteins present in the culture filtrate of the wild type and the Δcdh-1 strain of N. crassa after 7 days of growth on AVICEL™. Missing protein band that corresponds to CDH-1 is marked by a box. (B) CDH activity in the culture filtrate of the wild-type and Δcdh-1 cultures as measured by the cellobiose-dependent reduction of DCPIP. Values are the mean of three biological replicates. Error bars are the SD between these replicates. (C) Avicelase activity of the wild-type and Δcdh-1 culture filtrates. Values are the mean of three biological replicates performed in technical triplicate. Error bars are the SD between these replicates.

FIG. 2A-2C Stimulation of cellulose (AVICEL™) degradation by the addition of M. thermophila CDH-1 to the Δcdh-1 culture filtrate. () Represents experiments where no exogenous CDH was added (∘) Represents experiments where 400 μg M. thermophila CDH-1 per gram of AVICEL™ was added. Avicelase assays with or without addition of M. thermophila CDH-1 to (A) Δcdh-1 N. crassa culture filtrate. (B) Wild-type N. crassa culture filtrate or (C) a mixture of purified cellulases (CBH-1, GH6-2, GH5-1, GH3-4) from N. crassa. Values are the mean of three replicates. Error bars are the SD between these replicates.

FIG. 3A-3D Stimulation of cellulose degradation by other isoforms of CDH. (A) Domain architectures of M. thermophila CDH-1 and CDH-2. Red c-terminal domain on CDH-1 is a fungal cellulose binding domain (CBM1). (B) AVICEL™ binding assay for M. thermophila CDH-1 and CDH-2. Lane 1 M. thermophila CDH-1, Lane 2 M. thermophila CDH-2, Lane 3 CDH-1 bound to AVICEL™, Lane 4 CDH-2 bound to AVICEL™. (C) Stimulation of cellulose degrading capacity of the Δcdh-1 culture filtrate () by addition of CDH-1 (∘), or CDH-2 (▾). (D) Effect of the concentration of M. thermophila CDH-1 and M. thermophila CDH-2 on Avicelase activity of the Δcdh-1 culture filtrates. Values are the mean of three replicates. Error bars are the SD between these replicates.

FIG. 4 Stimulation of cellulose degradation by domain truncations of CDH-2. Stimulation of cellulose degrading capacity of the Δcdh-1 culture filtrate () by addition of CDH-2 (▪), CDH-2 flavin domain (▾), or recombinant CDH-2 heme domain (♦). Values are the mean of three replicates. Error bars are the SD between these replicates.

FIG. 5A and FIG. 5B Metal and oxygen dependence of the stimulation of Avicelase activity by M. thermophila CDH1. (A) 10,000 fold buffer exchanged Δcdh-1 culture filtrate was treated with 100 uM EDTA and then reconstituted with various metal ions and Avicelase activity was analyzed after 45 hours of reaction. With the exception of the two leftmost columns, all samples were treated with EDTA and then reconstituted for 12 hours with 1.0 mM divalent metal ion. (B) Oxygen dependence of the stimulation of Avicelase activity by CDH. (Black) experiments conducted anaerobically, (Gray) experiments conducted aerobically. Values are the mean of three replicates. Error bars are the SD between these replicates.

FIG. 6A and FIG. 6B Stimulation of cellulose degradation by the addition of partially purified N. crassa CDH1 to the Δcdh-1 culture filtrate. (A) SDS-PAGE of partially purified N. crassa CDH1. (B) Avicelase activity of the Δcdh-1 culture filtrate. (∘) Represent experiments where 400 ug N. crassa CDH1 per gram of AVICEL™ was added. () Represent experiments where no exogenous CDH was added. Values are the mean of three replicates. Error bars are the SD between these replicates.

FIG. 7 SDS-PAGE of purified proteins used throughout the text. All proteins were loaded at 5 μg per lane in the following order: (1) M. thermophila CDH-1, (2) M. thermophila CDH-2, (3) M. thermophila CDH-2 flavin domain, (4) N. crassa CBH-1, (5) N. crassa GH6-2, (6) N. crassa GH5-1, (7) N. crassa GH3-4.

FIG. 8A and FIG. 8B Purity and spectral properties of recombinant CDH-2 heme domain expressed in Pichia pastoris. (A) SDS-PAGE of purified recombinant CDH-2 heme domain. (B) UV-vis spectra of the oxidized (black) and reduced (gray) CDH-2 heme domain.

FIG. 9 Avicelase activity of WT N. crassa culture broth () in the presence of 1.0 mM EDTA (∘). Values are the mean of three replicates. Error bars are the SD between these replicates.

FIG. 10 Metal dependence of the stimulation of Avicelase activity by M. thermophila CDH-1. (A) 10,000 fold buffer exchanged Δcdh-1 culture filtrate was treated with 100 uM EDTA and then reconstituted with various metal ions and Avicelase activity was analyzed after 45 hours of reaction. With the exception of the two leftmost columns, all samples were treated with EDTA and then reconstituted for 12 hours with 1.0 mM metal ion. Values are the mean of three replicates. Error bars are the SD between these replicates.

FIG. 11 Purification scheme of GH61 proteins. N. crassa Δcdh-1 was inoculated into Vogel's salts supplemented with 2% AVICEL™. After 7 days, cultures were filtered, concentrated, and separated over a MonoQ column then treated with 1.0 mM EDTA and repurified over a MonoQ column. Fractions containing cellulase enhancing activity dependent on the presence of CDH were finally purified over a gel filtration column.

FIG. 12 MonoQ fractionation of Δcdh-1 culture filtrate. Δcdh-1 culture filtrate was buffer exchanged into 25 mM Tris pH 8.5 and separated over a MonoQ anion exchange column using a gradient of NaCl. The load, flow-through, and all fractions were tested for the ability to stimulate cellulase activity in the presence of CDH by addition to a mixture of purified N. crassa cellulases and AVICEL™. In gel tryptic digests and LC-MS/MS were then performed to identify all proteins in active fractions; NCU01050, NCU02240, NCU07898, NCU08760 are indicated.

FIG. 13 Gel of purified N. crassa GH61 proteins. SDS-PAGE of native purified N. crassa GH61 proteins. Lane guide is as follows: L—Benchmark protein ladder, 1—NCU01050, 2—NCU02240, 3—NCU07898, 4—NCU08760.

FIG. 14 Cellulase assay of Zinc reconstituted N. crassa GH61 proteins. Following purification, the GH61 proteins were incubated at least 12 hours with 1 mM zinc sulfate. Pure GH61 proteins (0.02 mg/mL) were added to N. crassa cellulases (0.05 mg/mL CBH-1, GH6-2, and GH5-1; 0.005 mg/mL GH3-4) in the presence of M. thermophila CDH-1 (0.004 mg/mL) to look for the ability to stimulate cellulase activity. Unless otherwise noted all assays were performed with 10 mg/mL AVICEL™ in 50 mM sodium acetate pH 5.0 and 500 μM zinc sulfate at 40° C. The data is represented as the percent degradation at 24 hours relative to an assay lacking both CDH and GH61. All assays were performed in duplicate and error bars represent the range.

FIG. 15 Cellulase assay of EDTA treated N. crassa GH61 proteins. Pure, EDTA treated GH61 proteins (0.02 mg/mL) were added to N. crassa cellulases (0.05 mg/mL CBH-1, GH6-2, and GH5-1; 0.005 mg/mL GH3-4) in the presence of M. thermophila CDH-1 (0.004 mg/mL) to look for the ability to stimulate cellulase activity. All assays were performed with 10 mg/mL AVICEL™ in 50 mM sodium acetate pH 5.0 and 1.0 mM EDTA at 40° C. The data is represented as the percent degradation at 24 hours relative to an assay lacking both CDH and GH61. All assays were performed in duplicate and error bars represent the range.

FIG. 16 Pretreated corn stover assay of N. crassa GH61 proteins. Pure, zinc reconstituted GH61 proteins (NCU01050, NCU02240, NCU07898, NCU08760; 0.01 mg/mL each) were added to N. crassa cellulases (0.045 mg/mL CBH-1, GH6-2; 0.005 mg/mL GH3-4) in the presence (right bar) or absence (left bar) of M. thermophila CDH-1 (0.004 mg/mL) to look for the ability to stimulate cellulase activity. All assays were performed with 14 mg/mL washed NREL dilute acid pretreated corn stover in 50 mM sodium acetate pH 5.0 at 40° C. The data is represented as the percent degradation at 24 hours relative to an assay lacking both CDH and GH61. All assays were performed in triplicate and error bars represent the standard deviation.

FIG. 17 Multiple sequence alignment of GH61 proteins with sequence homology to NCU01050 and NCU02240. Multiple sequence alignments were performed locally using T-COFFEE (Notredame C, et al., J. Mol. Biol. 302, pp. 205-217 (2000)) and visualized using the Jalview multiple alignment editor (Waterhouse, A. M., et al. Bioinformatics 25, pp. 1189-1191 (2009)). Sequences in the alignment are provided as SEQ ID NOs: 52-69. All multiple sequence alignments of GH61 proteins were performed on curated GH61 sequences lacking the N-terminal signal peptide used to target the native protein for secretion.

FIG. 18 Maximum likelihood phylogeny of selected GH61 proteins showing sequence homology to NCU02240 and NCU01050. A maximum likelihood phylogeny of various proteins with homology to NCU02240 and NCU01050 was determined through a Phylogeny analysis (Dereeper A, et al. Nucleic Acids Res. 36, pp. W465-W469 (2008)). T-COFFEE was used for the multiple sequence alignment. There was no alignment curation and the tree was generated using the method of maximum likelihood with PhyML. Visualization of the tree was done using TreeDyn. Sequences in the alignment are provided as SEQ ID NOs: 52-59.

FIG. 19 Identification of native metal ligation in GH61 proteins. Neurospora crassa containing a deletion of cdh-1 was grown on Vogel's salts media supplemented with 2% w/v AVICEL™ PH101 and 5 uM copper(II) sulfate for 7 days at 25 C and 200 RPM shaking. Fungus was removed from culture by filtration over 0.2 micron PES filters. The culture filtrate was concentrated using tangential flow filtration and buffer exchanged into 25 mM TRIS pH 8.5. The concentrated and buffer exchanged filtrate was loaded onto a 10/100 GL MonoQ column and fractionated into 5 fractions with a linear salt gradient. Each fraction was then analyzed for the presence of copper or zinc. Metal analysis was performed using a Perkin Elmer inductively coupled plasma atomic emission spectrometer. The bar graph shows the amount of zinc and copper in each of the fractions from the MonoQ column. For each set of 2 bars, the copper is on the left, and the zinc is on the right. The image is of an SDS-PAGE of each of the fractions. The boxes on the gel are around the known GH61 proteins. The results of these experiment show that the highest amounts of copper are found in the fractions that contain GH61 proteins (the flow-through (FT) and Fraction A2).

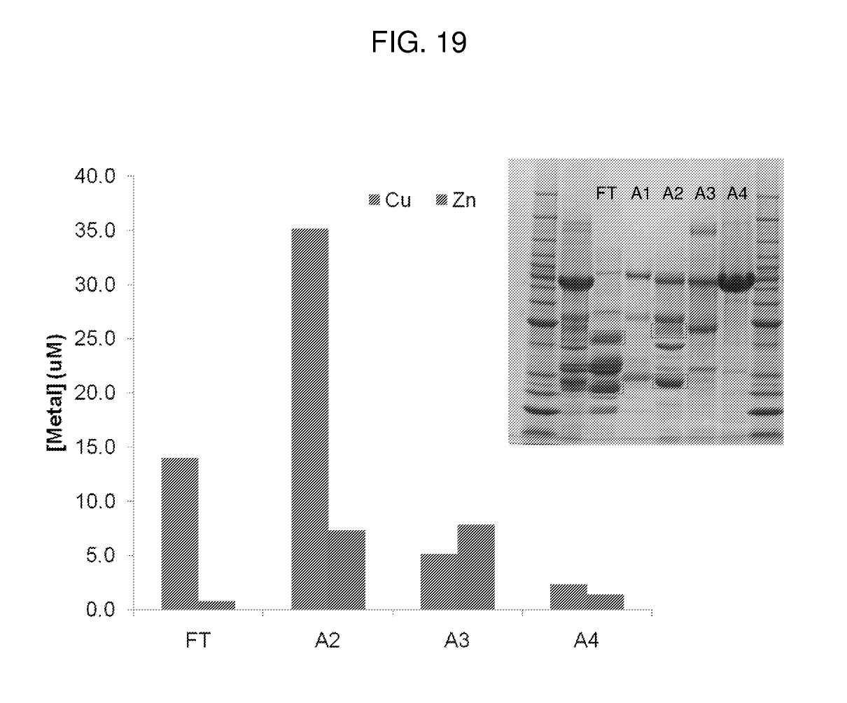

FIG. 20 Metal stoichiometry of purified NCU01050. Apo NCU01050 stock in 25 mM TRIS pH 8.5 and 150 mM sodium chloride was diluted to ˜1 mg/mL in a total volume of 1 mL. Copper sulfate, zinc sulfate, or a 1:1 mixture of copper and zinc sulfate were added to the protein to a final concentration of 100 uM of each metal and the samples left overnight at room temperature (12-16 hours). Samples were then buffer exchanged into 25 mM TRIS pH 8.5 using a 26/10 desalting column. The desalted protein was concentrated to a final volume of 2-2.5 mL using 3000 MWCO polyethersulfone spin concentrators. The absorbance at 280 nm was then recorded and used to calculate total protein concentration. The flow through from the spin concentrator was also saved as a blank. Metal analysis was performed using a Perkin Elmer inductively coupled plasma atomic emission spectrometer. The bar graph shows the amount of zinc and copper in the NCU01050 which was incubated with copper, zinc, or a mixture of copper and zinc. For each set of 2 bars, the copper is on the left, and the zinc is on the right. The results of this experiment support that both copper and zinc can bind to NCU01050, however in the presence of equimolar quantities of both metals, copper is the preferred metal.

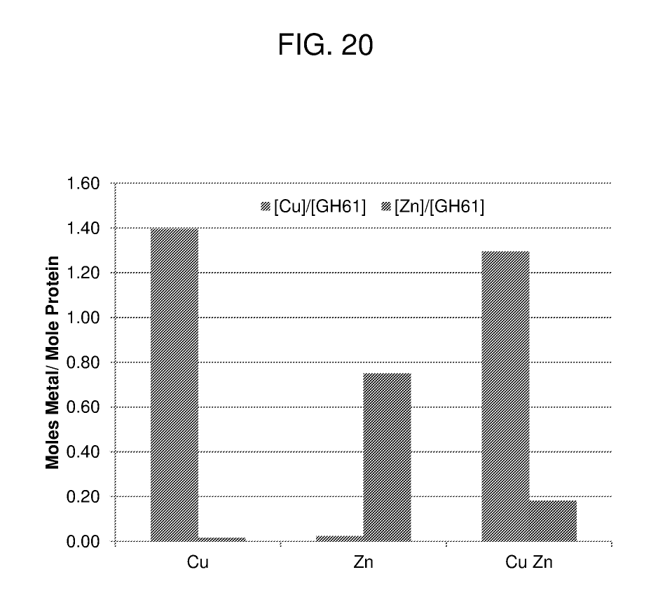

FIG. 21 Metal stoichiometry of purified NCU07898. Apo NCU07898 stock in 25 mM TRIS pH 8.5 and 150 mM sodium chloride was diluted to ˜1 mg/mL in a total volume of 1 mL. Copper sulfate, zinc sulfate, or a 1:1 mixture of copper and zinc sulfate were added to the protein to a final concentration of 100 uM of each metal and the samples left overnight at room temperature (12-16 hours). Samples were then buffer exchanged into 25 mM TRIS pH 8.5 using a 26/10 desalting column. The desalted protein was concentrated to a final volume of 2-2.5 mL using 3000 MWCO polyethersulfone spin concentrators. The absorbance at 280 nm was then recorded and used to calculate total protein concentration. The flow through from the spin concentrator was also saved as a blank. Metal analysis was performed using a Perkin Elmer inductively coupled plasma atomic emission spectrometer. The bar graph shows the amount of zinc and copper in the NCU07898 which was incubated with copper, zinc, or a mixture of copper and zinc. For each set of 2 bars, the copper is on the left, and the zinc is on the right. The results of this experiment support that both copper and zinc can bind to NCU07898, however in the presence of equimolar quantities of both metals, copper is the preferred metal.

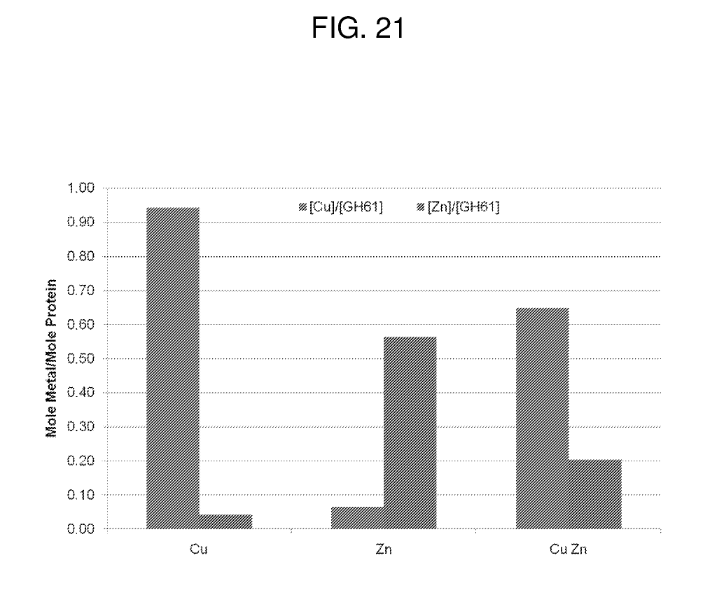

FIG. 22 Metal stoichiometry of purified NCU08760. Apo NCU08760 stock in 25 mM TRIS pH 8.5 and 150 mM sodium chloride was diluted to ˜1 mg/mL in a total volume of 1 mL. Copper sulfate, zinc sulfate, or a 1:1 mixture of copper and zinc sulfate were added to the protein to a final concentration of 100 uM of each metal and the samples left overnight at room temperature (12-16 hours). Samples were then buffer exchanged into 25 mM TRIS pH 8.5 using a 26/10 desalting column. The desalted protein was concentrated to a final volume of 2-2.5 mL using 3000 MWCO polyethersulfone spin concentrators. The absorbance at 280 nm was then recorded and used to calculate total protein concentration. The flow through from the spin concentrator was also saved as a blank. Metal analysis was performed using a Perkin Elmer inductively coupled plasma atomic emission spectrometer. The bar graph shows the amount of zinc and copper in the NCU08760 which was incubated with copper, zinc, or a mixture of copper and zinc. For each set of 2 bars, the copper is on the left, and the zinc is on the right. The results of this experiment support that both copper and zinc can bind to NCU08760.

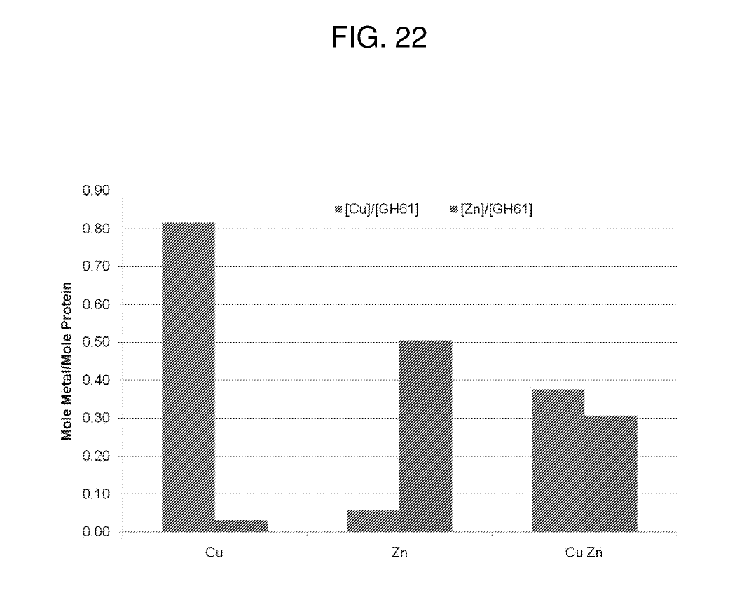

FIG. 23 Activity of M. thermophila CDH-2 is enhanced by NCU01050. In this experiment 0.01 mg/mL of MT CDH-2 was incubated with 1.0 mM cellobiose for 30 minutes and the product of the reaction, cellobionic acid, was analyzed using HPLC (dionex). If the CDH is incubated with 10 uM copper and the cellobiose, only 0.24 (in arbitrary units) cellobionic acid is produced. If NCU01050 is added, the amount of cellobionic acid produced is increased by ˜36 fold to 8.74 units. If 1.0 mM of EDTA is added to the CDH/NCU01050/Copper mix, only 0.56 units are formed. This data indicates that the presence of NCU01050 enhances the rate of oxidation of cellobiose by CDH-2.

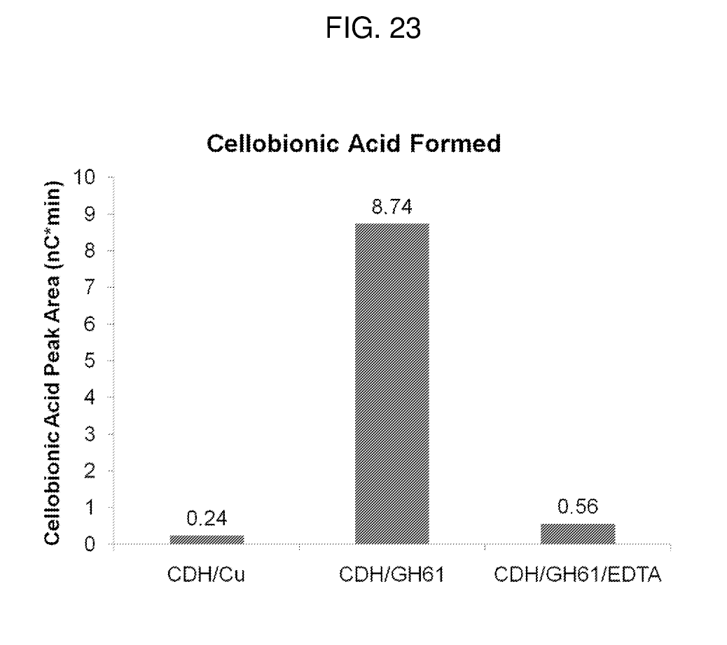

FIG. 24 Copper dependence of oxidized product. NCU01050/GH61-4 was purified natively from N. crassa and extensively treated with EDTA to remove all metals. The protein was determined to be >95% apo (metal-free) by ICP-AES and was then reconstituted for one hour with a 10-fold molar excess of Zinc or Cuprous sulfate. To determine the metal dependence of the GH61 reaction, an assay was performed on 5 mg/mL AVICEL™. All assays were performed in 10 mM Na Acetate pH 5.0 at 40° C. and contained N. crassa CBH-1 (0.035 mg/mL) and CBH-2 (0.015 mg/mL). Then, CDH (0.005 mg/mL), NCU01050/GH61-4 (concentration listed on graph), or a combination of the two were added to the cellulases. After 30 hours of incubation reactions were centrifuged, the assay supernatant was diluted 5-fold and loaded onto a dionex HPAEC. For dionex analysis the CarboPac PA200 HPAEC column was used in 0.1M NaOH and a gradient was ran from 0-160 mM Na Acetate over 16 minutes followed by a 5 minute flush in 300 mM Na Acetate and a 3 minute equilibration in 0 mM Na Acetate. A distinct set of peaks eluted at 20-23 minutes and these peaks are only present in samples containing both CDH and GH61. The retention time is significantly later than any cello-oligosaccharide generated by cellulases or their acid products that result from CDH oxidation at the C1 carbon. This new product on the Dionex was significantly larger with Copper bound enzyme relative to Zinc bound enzyme. The area of the new peak generated by 1 uM zinc bound GH61 in the presence of CDH was roughly the same size as a similar reaction containing 40-fold less copper bound GH61. The bar graph shows the relative size of the peak area of the new product on the Dionex. For each set of 2 bars, the amount of product from the reaction with the GH61 protein that was reconstituted with zinc is on the left, and the amount of product from the reaction with the GH61 protein that was reconstituted with copper is on the right. All reagents used in this assay were Sigma Traceselect grade and the enzymes and AVICEL™ were extensively EDTA treated and washed to remove all metal contaminants from the assay.

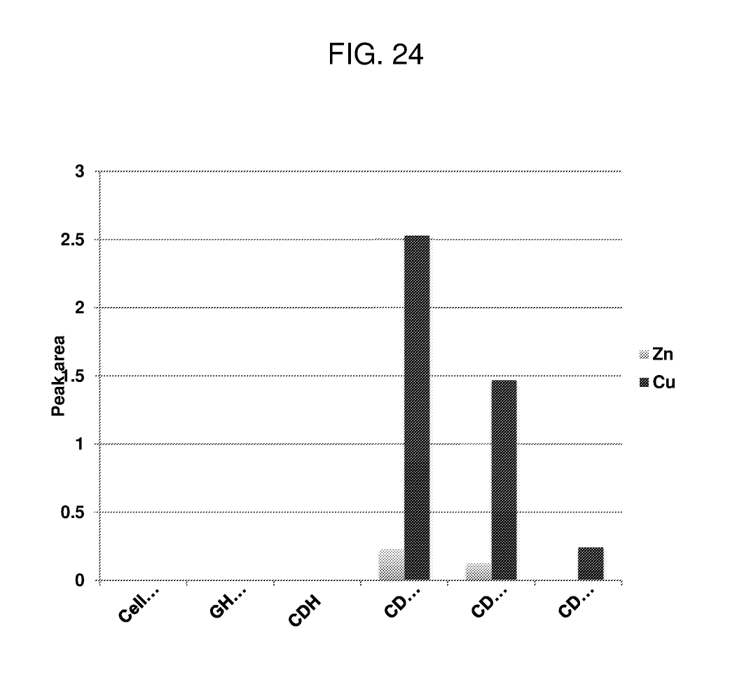

FIG. 25 The His, Gln, and Tyr residues of the motif H-X₍₄₋₈₎-Q-X-Y of GH61 polypeptides are important for GH61 polypeptide activity. N. crassa NCU08760 polypeptides having H179A (“HA”), Q188A (“QA”), or Y190F (“YF”) mutations were prepared. These different mutant NCU08760 polypeptides, as well as wild-type (“WT”) NCU08760 were assayed for activity on phosphoric acid swollen cellulose (“PASC”). The X-axis indicates the enzyme and concentration (in μm), and the Y-axis indicates Pk Area (acids).

### DETAILED DESCRIPTION OF EMBODIMENTS

The present disclosure relates to compositions and methods for degrading cellulose. These compositions and methods provide a dramatic improvement in cellulose degradation over prior polypeptides, polynucleotides, compositions and methods. In some embodiments, the present disclosure relates to novel polypeptides, and polynucleotides encoding the polypeptides. In some embodiments, the present disclosure relates to methods for identifying CDH-dependent accessory cellulase systems.

Disclosed herein are compositions and methods involving cellobiose dehydrogenase (CDH)-heme domain polypeptides. The protein CDH was originally identified in Phanerochaete chrysosporium (“P. chrysosporium”), and CDH orthologs have been identified in multiple species of fungi, including Neurospora crassa (“N. crassa”).

CDH proteins contain an N-terminal heme domain and a C-terminal dehydrogenase domain. Some CDH proteins also contain a cellulose binding module (CBM) at the C-terminus of the protein. Orthologs of the CDH heme domain are found only in fungal proteins, whereas orthologs of the dehydrogenase domain are found in proteins throughout all domains of life; the dehydrogenase domain is part of the larger GMC oxidoreductase superfamily. Crystal structures of heme and flavin domain from P. chrysosporium have been determined. (Zamocky et al., Curr. Prot. Pept. Sci., Vol. 7, No. 3, pp. 255-280, (2006)).

A non-naturally occurring polypeptide having a first domain containing a CDH-heme domain and a second domain containing a cellulose binding module (CBM) is provided herein. These polypeptides are more effective at increasing degradation of cellulose than otherwise equivalent CDH-heme domain containing-polypeptides which lack a CBM. It is also possible to increase the degradation of cellulose with fewer of these polypeptides than with otherwise equivalent CDH-heme domain containing-polypeptides which lack a CBM.

A non-naturally occurring polypeptide having a first domain containing a CDH-heme domain and a second domain containing a cellulose binding module (CBM), and not containing a dehydrogenase domain is also provided herein. These polypeptides may cause less oxidative damage to molecules in a cellulase reaction and reduce the formation of reactive oxygen species in a cellulase reaction, as compared to otherwise equivalent polypeptides that have a CDH-heme domain and a CBM, but which also have a dehydrogenase domain. Oxidative damage to molecules in a cellulase reaction may result in, for example, one or more of: impairment of enzyme activity, chemical alteration of enzyme substrates or products, or the generation of undesirable side products.

CDH-heme polypeptides disclosed herein have higher activity under aerobic conditions than under anaerobic conditions.

As used herein, “CDH protein” refers to a polypeptide having the amino acid sequence of N. Crassa CDH-1 (SEQ ID NO: 32), N. Crassa CDH-2 (SEQ ID NO: 43), M. thermophila CDH-1 (SEQ ID NO: 46), M. thermophila CDH-2 (SEQ ID NO: 49), or other polypeptide occurring in nature having a CDH-heme domain (discussed below) and a dehydrogenase domain. CDH proteins in different organisms may be identified through sequence identity/homology to known CDH proteins, and examples of CDH proteins include, without limitation, the polypeptides of Accession Numbers: XM_411367, BAD32781, BAC20641, XM_389621, AF257654, AB187223, XM_360402, U46081, AF081574, AY187232, AF074951, and AF029668. “CDH protein” also refers to conservatively modified variants of naturally occurring CDH proteins. “CDH protein” also includes CDH proteins with and without an intact signal peptide. CDH proteins may be secreted by cells, and have a short (around 15-25 amino acid) signal sequence at the N-terminus of the cDNA translation product, which targets the protein for secretion and is cleaved in the mature CDH protein.

Also disclosed herein are compositions and methods involving glycoside hydrolase family 61 polypeptides (“GH61” polypeptides). GH61 polypeptides are a large group of polypeptides having a sequence classified as provided in the NCBI conserved domains identifier: c104076, the NCBI name: glycol_hydro 61, and the Pfam protein family number: pfam03443.

GH61 polypeptides disclosed herein may be provided with mixtures that contain cellulases and cellulose-containing material to increase the degradation of cellulose-containing material in these mixtures, as compared to degradation of cellulose-containing material in otherwise equivalent mixtures to which the GH61 polypeptides are not added.

Recombinant GH61 polypeptides that are bound to a copper atom are also provided. These GH61 polypeptides may be more effective at increasing degradation of cellulose than otherwise equivalent GH61 polypeptides which are not bound to a copper atom.

Also provided are compositions containing a recombinant GH61 polypeptide and a recombinant CDH-heme domain polypeptide containing a CBM. These compositions may include various GH61 polypeptides and CDH-heme domain polypeptides disclosed herein. These compositions may be included with mixtures that contain cellulases and cellulose-containing material to increase degradation of cellulose-containing material, as compared to degradation of cellulose-containing material in otherwise equivalent mixtures to which these compositions are not added.

### Variants, Sequence Identity, and Sequence Similarity

Methods of alignment of sequences for comparison are well-known in the art. For example, the determination of percent sequence identity between any two sequences can be accomplished using a mathematical algorithm. Non-limiting examples of such mathematical algorithms are the algorithm of Myers and Miller (1988) CABIOS 4:11 17; the local homology algorithm of Smith et al. (1981) Adv. Appl. Math. 2:482; the homology alignment algorithm of Needleman and Wunsch (1970) J. Mol. Biol. 48:443 453; the search-for-similarity-method of Pearson and Lipman (1988) Proc. Natl. Acad. Sci. 85:2444 2448; the algorithm of Karlin and Altschul (1990) Proc. Natl. Acad. Sci. USA 872264, modified as in Karlin and Altschul (1993) Proc. Natl. Acad. Sci. USA 90:5873 5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237 244 (1988); Higgins et al. (1989) CABIOS 5:151 153; Corpet et al. (1988) Nucleic Acids Res. 16:10881 90; Huang et al. (1992) CABIOS 8:155 65; and Pearson et al. (1994) Meth. Mol. Biol. 24:307 331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTX for proteins) can be used. Alignment may also be performed manually by inspection.

As used herein, sequence identity or identity in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical and often differ by conservative amino acid substitutions, where amino acid residues are substituted for other amino acid residues with similar chemical properties (e.g., charge or hydrophobicity), do not change the functional properties of the molecule. When sequences differ in conservative substitutions, the percent sequence identity may be adjusted upwards to correct for the conservative nature of the substitution. Sequences that differ by such conservative substitutions are said to have sequence similarity or similarity. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

The functional activity of enzyme variants can be evaluated using standard molecular biology techniques including thin layer chromatography and high performance liquid chromatography to assay enzymatic products. Enzymatic activity can be determined using substrates including cellobiose, crystalline cellulose, such as AVICEL™, and lignocellulosic materials.

### CDH-Heme Domain

Polypeptides containing a CDH-heme domain are provided herein. As used herein, “CDH-heme domain” refers to a polypeptide having an amino acid sequence that is identical to or homologous to an amino acid sequence of the heme domain of a CDH protein. CDH-heme domains are well characterized and known to one of skill in the art. The crystal structure of the CDH-heme domain from Phanerochaete chrysosporium CDH protein has been determined (Hallberg, B. M. et al. Structure (9), pp. 79-88 (2000); and (Zamocky, M. et al., Curr. Prot. Pept. Sci., (7), 3, pp. 255-280, (2006))), and the sequence of many CDH-heme domains have been identified. Examples of CDH-heme domain amino acid sequences include SEQ ID NOs: 1-23, 70 (N. crassa CDH-1 heme), 76 (N. crassa CDH-2 heme), 80 (M. thermophila CDH-1 heme), and 86 (M. thermophila CDH-2 heme).

CDH-heme domains are approximately 175-225 amino acids in length, and have a heme prosthetic group that is coordinated through a methionine and a histidine residue. In addition, CDH-heme domains have conserved spectral properties, due to the conserved methionine/histidine coordination of the heme group. CDH-heme domains may be identified by various techniques, including amino acid or nucleic acid sequence homology to known CDH-heme domains, spectral properties as compared to known CDH-heme domains, and three-dimensional structure as compared to known CDH-heme domains. As would be understood by one of skill in the art, polypeptides having low amino acid sequence similarity may still have highly similar spectral properties and/or three-dimensional structures.

As provided herein, “CDH-heme domains” include polypeptides having the amino acid sequences provided in SEQ ID NOs: 1-23, 70 (N. crassa CDH-1 heme), 76 (N. crassa CDH-2 heme), 80 (M. thermophila CDH-1 heme), 86 (M. thermophila CDH-2 heme). “CDH-heme domains” also includes polypeptides having at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, sequence identity/sequence similarity to any of the polypeptides of SEQ ID NOs: 1-23, 70, 76, 80, 86. “CDH-heme domains” also includes polypeptides having a heme group coordinated through a methionine and a histidine residue, and having spectral properties and/or three dimensional characteristics that identify the polypeptide to one of skill in the art as being homologous or orthologous to any of the polypeptides of SEQ ID NOs: 1-23, 70, 76, 80, 86.

### Cellulose Binding Module (CBM)

Polypeptides containing a cellulose binding module (CBM) are also provided herein. A CBM is an amino acid sequence which adopts a three-dimensional conformation that has carbohydrate binding activity, and which may be part of a larger protein having carbohydrate-related enzymatic activity. As used herein “CBM” refers any polypeptide having a discrete fold with carbohydrate binding activity. In one aspect, a CBM of the present disclosure may bind cellulose.

CBMs have been organized into various CBM “families” based on amino acid sequence, protein fold structure, and/or binding specificity. Information about CBMs is provided, for example, in Boraston A. et al., Biochem. J. 382, pp. 769-781 (2004) and Shoseyov O. et al., Micro. Mol. Biol. Rev. (70) 2, pp. 283-295 (2006).

CBMs of the present disclosure include “CBM Family 1” CBMs. CBM Family 1 CBMs are around 40 amino acids in length, and naturally occur almost exclusively in fungi. CBM Family 1 CBMs have well-characterized cellulose-binding properties. CBM Family 1 CBMs have the National Center for Biotechnology Information (NCBI) conserved domain identifier: c102521, and the NCBI name: CBM_1. CBM Family 1 CMBs also have the InterPro protein database accession number: IPR000254, and the Pfam protein database family number: pf00734.

CBMs of the present disclosure also include “CBM Family 2” CBMs. CBM Family 2 CBMs are around 100 amino acids in length, and naturally occur primarily in bacteria. CBM Family 2 CBMs have well-characterized cellulose-binding properties. CBM Family 2 CMBs have the NCBI conserved domain identifier: c102709, and the NCBI name: CBM_2. CBM Family 2 CMBs also have the InterPro protein database accession number: IPR001919, and the Pfam protein database family number: pf00553.

CBMs of the present disclosure also include “CBM Family 3” CBMs. CBM Family 3 CBMs are around 150 amino acids in length, and naturally occur in bacteria. CBM Family 3 CBMs have well-characterized cellulose-binding properties. CBM Family 3 CMBs have the NCBI conserved domain identifier: c103026, and the NCBI name: CBM_3. CBM Family 3 CMBs also have the InterPro protein database accession number: IPR001956, and the Pfam protein database family number: pfam00942.

CBMs of the present disclosure also include “CBM Family 8” CBMs. CBM Family 8 CBMs have been identified in the slime mold Dictyostelium discoideum. For example, the polypeptide of GenBank accession number AAA52077.1 contains a CBM Family 8 CMB.

CBMs of the present disclosure also include “CBM Family 9” CBMs. CBM Family 9 CBMs are around 170 amino acids in length, and have been identified in xylanases. CBM Family 9 CMBs include the NCBI conserved domain identifiers: cd00005, cd09620, and cd09619 and the NCBI names: CBM9_like_1, CBM9_like_3, and CBM9_like_4. CBM Family 9 CMBs also include the InterPro protein database accession number: IPR003305, and the Pfam protein family number: pf02018.

CBMs of the present disclosure also include “CBM Family 10” CBMs. CBM Family 10 CBMs are around 50 amino acids in length. CBM Family 10 CMBs have the NCBI conserved domain identifier: c107836, and the NCBI name: CBM_10. CBM Family 10 CMBs also have the InterPro protein database accession number: IPR002883, and the Pfam protein family number: pfam02013.

CBMs of the present disclosure also include “CBM Family 11” CBMs. CBM Family 11 CBMs are around 180-200 amino acids in length. CBM Family 9 CMBs have NCBI conserved domain identifier: c104062, and the NCBI name: CMB_11. CBM Family 9 CMBs also have the Pfam protein family number: pfam03425.

CBMs of the present disclosure also include “CBM Family 16”, “CBM Family 30”, “CBM Family 37”, “CBM Family 44”, “CBM Family 46”, “CBM Family 49”, “CBM Family 59”, and “CBM Family 28” CBMs.

CBMs of the present disclosure also include “CBM Family 4” CBMs. CBM Family 4 CBMs are around 150 amino acids in length, and naturally occur in bacteria. CBM Family 4 CMBs have the NCBI conserved domain identifier: c103406, and the NCBI name: CBM_4_9. CBM Family 4 CMBs also have the InterPro protein database accession number: IPR003305, and the Pfam protein family number: pfam02018.

CBMs of the present disclosure also include “CBM Family 6” CBMs. CBM Family 6 CBMs are around 120 amino acids in length. CBM Family 6 CMBs have the NCBI conserved domain identifier: c102697, and the NCBI name: CBM_6. CBM Family 6 CMBs also have the InterPro protein database accession number: IPR005084, and the Pfam protein family number: pfam03422.

CBMs of the present disclosure also include “CBM_17 Family” CBMs. CBM Family 17 CBMs are around 200 amino acids in length. CBM Family 17 CMBs have the NCBI conserved domain identifier: c104061, and the NCBI name: CBM_17_28. CBM Family 17 CMBs also have the InterPro protein database accession number: IPR005086, and the Pfam protein family number: pfam03424.

CBMs of the present disclosure also include polypeptides having the amino acid sequence of the CBM of N. crassa CDH-1 or the CBM of M. thermophila CDH-1. The amino acid sequence of the CBM of N. crassa CDH-1 is provided in SEQ ID NO: 74 and the CBM of M. thermophila CDH-1 is provided in SEQ ID NO: 84.

CBM domains of the present disclosure include recombinant polypeptides having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity/sequence similarity to the polypeptide of SEQ ID NO: 74 (CBM of N. crassa CDH-1) or SEQ ID NO: 84 (CBM of M. thermophila CDH-1).

### Dehydrogenase Domain

Polypeptides containing a dehydrogenase domain are also provided herein. Dehydrogenase domains are also referred to herein as “oxidative domains.” Polypeptides having a dehydrogenase domain are also herein referred to as “dehydrogenases.” Dehydrogenases may oxidize a substrate (e.g. cause the substrate to lose electrons/have an increase in oxidation number) and reduce an acceptor (e.g. cause the acceptor to gain electrons/have a decrease in oxidation number).

A dehydrogenase domain of the present disclosure is a dehydrogenase domain of the GMC oxidoreductase superfamily. Dehydrogenase domains of the present disclosure also include dehydrogenase domains of the GMC oxidoreductase N superfamily. GMC oxidoreductase N superfamily dehydrogenase domains have the NCBI conserved domain identifier: c102950, and the NCBI name: GMC_oxred_N. GMC oxidoreductase N superfamily dehydrogenase domains have the Pfam protein family number: pf00732. Dehydrogenase domains of the present disclosure also include dehydrogenase domains of the GMC oxidoreductase C superfamily. GMC oxidoreductase C superfamily dehydrogenase domains have the NCBI conserved domain identifier: c108434, and the NCBI name: GMC_oxred_C. GMC oxidoreductase N superfamily dehydrogenase domains also have the Pfam family number: pf00732.

Dehydrogenase domains of the present disclosure include the dehydrogenase domains of N. crassa CDH-1, N. crassa CDH-2, M. thermophila CDH-1, and M. thermophila CDH-2. In both N. crassa and M. thermophila CDH dehydrogenase domains, a flavin group is present. As used herein, the dehydrogenase domain of N. crassa CDH-1, M. thermophila CDH-1, and homologous CDH proteins is also referred to as a “flavin” domain.

Another dehydrogenase domain of the present disclosure is the glucose/sorbosone dehydrogenase domain of the Coprinopsis cinera (“C. cinera”) polypeptide XP_001837973.1 (SEQ ID NO: 50), which has a CDH-like heme domain, a glucose/sorbosone dehydrogenase domain, and a fungal cellulose binding domain. The sequence of the dehydrogenase domain of XP_001837973.1 is provided in SEQ ID NO: 51.

Dehydrogenase domains of the present disclosure include recombinant polypeptides having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity/sequence similarity to the polypeptide of: SEQ ID NO: 72 (dehydrogenase domain of N. crassa CDH-1); SEQ ID NO: 78 (dehydrogenase domain of N. crassa CDH-2); SEQ ID NO: 82 (dehydrogenase domain of M. thermophila CDH-1); SEQ ID NO: 88 (dehydrogenase domain of M. thermophila CDH-2), or SEQ ID NO: 51 (dehydrogenase domain of C. cinera XP_001837973.1).

### Polypeptides of the Disclosure

As used herein, a “polypeptide” is an amino acid sequence including a plurality of consecutive polymerized amino acid residues (e.g., at least about 15 consecutive polymerized amino acid residues). A polypeptide optionally contains modified amino acid residues, naturally occurring amino acid residues not encoded by a codon, and non-naturally occurring amino acid residues.

As used herein, “protein” refers to an amino acid sequence, oligopeptide, peptide, polypeptide, or portions thereof whether naturally occurring or synthetic.

As used herein, a “non naturally-occurring” polypeptide refers to a polypeptide sequence that has an overall amino acid sequence that is not found in nature (i.e. even if a polypeptide contains one or more subsequences that are found in nature, if the overall amino acid sequence of the polypeptide is not found it nature, it is considered a “non naturally-occurring” polypeptide as used herein).

As used herein, a “recombinant” polypeptide refers to a polypeptide sequence wherein at least one of the following is true: (a) the sequence of the polypeptide is foreign to (i.e., not naturally found in) a given host cell; (b) the sequence of the polypeptide may be naturally found in a given host cell, but in an unnatural (e.g., greater than expected) amount; or (c) the overall sequence of the polypeptide does not exist in nature.

As used herein, a polypeptide sequence that is “derived from” a naturally occurring sequence may be identical to the naturally occurring sequence, or it may have differences from the naturally occurring sequence.

### CDH-Heme Domain Polypeptides

CDH-heme domain polypeptides are provided herein. As used herein, a “CDH-heme domain polypeptide” includes any polypeptide having a CDH-heme domain.

CDH-heme domain polypeptides include recombinant CDH proteins. CDH-heme domain polypeptides also include non-naturally occurring CDH-heme domain polypeptides (discussed below). CDH-heme domain polypeptides may lack a CBM and a dehydrogenase domain.

### Non-Naturally Occurring CDH-Heme Domain Polypeptides

Non-naturally occurring CDH-heme domain polypeptides are provided herein. A non-naturally occurring CDH-heme domain polypeptide is any polypeptide that contains a CDH-heme domain and that has an overall amino acid sequence that is not found in nature.

A non-naturally occurring CDH-heme domain polypeptide may contain two or more polypeptide subsequences and/or domains that occur in nature, but that are situated in the non-naturally occurring CDH-heme polypeptide chain in a different relationship to each other than occurs in nature. In one format, the subsequences and/or domains in the non-naturally occurring are separated by fewer amino acids in the non-naturally occurring CDH-heme polypeptide chain than occurs in a naturally occurring polypeptide. In another format, the subsequences and/or domains in the non-naturally occurring are separated by more amino acids in the non-naturally occurring CDH-heme polypeptide chain than occurs in a naturally occurring polypeptide. In another format, the subsequences and/or domains in the non-naturally occurring polypeptide are in a different order in the non-naturally occurring CDH-heme polypeptide chain than occurs in a naturally occurring polypeptide. In another format, the subsequences and/or domains in the non-naturally occurring polypeptide are in a different order in the non-naturally occurring CDH-heme polypeptide chain than occurs in a naturally occurring polypeptide. In another format, the subsequences and/or domains in the non-naturally occurring polypeptide do not occur together in a naturally occurring polypeptide

### Non-Naturally Occurring Polypeptides Containing a CDH-Heme Domain and CBM

A non-naturally occurring CDH-heme domain polypeptide having a CDH-heme domain and a CBM is provided herein. A CDH-heme domain polypeptide having a CDH-heme domain and a CBM may optionally include a dehydrogenase domain.

In a non-naturally occurring polypeptide having a CDH-heme domain and a CBM, the CDH-heme domain may be directly linked with the CBM in the polypeptide chain. In other format, the CDH-heme domain and the CBM may be separated in the polypeptide chain by one or more amino acids. In some aspects, the CDH-heme domain and the CBM may be separated by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 150, 200, 250, 300, 350, 400, 450, 500, 550, 600, 650, 700, 750, 800, 850, 900, 950, or 1000 amino acids in the polypeptide chain.

The CDH-heme domain and the CBM may be arranged in any order in the polypeptide chain of a non-naturally occurring polypeptide having a CDH-heme domain and a CBM. For example, the CDH-heme domain may be N-terminal to the CBM on the polypeptide chain, or C-terminal to the CBM on the polypeptide chain.

The CDH-heme domain and the CBM of a non-naturally occurring polypeptide having a CDH-heme domain and a CBM may be derived from the same species of CDH protein (e.g. from the same CDH gene). For example, the CDH-heme domain and the CBM may be derived from N. crassa CDH-1 (SEQ ID NO: 32), so that the CDH-heme domain has the sequence of SEQ ID NO: 70 and the CBM has the sequence of SEQ ID NO: 74. As another example, the CDH-heme domain and the CBM may be derived from M. thermophila CDH-1 (SEQ ID NO: 46), so that the CDH-heme domain has the sequence of SEQ ID NO: 80 and the CBM has the sequence of SEQ ID NO: 84.

In another format, the CDH-heme domain and the CBM of a non-naturally occurring polypeptide having a CDH-heme domain and a CBM are not derived from the same species of CDH protein. For example, the CDH-heme domain may be derived from a CDH protein, and the CBM may be derived from a non-CDH protein. In another example, the CDH-heme domain is derived from one species of CDH protein, and the CBM is derived from a different species CDH protein (e.g. CDHs of two different CDH genes).

A non-naturally occurring polypeptide having a CDH-heme domain and a CBM may be more effective at increasing degradation of cellulose than an equivalent or similar polypeptide that lacks a CBM. A non-naturally occurring polypeptide having a CDH-heme domain and a CBM may be at least about 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, or 1000% more effective at increasing degradation of cellulose than an equivalent or similar polypeptide that lacks a CBM.

Examples of a first polypeptide being “more effective at increasing degradation of cellulose” than a second polypeptide include, without limitation: i) if an equivalent number of molecules of a first and second polypeptide are provided to two separate cellulase-containing reactions containing the same reaction conditions (so that the first polypeptide is added to one reaction, and the second polypeptide is added to the other reaction), and the first polypeptide increases the rate of degradation of cellulose in its reaction more than the second polypeptide increases the rate of degradation of cellulose in its reaction; ii) if an equivalent number of molecules of a first and second polypeptide are provided to two separate cellulase-containing reactions containing the same reaction conditions (so that the first polypeptide is added to one reaction, and the second polypeptide is added to the other reaction), and the first polypeptide increases the extent of degradation of cellulose in its reaction more than the second polypeptide increases the extent of degradation of cellulose in its reaction; iii) if fewer molecules of a first polypeptide than a second polypeptide are required to increase the rate of degradation of cellulose in a cellulase-containing reaction to a target rate of cellulose degradation.

A non-naturally occurring polypeptide having a CDH-heme domain and a CBM that increases degradation of cellulose more than an equivalent or similar polypeptide that lacks a CBM is also provided. For example, a non-naturally occurring polypeptide having a CDH-heme domain and a CBM may increase degradation of cellulose by about 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, or 1000% more than an equivalent or similar polypeptide that lacks a CBM, under the same reaction conditions.

A non-naturally occurring polypeptide having a CDH-heme domain and a CBM but lacking a dehydrogenase domain may result in less oxidative damage to molecules in a cellulase reaction than an otherwise equivalent polypeptide having a dehydrogenase domain.

### Non-Naturally Occurring Polypeptides Containing a CDH-Heme Domain, a CBM, and a Dehydrogenase Domain

A non-naturally occurring polypeptide having a CDH-heme domain, a CBM, and a dehydrogenase domain is also provided.

In these polypeptides, the CDH-heme domain, the CBM, and the dehydrogenase domain may be directly linked in the polypeptide chain. Alternatively, one or more of the CDH-heme domain, the CBM, and the dehydrogenase domain may be separated in the polypeptide chain by one or more amino acids. For example, the CDH-heme domain, the CBM, and the dehydrogenase domain may be separated from each other by any of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 150, 200, 250, 300, 350, 400, 450, 500, 550, 600, 650, 700, 750, 800, 850, 900, 950, or 1000 amino acids in the polypeptide chain.

In a non-naturally occurring polypeptide having a CDH-heme domain, a CBM, and a dehydrogenase domain, the CDH-heme domain, the CBM, and the dehydrogenase domain may be arranged in any order in the polypeptide chain. For example, the CDH-heme domain may be N-terminal to both the CBM and the dehydrogenase domain in the polypeptide chain, or it may be C-terminal to both the CBM and the dehydrogenase domain in the polypeptide chain, or it may be between the CBM and the dehydrogenase domain in the polypeptide chain. Similarly, the CBM may be N-terminal to both the CDH-heme domain and the dehydrogenase domain in the polypeptide chain, or it may be C-terminal to both the CDH-heme domain and the dehydrogenase domain in the polypeptide chain, or it may be between the CDH-heme domain and the dehydrogenase domain in the polypeptide chain. Similarly, the dehydrogenase domain may be N-terminal to both the CDH-heme domain and the CBM in the polypeptide chain, or it may be C-terminal to both the CDH-heme domain and the CBM in the polypeptide chain, or it may be between the CDH-heme domain and the CBM in the polypeptide chain.

In a non-naturally occurring polypeptide having a CDH-heme domain, a CBM, and a dehydrogenase domain, the CDH-heme domain, the CBM, and the dehydrogenase domain may be derived from the same species of CDH protein (e.g. from the same CDH gene).

Alternatively, in a non-naturally occurring polypeptide having a CDH-heme domain, a CBM, and a dehydrogenase domain, the CDH-heme domain, the CBM, and the dehydrogenase domain are not derived from the same species of CDH protein. In one format, the CDH-heme domain and the dehydrogenase domain are derived from the same species of CDH protein, and the CBM is derived from a non-CDH protein. In another format, the CDH-heme domain, the CBM, and the dehydrogenase domain are each derived from different species of CDH proteins (e.g. from three different CDH genes). In another format, the CDH-heme domain and the CBM are derived from the same species of CDH protein, and the dehydrogenase domain is derived from a non-CDH protein.

In a non-naturally occurring polypeptide having a CDH-heme domain, a CBM, and a dehydrogenase domain, the CDH-heme domain and CBM may be derived from N. crassa CDH-1 (SEQ ID NO: 70 and SEQ ID NO: 74, respectively), and the dehydrogenase domain may be derived from a non-CDH protein. In another format, the CDH-heme domain and CBM are derived from N. crassa CDH-1, and the dehydrogenase domain is derived from a putative glucose/sorbose dehydrogenase from C. cinerea (SEQ ID NO: 51).

In another format, in a non-naturally occurring polypeptide having a CDH-heme domain, a CBM, and a dehydrogenase domain, the CDH-heme domain and CBM may be derived from M. thermophila CDH-1 (SEQ ID NO: 80 and SEQ ID NO: 84), and the dehydrogenase domain may be derived from a non-CDH protein. In another format, the CDH-heme domain and CBM are derived from M. thermophila CDH-1, and the dehydrogenase domain is a putative glucose/sorbose dehydrogenase from C. cinerea (SEQ ID NO: 51).

In a non-naturally occurring polypeptide having a CDH-heme domain, a CBM, and a dehydrogenase domain, the CDH-heme domain and the dehydrogenase domain may be derived from the same species of CDH protein that naturally lacks a CBM, and the CBM may be derived from either a CDH or a non-CDH protein. In one aspect, in a non-naturally occurring polypeptide having a CDH-heme domain, a CBM, and a dehydrogenase domain, the CDH-heme domain and the dehydrogenase domain are derived from N. crassa CDH-2, and the CBM is derived from either a CDH or a non-CDH protein. In another aspect, in a non-naturally occurring polypeptide having a CDH-heme domain, a CBM, and a dehydrogenase domain, the CDH-heme domain and the dehydrogenase domain are derived from N. crassa CDH-2, and the CBM is derived from either a CDH or a non-CDH protein. In another aspect, in a non-naturally occurring polypeptide having a CDH-heme domain, a CBM, and a dehydrogenase domain, the CDH-heme domain and the dehydrogenase domain are derived from M. thermophila CDH-2, and the CBM is derived from N. crassa or M. thermophila CDH-1 protein.

In one format, in a non-naturally occurring polypeptide having a CDH-heme domain, a CBM, and a dehydrogenase domain, the CDH-heme domain and the dehydrogenase domain are derived from N. crassa CDH-2 (SEQ ID NO: 76 and SEQ ID NO: 78, respectively) and the CBM is derived from N. crassa or M. thermophila CDH-1 protein (SEQ ID NO: 74 or SEQ ID NO: 84, respectively).

In another format, in a non-naturally occurring polypeptide having a CDH-heme domain, a CBM, and a dehydrogenase domain, the CDH-heme domain and the dehydrogenase domain are derived from M. thermophila CDH-2 (SEQ ID NO: 86 and SEQ ID NO: 88, respectively) and the CBM is derived from N. crassa or M. thermophila CDH-1 protein (SEQ ID NO: 74 or SEQ ID NO: 84, respectively).

A non-naturally occurring CDH-heme domain polypeptide of the present disclosure may further include any additional polypeptide sequence. Non-naturally occurring CDH-heme domain polypeptide of the present disclosure may additionally include, without limitation, a signal peptide for secretion of the polypeptide, and/or a polypeptide “tag” for protein purification.

A composition containing a CDH-heme domain and a CBM, wherein the CDH-heme domain and the CBM are not part of the same polypeptide chain and are not covalently linked, but they stably interact through non-covalent interactions is also provided. A CDH-heme domain and a CBM that are not part of the same polypeptide chain may be on two separate polypeptides which stably interact non-covalently, for example, through a leucine zipper motif.

Leucine zipper motifs are well-known to one of skill in the art, and are common structures involved in the dimerization of polypeptides. Leucine zipper motifs have leucine resides at about every seventh amino acid in the motif, and form alpha helices, through which the two dimerization partners interact.

### GH61 Polypeptides

Recombinant GH61 polypeptides are also provided herein. Examples of recombinant GH61 polypeptides of the disclosure are polypeptides having the amino acid sequence of GH61-1/NCU02240 (SEQ ID NO: 24), GH61-2/NCU07898 (SEQ ID NO: 26), GH61-4/NCU01050 (SEQ ID NO: 30), GH61-5/NCU08760 (SEQ ID NO: 28), NCU02916 (SEQ ID NO: 64), NCU00836 (SEQ ID NO: 90), or subsequences thereof.

The disclosure provides for a recombinant polypeptide having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity/sequence similarity to a polypeptide of SEQ ID NO: 24 (GH61-1/NCU02240), SEQ ID NO: 26 (GH61-2/NCU07898), SEQ ID NO: 28 (GH61-5/NCU08760), SEQ ID NO: 30 (GH61-4/NCU01050), NCU00836 (SEQ ID NO: 90), or SEQ ID NO: 64 (NCU02916).

GH61 polypeptides of the disclosure also include recombinant polypeptides that are conservatively modified variants of polypeptides of GH61-1/NCU02240, GH61-2/NCU07898, GH61-4/NCU01050, GH61-5/NCU08760, NCU00836, and NCU02916. “Conservatively modified variants” as used herein include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain examples of amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

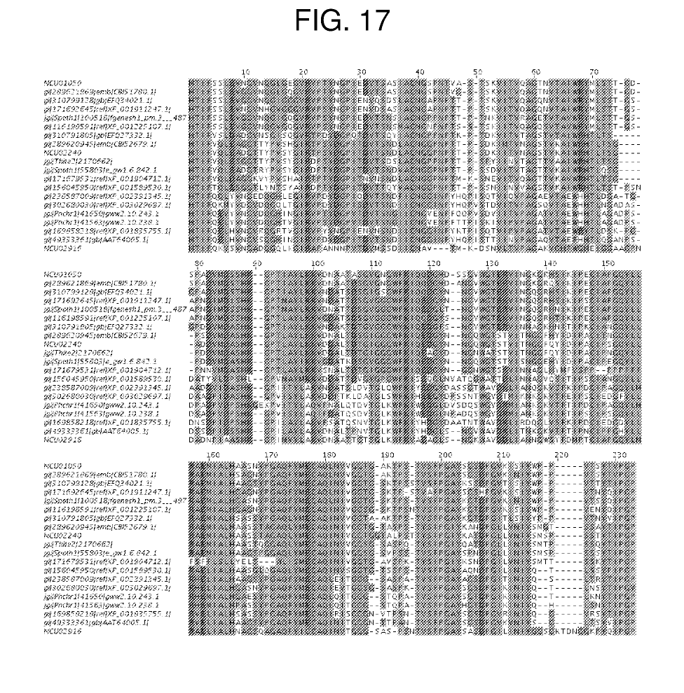

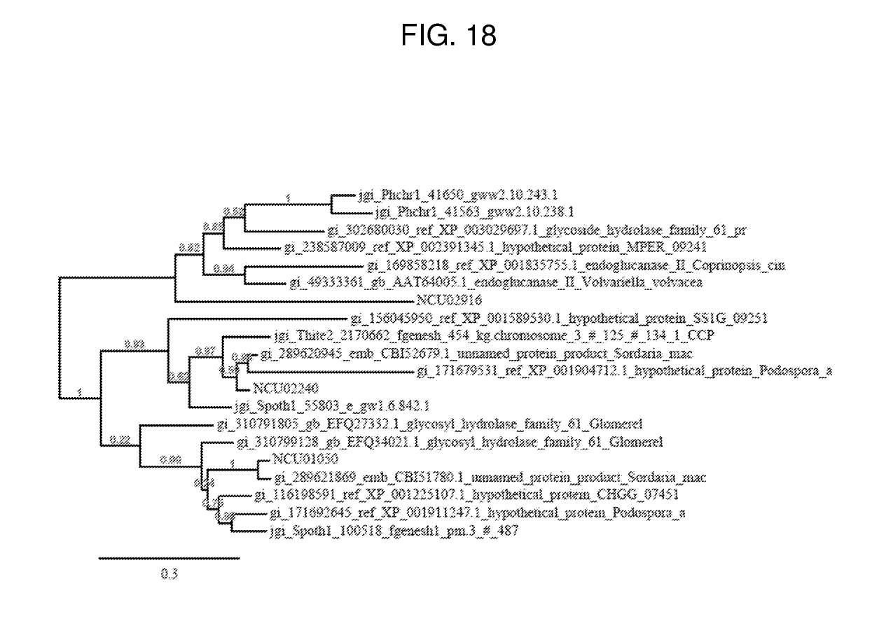

The disclosure provides for GH61 polypeptides homologous or orthologous to NCU02240 or NCU01050. A sequence alignment of polypeptides with homology to NCU02240 or NCU01050 is provided in FIG. 17, and FIG. 18 shows a maximum likelihood phylogeny of selected GH61 proteins to NCU02240 or NCU01050.

Proteins that share certain distinguishing motifs with the polypeptides of NCU02240 and NCU01050 may be referred to as belonging to the “NCU02240/NCU01050 clade.” Proteins that are members of the NCU02240/NCU01050 clade may be identified by comparing a reference NCU02240 or NCU01050 sequence to a second sequence, such as by a BLAST sequence alignment, and by identifying motifs in the second sequence.

As provided herein, GH61 polypeptides that belong to the “NCU02240/NCU0150 clade” have 3 or more, 4 or more, 5 or more, 6 or more, or all 7 of the following motifs in the polypeptide sequence:

Motif 1: HTIF (SEQ ID NO: 34); (corresponds to residues 1-4 of the NCU02240 polypeptide after the signal sequence is cleaved)

Motif 2: R-X-P-[ST]-Y-[ND]-G-P (SEQ ID NO: 35); (corresponds to residues 21-28 of the NCU02240 polypeptide after the signal sequence is cleaved); wherein X is any amino acid, [ST] is S or T, and [ND] is N or D.

Motif 3: C-N-G-X-P-N-[PT]-[TV] (SEQ ID NO: 36); (corresponds to residues 39-46 of the NCU02240 polypeptide after the signal sequence is cleaved); wherein X is any amino acid, [PT] is P or T, and [TV] is T or V.

Motif 4: D-X-X-D-X-[ST]-H-K-G-P-[TV]-X-A-Y-[LM]-K-K-V (SEQ ID NO: 37); (corresponds to residues 75-92 of the NCU02240 polypeptide after the signal sequence is cleaved); wherein X is any amino acid, [ST] is S or T, [TV] is T or V, and [LM] is L or M. Without being bound by theory, the histidine in this motif is known from structural characterizations in the literature to bind an essential metal ion.

Motif 5: G-W-[FY]-K-I-[QS] (SEQ ID NO: 38); (corresponds to residues 104-109 of the NCU02240 polypeptide after the signal sequence is cleaved); wherein [FY] is F or Y and [QS] is Q or S. Without being bound by theory, these residues are far away from the predicted active site and are believed to be important for structural stability of the NCU02240/NCU01050 clade.

Motif 6: I-P-X-C-I-X-X-G-Q-Y-L-L-R-[AG]-E-[ML]-[IL]A-L-H-X-A-X-X-X-X-G-A-Q-[FL]-Y-M-E-C-A-Q-[IL]-N-[IV]-V-G-G (SEQ ID NO: 39); (corresponds to residues 134-177 of the NCU02240 polypeptide after the signal sequence is cleaved); wherein X is any amino acid, [AG] is A or G, [ML] is M or L, [IL] is I or L, [FL] is F or L, [IL] is I or L, and [IV] is I or V. The first cysteine in the motif is in a disulfide bond. The histidine in the motif is near the predicted active site and is highly conserved in nearly all GH61s. The middle glutamine in the motif is absolutely conserved in all GH61 proteins and is known to be important for activity from the literature. The second tyrosine in the motif is very close to the essential active site metal and is also highly conserved across many GH61 clades.

Motif 7: T-[VY]-S-[FI]-P-G-[AI]-Y-X-X-X-D-P-G-X-X-X-X-[IL]-Y (SEQ ID NO: 40); (corresponds to residues 185-204 of the NCU02240 polypeptide after the signal sequence is cleaved); wherein X is any amino acid, [VY] is V or Y, [FI] is F or I, and [AI] is A or I. Without being bound by theory, the last tyrosine in the motif (at the final position) is believed to be important for substrate binding.

In the above motifs, the accepted IUPAC single letter amino acid abbreviation is employed.

Examples of GH61 polypeptides that are members of the “NCU02240/NCU01050 clade” include, without limitation, the polypeptides of SEQ ID NOs: 24, 30, 52, 53, 54, 55, 56, 57, 60 63, 66, 68, and 69.

The present disclosure further provides for conservatively modified variants of GH61 polypeptides that are members of the NCU02240/NCU01050 clade.

GH61 polypeptides disclosed herein include polypeptides containing the motif H-X₍₄₋₈₎-Q-X-Y (SEQ ID NO: 92), wherein X is any amino acid, and X₍₄₋₈₎ is any number from 4 to 8. The H of this motif corresponds to residue 153 of the NCU02240 polypeptide after the signal sequence is cleaved. Without being bound by theory, the H, Q, and Y residues of this motif may be important for binding copper, substrate binding/positioning, and/or acting as a general acid. Mutation of any of the H, Q, and Y residues resides of this motif in a GH61 polypeptide may significantly impair the function of the GH61 polypeptide.

GH61 polypeptides of the disclosure includes both the full-length cDNA translated version of GH61 polypeptide sequence, as well as the corresponding GH61 polypeptide sequence that lacks a signal peptide. When first translated in the cell, all GH61 polypeptides of the disclosure have a short N-terminal signal peptide which targets the polypeptide for extracellular secretion. This polypeptide is cleaved from the original translated GH61 polypeptide when the GH61 polypeptide is transported out of the cell.

Methods for identification of signal peptides on GH61 polypeptide are known in the art, such as by using the SignalP prediction tool. See, for example, “Locating proteins in the cell using TargetP, SignalP, and related tools” Olof Emanuelsson, Søren Brunak, Gunnar von Heijne, Henrik Nielsen Nature Protocols 2, 953-971 (2007).

Manual verification of the predicted signal peptide should show that all mature GH61 polypeptides contain an N-terminal histidine following signal peptide cleavage. If the SignalP predicted N-terminal residue is not histidine, manual prediction of the GH61 should be performed and this can be done by looking for a histidine residue approximately 10-30 amino acids from the N-terminus and commonly 15-25 amino acids from the N-terminus.

This histidine is required for metal binding and ligates the catalytically required metal via the imidazole side chain and N-terminal amine. Hence, any GH61 sequence lacking an N-terminal histidine due to its deletion (or extra sequence on the N-terminus due to an improper signal cleavage event) is rendered nonfunctional.

The signal peptide constitutes amino acid numbers 1-15 of SEQ ID: 24 (NCU02240), amino acid numbers 1-15 of SEQ ID NO: 26 (NCU07898), amino acid numbers 1-20 of SEQ ID NO: 28 (NCU08760), amino acid numbers 1-15 of SEQ ID NO: 30 (NCU01050), amino acid numbers 1-16 of SEQ ID NO: 64 (NCU02916) and amino acid numbers 1-18 of SEQ ID NO: 90 (NCU00836).

Provided herein are GH61 polypeptides of the NCU02240/NCU01050 clade and GH61 polypeptides NCU02240, NCU07898, NCU08760, NCU01050, NCU02916 and NCU00836 having the signal peptide intact. Also provided herein are GH61 polypeptides of the NCU02240/NCU01050 clade and GH61 polypeptides NCU02240, NCU07898, NCU08760, NCU01050, NCU02916 and NCU00836 lacking the signal peptide.

GH61 Polypeptides Bound to Copper

Provided herein are GH61 polypeptides that are bound to a copper atom. GH61 polypeptides that may bind copper atoms include, without limitation, GH61-1/NCU02240, GH61-2/NCU07898, GH61-4/NCU01050, GH61-5/NCU08760, GH61-6/NCU02916, and GH61-3/NCU00836.

Also provided herein are compositions that contain multiple recombinant GH61 polypeptides, wherein 50% or more of the GH61 proteins are bound to a copper atom. Further provided herein are compositions that contain multiple recombinant GH61 polypeptides, wherein 55%, 60%, 65%, 70%, 75%, 80%, 85%, 90%, 91%, 92%, 93%, 94%, 95%, 96%, 97%, 98%, 99%, or more, or 100% of the GH61 proteins are bound to a copper atom.

Compositions that contain multiple recombinant GH61 polypeptides, wherein the ratio of copper atoms to GH61 proteins in the composition is 0.5 to 1 (i.e. 1 copper atom per 2 GH61 proteins) or higher are also provided. In one format, compositions are provided that contain multiple recombinant GH61 polypeptides, wherein the ratio of copper atoms to GH61 proteins in the composition is 0.6, 0.7, 0.8, 0.9, 1 (i.e. 1 copper atom per 1 GH61 protein), 1.1, 1.2, 1.3, 1.4, 1.5, 1.6, 1.7, 1.8, 1.9, 2, 3, 4, 5, 6, 7, 8, 9, 10 (i.e. 10 copper atoms per 1 GH61 protein), or higher, to 1. In compositions wherein the ratio of copper atoms to GH61 proteins is above 1, at least some copper atoms in the composition are not bound to a GH61 protein. Without being bound by theory, a single copper atom may be stably bound by each GH61 protein.

Polynucleotides of the Disclosure

As used herein, the terms “polynucleotide,” “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications. As used herein, the symbols for nucleotides and polynucleotides are those recommended by the IUPAC-IUB Commission of Biochemical Nomenclature.

Polynucleotides of the disclosure are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature [e.g., in Matteucci et al., (1980) Tetrahedron Lett 21:719-722; U.S. Pat. Nos. 4,500,707; 5,436,327; and 5,700,637]. Polynucleotide cloning techniques are well known in the art, and are described, for example in Sambrook, J. et al. 2000 Molecular Cloning: A Laboratory Manual (Third Edition). Briefly, polynucleotide cloning techniques include, without limitation, amplification of polynucleotides by polymerase chain reaction (PCR), enzymatic cleavage of polynucleotides by restriction enzymes, and enzymatic joining of polynucleotides by ligases. Polynucleotide of the disclosure may be prepared by one or any combination of techniques.

Each polynucleotide of the disclosure can be incorporated into an expression vector. “Expression vector” or “vector” refers to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also contains materials to aid in achieving entry of the nucleic acid into the host cell, such as a virus, liposome, protein coating, or the like. The expression vectors contemplated for use in the present disclosure include those into which a nucleic acid sequence can be inserted, along with any preferred or required operational elements. Further, the expression vector must be one that can be transferred into a host cell and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

Incorporation of the individual polynucleotides into vectors may be accomplished through known methods that include, for example, the use of restriction enzymes (such as BamHI, EcoRI, HhaI, XhoI, XmaI, and so forth) to cleave specific sites in the expression vector, e.g., plasmid. The restriction enzyme produces single stranded ends that may be annealed to a polynucleotide having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired polynucleotide are often cleaved with the same restriction enzyme, thereby assuring that the ends of the expression vector and the ends of the polynucleotide are complementary to each other. In addition, DNA linkers maybe used to facilitate linking of nucleic acids sequences into an expression vector.

The disclosure is not limited with respect to the process by which the polynucleotide is incorporated into the expression vector. Those of ordinary skill in the art are familiar with the necessary steps for incorporating a polynucleotide into an expression vector. A typical expression vector contains the desired polynucleotide preceded by one or more regulatory regions, along with a ribosome binding site, e.g., a nucleotide sequence that is 3-9 nucleotides in length and located 3-11 nucleotides upstream of the initiation codon in E. coli. See Shine and Dalgarno (1975) Nature 254(5495):34-38 and Steitz (1979) Biological Regulation and Development (ed. Goldberger, R. F.), 1:349-399 (Plenum, New York).

The term “operably linked” as used herein refers to a configuration in which a control sequence is placed at an appropriate position relative to the coding sequence of the DNA sequence or polynucleotide such that the control sequence directs the expression of the coding sequence.

Regulatory regions include, for example, those regions that contain a promoter and an operator. A promoter is operably linked to the desired polynucleotide, thereby initiating transcription of the polynucleotide via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. Examples include lactose promoters (Lad repressor protein changes conformation when contacted with lactose, thereby preventing the Lad repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Another example is the tac promoter (see de Boer et al., (1983) Proc Natl Acad Sci USA 80(1):21-25). As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC1O1, pBR322, pBBR1MCS-3, pUR, pEX, pMR1OO, pCR4, pBAD24, pUC19; bacteriophages, such as M1 3 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

“Recombinant nucleic acid” or “heterologous nucleic acid” or “recombinant polynucleotide”, “recombinant nucleotide” or “recombinant DNA” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids contains two or more subsequences that are not found in the same relationship to each other in nature. In one aspect, the present disclosure describes the introduction of an expression vector into a host cell, wherein the expression vector contains a nucleic acid sequence coding for a protein that is not normally found in a host cell or contains a nucleic acid coding for a protein that is normally found in a cell but is under the control of different regulatory sequences. With reference to the host cell's genome, then, the nucleic acid sequence that codes for the protein is recombinant.

The relationship between polypeptide sequences and polynucleotide sequences are well known in the art. Amino acids are encoded by a ‘codon’ of three nucleic acids; the codons that encode each nucleic acid are provided, for example, in J M Berg, J L Tymoczko, and L Stryer, Biochemistry, 5^{th} edition (2002). Accordingly, it is routine for one having skill in the art to identify or generate a polynucleotide sequence encoding a polypeptide sequence of interest. Some amino acids are encoded by more than one codon. In polynucleotides of the present disclosure, any sequence of nucleic acids (any codon) that encodes a desired amino acid may be used in the polynucleotide sequence. In some aspects, certain codons are used that have a preferred utilization in a host organism over other codons encoding the same amino acid.

### Polynucleotide Sequences Encoding CDH Heme Domain Polypeptides

Recombinant polynucleotides encoding CDH-heme domain polypeptides are provided herein. Recombinant polynucleotides of the disclosure may be prepared by any method disclosed herein for the preparation of polynucleotides.

The present disclosure includes any recombinant polynucleotide encoding a CDH-heme domain polypeptide. In one format, the present disclosure includes any recombinant polynucleotide encoding a non-naturally occurring CDH-heme domain polypeptide. In one format, a recombinant polynucleotide of the disclosure encodes a non-naturally occurring CDH-heme domain polypeptide including a CDH-heme domain and a CBM, but not a dehydrogenase domain. In one format, a recombinant polynucleotide of the disclosure encodes a non-naturally occurring CDH-heme domain polypeptide including a CDH-heme domain, a CBM, and a dehydrogenase domain.

Polynucleotides encoding CDH heme domain polypeptides include SEQ ID NOs: 33 (N. crassa CDH-1), 42 (N. crassa CDH-2), 45 (M. thermophila CDH-1), 48 (M. thermophila CDH-2), 71 (N. crassa CDH-1 heme domain), 77 (N. crassa CDH-2 heme domain), 81 (M. thermophila CDH-1), and 86 (M. thermophila CDH-2).

### Polynucleotides Encoding GH61 Polypeptides

The present disclosure includes recombinant polynucleotides encoding GH61 polypeptides. Recombinant polynucleotides of the disclosure include any polynucleotide that encodes a GH61 polypeptide disclosed herein. Recombinant polynucleotides encoding a GH61 polypeptide may be prepared by any method disclosed herein for the preparation of polynucleotides.

Polynucleotides of the disclosure include polynucleotides that encode a polypeptide of SEQ ID NO: 24 (GH61-1/NCU02240), SEQ ID NO: 26 (GH61-2/NCU07898), SEQ ID NO: 30 (GH61-4/NCU01050), SEQ ID NO: 28 (GH61-5/NCU08760), SEQ ID NO: 64 (NCU02916) or SEQ ID NO: 90 (NCU00836). Polynucleotides of the disclosure also include the polynucleotides of: SEQ ID NO: 25 (encodes GH61-1/NCU02240 polypeptide), SEQ ID NO: 27 (encodes GH61-2/NCU07898 polypeptide), SEQ ID NO: 31 (encodes GH61-4/NCU01050 polypeptide), SEQ ID NO: 29 (encodes GH61-5/NCU08760 polypeptide) and SEQ ID NO: 91 (encodes NCU00836 polypeptide).

Recombinant polynucleotides of the disclosure also include polynucleotides having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity/sequence similarity to the polynucleotide of SEQ ID NO: 25, SEQ ID NO: 27, SEQ ID NO: 31, SEQ ID NO: 29, and SEQ ID NO: 91.

Polynucleotides of the disclosure further include polynucleotides that encode GH61 polypeptides that are members of the NCU02240/NCU01050 clade. Polynucleotides of the disclosure also include polynucleotides that encode GH61 polypeptides containing the motif H-X₍₄₋₈₎-Q-X-Y.

Polynucleotides of the disclosure further include polynucleotides that encode conservatively modified variants of polypeptides of GH61-1/NCU02240, GH61-2/NCU07898, GH61-4/NCU01050, GH61-5/NCU08760, NCU02916, NCU00836, and polynucleotides that encode conservatively modified variants of GH61 proteins of the NCU02240/NCU01050 clade.

Polynucleotides encoding GH61 polypeptides of the NCU02240/NCU01050 clade and GH61 polypeptides NCU02240, NCU07898, NCU08760, NCU01050, NCU02916 and NCU00836 that have a signal peptide intact are provided.

Polynucleotides encoding GH61 polypeptides of the NCU02240/NCU01050 clade and GH61 polypeptides NCU02240, NCU07898, NCU08760, NCU01050, NCU02916 and NCU00836 that lack a signal peptide intact are also provided.

### Expression of Recombinant Polypeptides of the Disclosure and Host Cells of the Disclosure

The disclosure further provides for the expression of polypeptides of the disclosure. Polypeptides of the disclosure may be prepared by standard molecular biology techniques such as those described in Sambrook, J. et al. 2000 Molecular Cloning: A Laboratory Manual (Third Edition). Recombinant polypeptides may be expressed in and purified from transgenic expression systems. Transgenic expression systems can be prokaryotic or eukaryotic. In some aspects, transgenic host cells may secrete the polypeptide out of the host cell. In some aspects, transgenic host cells may retain the expressed polypeptide in the host cell.

Recombinant polypeptides of the disclosure may be partially or substantially isolated from a host cell, or from the growth media of the host cell. Recombinant polypeptide of the disclosure may be prepared with a protein “tag” to facilitate protein purification, such as a GST-tag or poly-His tag. A recombinant polypeptide of the disclosure may also prepared with a signal sequence to direct the export of the polypeptide out of the cell. Recombinant polypeptides may be only partially purified (e.g. <80% pure, <70% pure, <60% pure, <50% pure, <40% pure, <30% pure, <20% pure, <10% pure, <5% pure), or may be purified to a high degree of purity (e.g. >99% pure, >98% pure, >95% pure, >90% pure, etc.). Recombinant polypeptides may be purified through a variety of techniques known to those of skill in the art, including for example, ion-exchange chromatography, size exclusion chromatography, and affinity chromatography.

The present disclosure further relates to host cells containing recombinant polynucleotides encoding one or more polypeptides of the disclosure. A host cell may contain one or more polynucleotides encoding one or more CDH-heme domain polypeptides and/or one or more polynucleotides encoding one or more recombinant GH61 polypeptides.

Host cells containing a recombinant polynucleotides encoding a polypeptide having the amino acid sequence of GH61-1/NCU02240 (SEQ ID NO: 24), GH61-2/NCU07898 (SEQ ID NO: 26), GH61-4/NCU01050 (SEQ ID NO: 30), GH61-5/NCU08760 (SEQ ID NO: 28), NCU02916 (SEQ ID NO: 64), NCU00836 (SEQ ID NO: 90), N. crassa CDH-1 (SEQ ID NO: 32) or M. thermophila CDH-1 (SEQ ID NO: 46) are provided. Also provided herein are host cells containing two or more recombinant polynucleotides encoding one or more polypeptide having the amino acid sequence of GH61-1/NCU02240, GH61-2/NCU07898, GH61-4/NCU01050, GH61-5/NCU08760, NCU02916, or NCU00836 and one or more polypeptides having the amino acid sequence of N. crassa CDH-1 or M. thermophila CDH-1.

“Host cell” and “host microorganism” are used interchangeably herein to refer to a living biological cell that can be transformed via insertion of recombinant DNA or RNA. Such recombinant DNA or RNA can be in an expression vector. A host organism or cell as described herein may be a prokaryotic organism or a eukaryotic cell.

Any prokaryotic or eukaryotic host cell may be used in the present disclosure so long as it remains viable after being transformed with a sequence of nucleic acids. Preferably, the host cell is not adversely affected by the transduction of the necessary nucleic acid sequences, the subsequent expression of the proteins (e.g., transporters), or the resulting intermediates. Suitable eukaryotic cells include, but are not limited to, fungal, plant, insect or mammalian cells.

The host cell may be a fungal strain. “Fungi” as used herein includes the phyla Ascomycota, Basidiomycota, Chytridiomycota, and Zygomycota as well as the Oomycota and all mitosporic fungi. The host cell may be a yeast cell, including a Candida, Hansenula, Kluyveromyces, Myceliophthora, Neurospora, Pichia, Saccharomyces, Schizosaccharomyces, Trichoderma or Yarrowia strain.

Alternatively, the host cell may be prokaryotic, and in certain aspects, the prokaryotes are E. coli, Bacillus subtilis, Zymomonas mobilis, Clostridium sp., Clostridium phytofermentans, Clostridium thermocellum, Clostridium beijerinckii, Clostridium acetobutylicum (Moorella thermoacetica), Thermoanaerobacterium saccharolyticum, or Klebsiella oxytoca.

Host cells of the present disclosure may be genetically modified in that recombinant nucleic acids have been introduced into the host cells, and as such the genetically modified host cells do not occur in nature. The suitable host cell is one capable of expressing one or more nucleic acid constructs encoding one or more proteins for different functions.

A host cell may naturally produce a polypeptide encoded by a polynucleotide of the disclosure. The polynucleotide encoding the desired polypeptide may be heterologous to the host cell, or it may be endogenous to the host cell but operatively linked to heterologous promoters and/or control regions which result in the higher expression of the polynucleotide in the host cell. In another format, the host cell does not naturally produce the desired polypeptide, and includes heterologous nucleic acid constructs capable of expressing one or more polynucleotides necessary for producing the polypeptide.

Compositions Including Recombinant CDH-Heme Domain Polypeptides and/or Recombinant GH61 Polypeptides

Compositions including a recombinant GH61 polypeptide are provided herein. Compositions including a recombinant CDH-heme domain polypeptide are also provided herein. Compositions including both a recombinant GH61 polypeptide and a recombinant CDH-heme domain polypeptide are further provided herein.

A composition of the disclosure may include a recombinant polypeptide having an amino acid sequence of a GH61 polypeptide. In one format, a recombinant polypeptide having an amino acid sequence of a GH61 polypeptide of the composition contains the motif H-X₍₄₋₈₎-Q-X-Y. In one format, a recombinant polypeptide having an amino acid sequence of a GH61 polypeptide of the composition is of the NCU02240/NCU01050 clade. In one format, a recombinant polypeptide having an amino acid sequence of a GH61 polypeptide of the composition has an amino acid sequence of GH61-1/NCU02240 or GH61-4/NCU01050. In one format, a recombinant polypeptide having an amino acid sequence of a GH61 polypeptide of the composition has an amino acid sequence of GH61-2/NCU07898, GH61-5/NCU08760, NCU02916, or NCU00836.

A composition of the disclosure may include a non-naturally occurring CDH-heme domain polypeptide. In one format, a non-naturally occurring CDH-heme domain polypeptide of the composition may contain a CBM. In one format, a non-naturally occurring CDH-heme domain polypeptide of the composition may contain a CBM and lack a dehydrogenase domain. In one format, a non-naturally occurring CDH-heme domain polypeptide of the composition may contain a CBM and a dehydrogenase domain.

Compositions of the disclosure may include a recombinant polypeptide having an amino acid sequence of GH61-1/NCU02240, GH61-2/NCU07898, GH61-4/NCU01050, GH61-5/NCU08760, NCU02916, NCU00836, and a recombinant CDH-heme domain polypeptide.

Compositions including two or more recombinant polypeptides having an amino acid sequence of GH61-1/NCU02240, GH61-2/NCU07898, GH61-4/NCU01050, GH61-5/NCU08760, NCU02916, and NCU00836, and a recombinant CDH-heme domain polypeptide are provided herein.

A composition including a recombinant GH61 polypeptide and a recombinant CDH-heme domain polypeptide containing a CBM is provided herein. In one format, the recombinant CDH-heme domain polypeptide of the composition has the amino acid sequence of a naturally occurring CDH protein. In one format, the recombinant CDH-heme domain polypeptide of the composition has the amino acid sequence of N. crassa CDH-1 or M. thermophila CDH-1. In another format, the recombinant CDH-heme domain polypeptide of the composition lacks a dehydrogenase domain and a CBM.

A composition including a recombinant GH61 polypeptide and two or more recombinant CDH-heme domain polypeptides, wherein the at least one of the two or more recombinant CDH-heme domain polypeptides lacks a dehydrogenase domain and a CBM is also provided herein.

Another composition of the disclosure includes a recombinant GH61 polypeptide and a non-naturally occurring CDH-heme domain polypeptide. In some formats, these compositions contain two or more non-naturally occurring CDH-heme domain polypeptides.

Compositions of the disclosure also include compositions including a recombinant GH61 polypeptide and a non-naturally occurring CDH-heme domain polypeptide, wherein the non-naturally occurring CDH-heme domain polypeptide contains a CDH-heme domain and a CBM, but lacks a dehydrogenase domain.

Compositions of the disclosure also include compositions including a recombinant GH61 polypeptide and a non-naturally occurring CDH-heme domain polypeptide, wherein the non-naturally occurring CDH-heme domain polypeptide contains a CDH-heme domain, a CBM, and a dehydrogenase domain.

Compositions including a recombinant GH61 polypeptide and a recombinant CDH-heme domain polypeptide may further include one or more cellulase enzymes.

Compositions of the disclosure also include compositions including a recombinant GH61 polypeptide and a CDH-heme domain polypeptide covalently joined as a single polypeptide chain. Such compositions may further include one or more cellulase enzymes.

### Cellulases

Cellulases are enzymes that can hydrolyze cellulose. They include, but are not limited to, exoglucanases (cellobiohydrolases), endoglucanases, and β-glucosidases. Cellulases are naturally produced by many different organisms, primarily species of fungi and bacteria.

Endoglucanases hydrolyze internal 1-4 β-glycosidic linkages in cellulose, thereby reducing the length of cellulose polymers and increasing the amount of exposed ends of the cellulose polymers. Examples of endoglucanases include, without limitation, the polypeptides of EGI/Cel7B, EGII/Cel5A, EGIII/Cel12A, EGIV/Cel61A and EGV/Cel45A from Trichoderma reesei (“T. reesei”), the polypeptides of EG28, EG34, and EG44 from Phanerochaete chrysosporium (“P. chrysosporium”), and the polypeptides of NCU00762, NCU05057, and NCU07190 from Neurospora crassa (“N. crassa”).

Exoglucanases hydrolyze 1-4 β-glycosidic linkages near the end of the cellulose polymers, thereby generating short chains of cellulose-derived glucose polymers, referred to as “cellodextrins”. The most commonly generated cellodextrin is “cellobiose” (2 glucose molecules), but longer cellodextrins may be generated as well, including cellotrioses (3 glucose molecules), cellotetraoses (4 glucose molecules), cellopentaoses (5 glucose molecules), cellohexaoses (6 glucose molecules), and longer. Examples of exoglucanases include, without the limitation, the polypeptides of CBHII/Cel6A and CBHI/Cel7A of T. reesei, and the polypeptides of NCU07340 and NCU09680 of N. crassa.

β-glucosidases hydrolyze cellodextrins to glucose. Examples of β-glucosidases include, without limitation, the polypeptides of TRBLG2 of T. reesei, CCBGLA of Clostridium cellulovorans, GH3-4/NCU04952 of N. crassa and NKBL1 of Neotermes koshunensis.

Cellulases of the present disclosure include both naturally occurring cellulases, and cellulases that have been engineered to have improved properties (e.g. improved catalytic rate, improved thermostability, etc.). In one aspect, provided herein is a composition of cellulases that includes at least 1 endoglucanase, at least 1 exoglucanase, and at least one β-glucosidase.

Examples of organisms from which cellulases may be purified from, and/or from which genes encoding cellulases may be cloned from, include, without limitation, fungi: Aspergillus niger, Aspergillus oryzae, Chaetomium globosum, Chaetomium thermophilum, Formitopsis palustris, Humicola insolens, Myceliophthora thermophila, Neurospora crassa, Penicillium spp., Phanerochaete chrysosporium, Pisolithus tinctorius, Pleurotus ostreatus, Podospora anserine, Postia placenta, Saccharomyces cerevisiae, Sporotrichum thermophile, Sporobolomyces singularis, Talaromyces emersonii, Thielavia terrestris, Trametes versicolor, Trichoderma reesei (teleomorph: Hypocrea jecorina); and bacteria: Acidothermus cellulolyticus, Anaerocellum thermophilum, Bacillus pumilis, Caldibacillus cellovorans, Caldicellulosiruptor saccharolyticum, Clostridium thermocellum, Halocella cellulolytica, Streptomyces reticule, Thermotoga neapolitana.

Compositions are provided herein including one or more non-naturally occurring CDH-heme domain polypeptides and one or more cellulase enzymes. Also provided herein are compositions including one or more recombinant GH61 polypeptides of the NCU02240/NCU01050 clade and one or more cellulase enzymes. Also provided herein are compositions including a recombinant polypeptides having an amino acid sequence of NCU02240 or NCU01050, and one or more cellulase enzymes

Compositions of the disclosure also include compositions including one or more non-naturally occurring CDH-heme domain polypeptides, one or more recombinant GH61 polypeptides, and one or more cellulase enzymes.

Compositions are also provided herein including one or more non-naturally occurring CDH-heme domain polypeptides, one or more polypeptides having an amino acid sequence of GH61-1/NCU02240, GH61-2/NCU07898, GH61-4/NCU01050, GH61-5/NCU08760, NCU02916 or NCU00836 and one or more cellulase enzymes.

Compositions are also provided herein including one or more non-naturally occurring CDH-heme domain polypeptides, one or more GH61 polypeptides containing the motif H-X₍₄₋₈₎-Q-X-Y, and one or more cellulase enzymes.

Compositions provided herein including one or more non-naturally occurring CDH-heme domain polypeptides, one or more recombinant GH61 polypeptides, and cellulases are more effective at degrading cellulose-containing materials than otherwise equivalent compositions that contain cellulases but lack the one or more non-naturally occurring CDH-heme domain polypeptides and the one or more recombinant GH61 polypeptides.

### Additional Compositions

Compositions of the disclosure also include compositions including a CDH-heme domain and a CBM, wherein the CDH-heme domain and the CBM are not covalently linked, but they stably interact through non-covalent interactions, and that further contain a GH61 polypeptide.

Also disclosed herein is a composition containing a CDH-heme domain and a CBM, wherein the CDH-heme domain and the CBM are not covalently linked, but are parts of two polypeptides that stably interact through a leucine zipper motif. The composition may further contain a GH61 polypeptide.

Also disclosed herein is a composition containing a CDH-heme domain and a CBM, wherein the CDH-heme domain and the CBM are not covalently linked, but they stably interact through non-covalent interactions, and that further contains one or more polypeptides having an amino acid sequence of GH61-1/NCU02240, GH61-2/NCU07898, GH61-4/NCU01050, GH61-5/NCU08760, NCU02916 or NCU00836.

Also disclosed herein is a composition containing a CDH-heme domain and a CBM, wherein the CDH-heme domain and the CBM are not covalently linked, but they stably interact through non-covalent interactions, and that further contains a GH61 polypeptide and one or more cellulases.

Also provided herein are compositions including one or more recombinant GH61 polypeptides, one or more recombinant CDH-heme domain polypeptides, and culture media from a cellulase-excreting fungus. In such compositions, the one or more recombinant CDH-heme domain polypeptides may be one or more non-naturally occurring CDH-heme domain polypeptides.

Also provided herein are compositions including one or more recombinant GH61 polypeptides, one or more recombinant CDH-heme domain polypeptides, and a composition containing one or more proteins secreted by a cellulase-excreting fungus. In such compositions, the one or more recombinant CDH-heme domain polypeptides may be one or more non-naturally occurring CDH-heme domain polypeptides.

Cellulase-excreting fungi include, but are not limited to, Myceliophthora thermophila, Neurospora crassa, Phanerochaete chrysosporium, and Trichoderma reesei.

Methods

Methods for the degradation of cellulose and cellulose-containing materials such as biomass into monosaccharides and oligosaccharides are provided herein. Additionally, disclosed herein are methods and uses of the polypeptides, polynucleotides, and compositions of the present disclosure for such purposes, for example, in degrading cellulose and cellulose-containing materials to produce soluble sugars.

As used herein, “degrading” and “degradation” of cellulose and cellulose-containing materials refers to any mechanism that results in the depolymerization of cellulose and/or the release of monosaccharides or oligosaccharides from cellulose polysaccharides. Degradation of cellulose includes, without limitation, hydrolysis of cellulose and oxidative cleavage of cellulose.

Methods of Degrading Cellulose

A method of degrading cellulose is provided, wherein the method includes contacting cellulose with one or more cellulases, a recombinant GH61 polypeptide and a recombinant CDH-heme domain polypeptide.

In one aspect, a method of degrading cellulose is provided, wherein the method includes contacting cellulose with one or more cellulases, a recombinant polypeptide having an amino acid sequence of GH61-1/NCU02240, GH61-2/NCU07898, GH61-4/NCU01050, GH61-5/NCU08760, NCU02916, or NCU00836, and a recombinant CDH-heme domain polypeptide.

In another aspect, a method of degrading cellulose is provided, wherein the method includes contacting cellulose with one or more cellulases, a recombinant polypeptide having an amino acid sequence of a polypeptide of the NCU02240/NCU01050 clade, and a recombinant CDH-heme domain polypeptide.

In another aspect, a method of degrading cellulose is provided, wherein the method includes contacting cellulose with one or more cellulases, a recombinant GH61 polypeptide containing the motif H-X₍₄₋₈₎-Q-X-Y, and a non-naturally occurring CDH-heme domain polypeptide.

In another aspect, a method of degrading cellulose is provided, wherein the method includes contacting cellulose with one or more cellulases, two or more recombinant polypeptides having an amino acid sequence of GH61-1/NCU02240, GH61-2/NCU07898, GH61-4/NCU01050, GH61-5/NCU08760, NCU02916, and NCU00836, and a recombinant CDH-heme domain polypeptide.

In another aspect, a method of degrading cellulose is provided, wherein the method includes contacting cellulose with one or more cellulases, a recombinant GH61 polypeptide and a recombinant CDH-heme domain polypeptide containing a CBM.

In another aspect, a method of degrading cellulose is provided, wherein the method includes contacting cellulose with one or more cellulases, a recombinant GH61 polypeptide and a recombinant CDH-heme domain polypeptide having the amino acid sequence of a naturally occurring CDH protein.

In another aspect, a method of degrading cellulose is provided, wherein the method includes contacting cellulose with one or more cellulases, a recombinant GH61 polypeptide, and a recombinant polypeptide of N. crassa CDH-1 or M. thermophila CDH-1.

In another aspect, a method of degrading cellulose is provided, wherein the method includes contacting cellulose with one or more cellulases, a recombinant GH61 polypeptide, and a recombinant CDH-heme domain polypeptide, wherein the recombinant CDH-heme domain polypeptide lacks a dehydrogenase domain and a CBM.

In another aspect, a method of degrading cellulose is provided, wherein the method includes contacting cellulose with one or more cellulases, a recombinant GH61 polypeptide, and two or more recombinant CDH-heme domain polypeptides, wherein the at least one of the two or more recombinant CDH-heme domain polypeptides lacks a dehydrogenase domain and a CBM.

In another aspect, a method of degrading cellulose is provided, wherein the method includes contacting cellulose with one or more cellulases, a recombinant GH61 polypeptide, and a non-naturally occurring CDH-heme domain polypeptide.

In another aspect, a method of degrading cellulose is provided, wherein the method includes contacting cellulose with one or more cellulases, a recombinant GH61 polypeptide, and two or more non-naturally occurring CDH-heme domain polypeptides.

In another aspect, a method of degrading cellulose is provided, wherein the method includes contacting cellulose with one or more cellulases, a recombinant GH61 polypeptide, and a non-naturally occurring CDH-heme domain polypeptide, wherein the non-naturally occurring CDH-heme domain polypeptide contains a CDH-heme domain and a CBM, but lacks a dehydrogenase domain.

In another aspect, a method of degrading cellulose is provided, wherein the method includes contacting cellulose with one or more cellulases, a recombinant GH61 polypeptide and a non-naturally occurring CDH-heme domain polypeptide, wherein the non-naturally occurring CDH-heme domain polypeptide contains a CDH-heme domain, a CBM, and a dehydrogenase domain.

In another aspect, a method of degrading cellulose is provided, wherein the method includes contacting cellulose with a non-naturally occurring CDH-heme domain polypeptide and one or more cellulases.

In another aspect, a method of degrading cellulose is provided, wherein the method includes contacting cellulose with a GH61 polypeptide and one or more cellulases. In one aspect, a method of degrading cellulose is provided, wherein the method includes contacting cellulose with a polypeptide having an amino acid sequence of GH61-1/NCU02240, GH61-2/NCU07898, GH61-4/NCU01050, GH61-5/NCU08760, NCU02916, or NCU00836 and one or more cellulases. In one aspect, a method of degrading cellulose is provided, wherein the method includes contacting cellulose with a polypeptide having an amino acid sequence of a polypeptide of the NCU02240/NCU01050 clade, and one or more cellulases.

In another aspect, a method of degrading cellulose is provided, wherein the method includes contacting the cellulose with a GH61 polypeptide, a molecule containing a heme domain and a CBM, and one or more cellulases. In some aspects, a molecule containing a heme domain may be any molecule containing a heme group capable of transferring electrons.

In another aspect, a method of degrading cellulose is provided, wherein the method includes contacting the cellulose with a Lewis acid, a molecule containing a heme domain and a CBM, and one or more cellulases. In some aspects, a molecule containing a heme domain may be any molecule containing a heme group capable of transferring electrons. A Lewis acid is molecule which is an electron-pair acceptor.

In another aspect, a method of degrading cellulose is provided, wherein the method includes contacting the cellulose with a Lewis acid, a CDH protein having a CBM, and one or more cellulases. A Lewis acid is molecule which is an electron-pair acceptor.

Methods of Increasing the Degradation of Cellulose

A method of increasing degradation of cellulose is provided, wherein the method includes providing a GH61 polypeptide and a CDH-heme domain polypeptide to a reaction mixture containing cellulose and one or more cellulases. In one aspect, a method of increasing degradation of cellulose is provided, wherein the method includes providing a GH61 polypeptide and a non-naturally occurring CDH-heme domain polypeptide to a reaction mixture containing cellulose and one or more cellulases. In another aspect, a method of increasing degradation of cellulose is provided, wherein the method includes providing a polypeptide having an amino acid sequence of GH61-1/NCU02240, GH61-2/NCU07898, GH61-4/NCU01050, GH61-5/NCU08760, NCU02916 or NCU00836, and a CDH-heme domain polypeptide to a reaction mixture containing cellulose and one or more cellulases. In another aspect, a method of increasing degradation of cellulose is provided, wherein the method includes providing a polypeptide having an amino acid sequence of a polypeptide of the NCU02240/NCU01050 clade and a CDH-heme domain polypeptide to a reaction mixture containing cellulose and one or more cellulases. In another aspect, a method of increasing degradation of cellulose is provided, wherein the method includes providing a GH61 polypeptide containing the motif H-X₍₄₋₈₎-Q-X-Y and a CDH-heme domain polypeptide to a reaction mixture containing cellulose and one or more cellulases.

In another aspect, a method of increasing degradation of cellulose is provided, wherein the method includes providing a GH61 polypeptide and a CDH-heme domain polypeptide having a CBM to a reaction mixture containing cellulose and one or more cellulases. In another aspect, a method of increasing degradation of cellulose is provided, wherein the method includes providing a GH61 polypeptide and a non-naturally occurring CDH-heme domain polypeptide having a CBM to a reaction mixture containing cellulose and one or more cellulases.

Degradation of cellulose may be increased to a greater degree by providing a CDH-heme domain polypeptide having a CBM than by providing an equivalent or similar CDH-heme domain polypeptide lacking a CBM. In such examples, the CDH-heme domain polypeptide having a CBM may be non-naturally occurring.

Examples of increasing degradation of cellulose include, without limitation: increasing the rate of degradation of cellulose; increasing the extent of degradation of cellulose; increasing the extent of degradation of cellulose within a certain reaction time; reducing the amount of cellulases necessary to achieve a given extent of degradation of cellulose; and reducing the amount of cellulases necessary to achieve a given extent of degradation of cellulose within a certain reaction time.

In another aspect, a method of increasing degradation of cellulose is provided, wherein the method includes providing a GH61 polypeptide in a reaction mixture including cellulose and one or more cellulases. In another aspect, a method of increasing degradation of cellulose is provided, wherein the method includes providing two or more GH61 polypeptides in a reaction mixture containing cellulose and one or more cellulases. In another aspect, a method of increasing degradation of cellulose is provided, wherein the method includes providing a polypeptide having the amino acid sequence of GH61-1/NCU02240, GH61-2/NCU07898, GH61-4/NCU01050, GH61-5/NCU08760, NCU02916, or NCU00836, in a reaction mixture including cellulose and one or more cellulases. In another aspect, a method of increasing degradation of cellulose is provided, wherein the method includes providing a polypeptide having the amino acid sequence of a polypeptide of the NCU02240/NCU01050 clade in a reaction mixture including cellulose and one or more cellulases. In another aspect, a method of increasing degradation of cellulose is provided, wherein the method includes providing a GH61 polypeptide containing the motif H-X₍₄₋₈₎-Q-X-Y in a reaction mixture including cellulose and one or more cellulases.

A method of degrading cellulose including contacting cellulose with one or more cellulases, a recombinant GH61 polypeptide and a recombinant CDH-heme domain polypeptide may be more effective at degrading cellulose than an otherwise equivalent method that does not include contacting cellulose with a recombinant GH61 polypeptide and/or a recombinant CDH-heme domain polypeptide.

Method of Reducing the Amount of CDH-Heme Domain Polypeptides Necessary to Achieve Increased Degradation of Cellulose

A method of reducing the amount of CDH-heme domain polypeptides necessary to achieve an increased degradation of cellulose is also provided herein, wherein CDH-heme domain polypeptides having a CBM are provided in a reaction mixture including cellulose, cellulases, and a GH61 polypeptide to increase degradation of cellulose, and wherein fewer CDH-heme domain polypeptides having a CBM are required to achieve the increased degradation of cellulose than would be required with a similar or equivalent CDH-heme domain polypeptide lacking a CBM. In such methods, the CDH-heme domain polypeptides may be non-naturally occurring CDH-heme domain polypeptides.

Methods of Reducing Oxidative Damage to Molecules in a Cellulase Reaction

Methods of reducing oxidative damage to molecules in a cellulase reaction and reducing formation of reactive oxygen species in a cellulase reaction are also provided. Molecules in a cellulase reaction include, without limitation, proteins and carbohydrates.

In one aspect, a method of reducing oxidative damage to molecules in a cellulase reaction includes providing a non-naturally occurring CDH-heme domain polypeptide having a CDH-heme domain and a CBM, but lacking a dehydrogenase domain, in a reaction mixture including cellulose, cellulases, and a GH61 polypeptide. A non-naturally occurring CDH-heme domain polypeptide having a CDH-heme domain and a CBM, but lacking a dehydrogenase domain, may generate less oxidative damage to molecules in a cellulase reaction than an equivalent or similar non-naturally occurring CDH-heme domain polypeptide having a CDH-heme domain and a CBM, but having a dehydrogenase domain.

A method of reducing the formation of reactive oxygen species in a cellulase reaction may include providing a non-naturally occurring CDH-heme domain polypeptide having a CDH-heme domain and a CBM, but lacking a dehydrogenase domain, in a reaction mixture including cellulose, cellulases, and a GH61 polypeptide. A non-naturally occurring CDH heme domain polypeptide having a CDH-heme domain and a CBM, but lacking a dehydrogenase domain, may generate fewer reactive oxygen species in a cellulase reaction than an equivalent or similar non-naturally occurring CDH heme domain polypeptide having a CDH-heme domain and a CBM, but having a dehydrogenase domain.

Methods of Degrading Biomass

Methods of degrading biomass are provided. “Biomass” as used herein refers to any material that contains cellulose. Methods disclosed herein relating to cellulose are also applicable to compositions that contain biomass.

Methods of degrading biomass are provided wherein the method includes contacting the biomass with one or more recombinant polypeptides of the current disclosure. In one aspect, a method of degrading biomass is provided, wherein the method includes contacting the biomass with a recombinant CDH-heme domain polypeptide and a recombinant GH61 polypeptide. In another aspect, a method of degrading biomass is provided, wherein the method includes contacting the biomass with a non-naturally occurring CDH-heme domain polypeptide and a GH61 polypeptide. In another aspect, a method of degrading biomass is provided, wherein the method includes contacting the biomass with a CDH-heme domain polypeptide and one or more polypeptides having the amino acid sequences of GH61-1/NCU02240, GH61-2/NCU07898, GH61-4/NCU01050, GH61-5/NCU08760, NCU02916, and NCU00836. In another aspect, a method of degrading biomass is provided, wherein the method includes contacting the biomass with a CDH-heme domain polypeptide and one or more polypeptides having the amino acid sequence of a polypeptide of the NCU02240/NCU01050 clade. In another aspect, a method of degrading biomass is provided, wherein the method includes contacting the biomass with a CDH-heme domain polypeptide and one or more GH61 polypeptides containing the motif H-X₍₄₋₈₎-Q-X-Y.

Biomass suitable for use with the currently disclosed methods include any cellulose-containing material, and include, without limitation, Miscanthus, switchgrass, cord grass, rye grass, reed canary grass, elephant grass, common reed, wheat straw, barley straw, canola straw, oat straw, corn stover, soybean stover, oat hulls, sorghum, rice hulls, rye hulls, wheat hulls, sugarcane bagasse, copra meal, copra pellets, palm kernel meal, corn fiber, Distillers Dried Grains with Solubles (DDGS), Blue Stem, corncobs, pine wood, birch wood, willow wood, aspen wood, poplar wood, energy cane, waste paper, sawdust, forestry wastes, municipal solid waste, waste paper, crop residues, other grasses, and other woods.

Prior to contacting the biomass with one or more polypeptides of the disclosure, biomass may be subjected to one or more pre-processing steps. Pre-processing steps are known to those of skill in the art, and include physical and chemical processes. Pre-processing steps include, without limitation, acid hydrolysis, ammonia fiber expansion (AFEX), sulfite pretreatment to overcome recalcitrance of lignocellulose (SPORL), steam explosion, and ozone pretreatment.

In another aspect, a method of degrading biomass is provided, wherein the method includes contacting biomass with one or more cellulases, a recombinant GH61 polypeptide and a recombinant CDH-heme domain polypeptide.

In another aspect, a method of degrading biomass is provided, wherein the method includes contacting biomass with one or more cellulases, and a composition including a recombinant GH61 polypeptide and a recombinant CDH-heme domain polypeptide.

In another aspect, a method of degrading biomass is provided, wherein the method includes contacting biomass with one or more cellulases, a recombinant polypeptide having an amino acid sequence of GH61-1/NCU02240, GH61-2/NCU07898, GH61-4/NCU01050, GH61-5/NCU08760, NCU02916, or NCU00836, and a recombinant CDH-heme domain polypeptide.

In another aspect, a method of degrading biomass is provided, wherein the method includes contacting biomass with one or more cellulases, a recombinant polypeptide having an amino acid sequence of a polypeptide of the NCU02240/NCU01050 clade, and a recombinant CDH-heme domain polypeptide.

In another aspect, a method of degrading biomass is provided, wherein the method includes contacting biomass with one or more cellulases, a recombinant GH61 polypeptide containing the motif H-X₍₄₋₈₎-Q-X-Y, and a non-naturally occurring CDH-heme domain polypeptide.

In another aspect, a method of degrading biomass is provided, wherein the method includes contacting biomass with one or more cellulases, two or more recombinant polypeptides having amino acid sequences of GH61-1/NCU02240, GH61-2/NCU07898, GH61-4/NCU01050, GH61-5/NCU08760, NCU02916, or NCU00836, and a recombinant CDH-heme domain polypeptide.

In another aspect, a method of degrading biomass is provided, wherein the method includes contacting biomass with one or more cellulases, a recombinant GH61 polypeptide and a recombinant CDH-heme domain polypeptide containing a CBM.

In another aspect, a method of degrading biomass is provided, wherein the method includes contacting biomass with one or more cellulases, a recombinant GH61 polypeptide and a recombinant CDH-heme domain polypeptide having the amino acid sequence of a naturally occurring CDH protein.

In another aspect, a method of degrading biomass is provided, wherein the method includes contacting biomass with one or more cellulases, a recombinant GH61 polypeptide, and a recombinant polypeptide of N. crassa CDH-1 or M. thermophila CDH-1.

In another aspect, a method of degrading biomass is provided, wherein the method includes contacting biomass with one or more cellulases, a recombinant GH61 polypeptide, and a recombinant CDH-heme domain polypeptide, wherein the recombinant CDH-heme domain polypeptide lacks a dehydrogenase domain and a CBM.

In another aspect, a method of degrading biomass is provided, wherein the method includes contacting biomass with one or more cellulases, a recombinant GH61 polypeptide, and two or more recombinant CDH-heme domain polypeptides, wherein the at least one of the two or more recombinant CDH-heme domain polypeptides lacks a dehydrogenase domain and a CBM.

In another aspect, a method of degrading biomass is provided, wherein the method includes contacting biomass with one or more cellulases, a recombinant GH61 polypeptide, and a non-naturally occurring CDH-heme domain polypeptide.

In another aspect, a method of degrading biomass is provided, wherein the method includes contacting biomass with one or more cellulases, a recombinant GH61 polypeptide, and two or more non-naturally occurring CDH-heme domain polypeptides.

In another aspect, a method of degrading biomass is provided, wherein the method includes contacting biomass with one or more cellulases, a recombinant GH61 polypeptide, and a non-naturally occurring CDH-heme domain polypeptide, wherein the non-naturally occurring CDH-heme domain polypeptide contains a CDH-heme domain and a CBM, but lacks a dehydrogenase domain.

In another aspect, a method of degrading biomass is provided, wherein the method includes contacting biomass with one or more cellulases, a recombinant GH61 polypeptide and a non-naturally occurring CDH-heme domain polypeptide, wherein the non-naturally occurring CDH-heme domain polypeptide contains a CDH-heme domain, a CBM, and a dehydrogenase domain.

In another aspect, a method of degrading biomass is provided, wherein the method includes contacting biomass with a non-naturally occurring CDH-heme domain polypeptide and one or more cellulases.

In another aspect, a method of degrading biomass is provided, wherein the method includes contacting biomass with a GH61 polypeptide and one or more cellulases. In another aspect, a method of degrading biomass is provided, wherein the method includes contacting biomass with a polypeptide having an amino acid sequence of GH61-1/NCU02240, GH61-2/NCU07898, GH61-4/NCU01050, GH61-5/NCU08760, NCU02916, or NCU00836, and one or more cellulases. In another aspect, a method of degrading biomass is provided, wherein the method includes contacting biomass with a polypeptide having an amino acid sequence of a polypeptide of the NCU02240/NCU01050 clade and one or more cellulases.

In another aspect, a method of degrading biomass is provided, wherein the method includes contacting the biomass with a GH61 polypeptide, a molecule containing a heme domain, and one or more cellulases. A molecule containing a heme domain may be any molecule containing a heme group capable of transferring electrons.

In another aspect, a method of degrading biomass is provided, wherein the method includes contacting the biomass with a Lewis acid, a molecule containing a heme domain and a CBM, and one or more cellulases. In some aspects, a molecule containing a heme domain may be any an organic molecule containing a heme group capable of transferring electrons. A Lewis acid is molecule which is an electron-pair acceptor.

In another aspect, a method of degrading biomass is provided, wherein the method includes contacting the biomass with a Lewis acid, a CDH protein having a CBM, and one or more cellulases. A Lewis acid is molecule which is an electron-pair acceptor.

In another aspect, a method of degrading biomass is provided, wherein the method includes first contacting biomass with a CDH-heme domain polypeptide and a GH61 polypeptide to create a reaction mixture, and subsequently adding one or more cellulases to the reaction mixture.

Methods of Reducing Oxidative Damage During Degradation of Biomass

A method of reducing oxidative damage to molecules in a reaction involving degradation of biomass is provided, wherein the method includes first contacting biomass with a CDH-heme domain polypeptide and a GH61 polypeptide to create a reaction mixture, and subsequently adding one or more cellulases to the reaction mixture, in order to reduce oxidative damage to molecules in the reaction as compared to the oxidative damage to molecules in the reaction that would occur if the CDH-heme domain polypeptide, the GH61 polypeptide, and the one or more cellulase would be added to the reaction mixture with the biomass at the same time.

Method of Increasing Degradation of Biomass

A method of increasing degradation of biomass is provided, wherein the method includes providing a GH61 polypeptide in a reaction mixture including biomass and one or more cellulases. In one aspect, a method of increasing degradation of biomass is provided, wherein the method includes providing two or more GH61 polypeptides in a reaction mixture containing biomass and one or more cellulases. In another aspect, a method of increasing degradation of biomass is provided, wherein the method includes providing a polypeptide having the amino acid sequence of GH61-1/NCU02240, GH61-2/NCU07898, GH61-4/NCU01050, GH61-5/NCU08760, NCU02916, or NCU00836, in a reaction mixture including biomass and one or more cellulases. In another aspect, a method of increasing degradation of biomass is provided, wherein the method includes providing a polypeptide having the amino acid sequence of a polypeptide of the NCU02240/NCU01050 clade in a reaction mixture including biomass and one or more cellulases.

In one aspect, a method of increasing degradation of biomass is provided, wherein the method includes providing a GH61 polypeptide in a reaction mixture including biomass, one or more cellulases, and an non-naturally occurring CDH-heme domain polypeptide.

Method of Converting Cellulose and Biomass to Fermentation Product

Methods of converting cellulose and biomass to a fermentation product are also provided, wherein cellulose or biomass is contacted with cellulases and one or more polypeptides of the current disclosure, to yield a sugar solution (containing monosaccharides, disaccharides, and oligosaccharides), and the sugars are converted to a fermentation product.

The sugars may be converted into a fermentation product by chemical or microbial fermentation. Fermentative microorganisms include fungi and bacteria species. In one example, the fermentative organism is Saccharomyces cerevisiae.

“Sugars” as used herein includes monosaccharides, disaccharides, and oligosaccharides. In some aspects, sugars are glucose monomers.

Fermentation products of the disclosure include any chemical product that may be produced from sugars obtained by the degradation of cellulose. A fermentation product of the disclosure may be a biofuel. Fermentation products of the disclosure may be alcohols, including but not limited to, ethanol, n-propanol, iso-butanol, 3-methyl-1-butanol, 2-methyl-1-butanol, 3-methyl-1-pentanol, and octanol. A fermentation product of the disclosure may be a ketone or an aldehyde.

Methods of Reducing the Viscosity of Pretreated Biomass Mixtures

The CDH-heme domain polypeptides and GH61 polypeptides provided herein may also be used for pretreating biomass mixtures prior to their degradation into monosaccharides and oligosaccharides, for example, in biofuel production.

Biomass that is used for as a feedstock, for example, in biofuel production, generally contains high levels of lignin, which can block hydrolysis of the cellulosic component of the biomass. Typically, biomass is pretreated with, for example, high temperature and/or high pressure to increase the accessibility of the cellulosic component to hydrolysis. However, pretreatment generally results in a biomass mixture that is highly viscous. The high viscosity of the pretreated biomass mixture can also interfere with effective hydrolysis of the pretreated biomass. Advantageously, the CDH-heme domain polypeptides and GH61 polypeptides of the present disclosure can be used with cellulases to reduce the viscosity of pretreated biomass mixtures prior to further degradation of the biomass. In some aspects, a CDH-heme domain polypeptide of the present disclosure and a GH61 polypeptide having an amino acid sequence of GH61-1/NCU02240, GH61-2/NCU07898, GH61-4/NCU01050, GH61-5/NCU08760, NCU02916, or NCU00836 are used to reduce the viscosity of pretreated biomass mixtures. In some aspects, a CDH-heme domain polypeptide of the present disclosure, a GH61 polypeptide having an amino acid sequence of GH61-1/NCU02240, GH61-2/NCU07898, GH61-4/NCU01050, GH61-5/NCU08760, NCU02916, or NCU00836, and cellulases are used to reduce the viscosity of pretreated biomass mixtures. In some aspects, a non-naturally occurring CDH-heme domain polypeptide of the present disclosure, a GH61 polypeptide containing the motif H-X₍₄₋₈₎-Q-X-Y, and cellulases are used to reduce the viscosity of pretreated biomass mixtures.

Accordingly, certain aspects of the present disclosure relate to methods of reducing the viscosity of a pretreated biomass mixture, by contacting a pretreated biomass mixture having an initial viscosity with CDH-heme domain polypeptides and GH61 polypeptides of the present disclosure; and incubating the contacted biomass mixture under conditions sufficient to reduce the initial viscosity of the pretreated biomass mixture. The present disclosure also provides methods of reducing the viscosity of a pretreated biomass mixture, by contacting a pretreated biomass mixture having an initial viscosity with CDH-heme domain polypeptides and GH61 polypeptides of the present disclosure and cellulases; and incubating the contacted biomass mixture under conditions sufficient to reduce the initial viscosity of the pretreated biomass mixture.

The disclosed methods may be carried out as part of a pretreatment process. The pretreatment process may include the additional step of adding CDH-heme domain polypeptides and GH61 polypeptides of the present disclosure and cellulases to pretreated biomass mixtures after a step of pretreating the biomass, and incubating the pretreated biomass with the CDH-heme domain polypeptides and GH61 polypeptides of the present disclosure and cellulases under conditions sufficient to reduce the viscosity of the mixture. The polypeptides or compositions may be added to pretreated biomass mixture while the temperature of the mixture is high, or after the temperature of the mixture has decreased. In some aspects, the methods are carried out in the same vessel or container where the pretreatment was performed. In other aspects, the methods are carried out in a separate vessel or container where the pretreatment was performed.

In some aspects, the methods are carried out in the presence of high salt, such as solutions containing saturating concentrations of salts, solutions containing sodium chloride (NaCl) at a concentration of at least at or about 0.1 M, 0.2 M, 0.3 M, 0.4 M, 0.5 M, 1 M, 1.5 M, 2 M, 2.5 M, 3 M, 3.5 M, or 4 M sodium chloride, or potassium chloride (KCl), at a concentration at or about 0.1 M, 0.2 M, 0.3 M, 0.4 M, 0.5 M, 1 M, 1.5 M, 2 M, 2.5 M 3.0 M or 3.2 M KCl and/or ionic liquids, such as 1,3-dimethylimidazolium dimethyl phosphate ([DMIM]DMP) or [EMIM]OAc, or in the presence of one or more detergents, such as ionic detergents (e.g., SDS, CHAPS), sulfydryl reagents, such as in saturating ammonium sulfate or ammonium sulfate between at or about 0 and 1 M. In other aspects, the methods are carried out over a broad temperature range, such as between at or about 20° C. and 50° C., 25° C. and 55° C., 30° C. and 60° C., or 60° C. and 110° C. In some aspects, the methods may be performed over a broad pH range, for example, at a pH of between about 4.5 and 8.75, at a pH of greater than 7 or at a pH of 8.5, or at a pH of at least 5.0, 5.5, 6.0, 6.5, 7.0, 7.5, 8.0, or 8.5.

Methods of Cleaving Cellulose Polymers into Specific Products

Further provided herein are methods for cleaving cellulose polymers into specific cleavage products. In one aspect, provided herein is a method for cleaving a cellulose polymer to yield a glucose molecule and a 4-keto glucose molecule. The glucose and 4-keto glucose molecules resulting from the cleavage of a cellulose polymer may remain as part of shorter cellulose polymers, being located at the ends of the shorter cellulose polymers that result from the cleavage of a longer cellulose polymer. In another aspect, provided herein is a method for cleaving a cellulose polymer to yield cellodextrins. In another aspect, provided herein is a method for cleaving a cellulose polymer to yield cellodextrins with the non-reducing sugar end containing a 4-keto glucose.

In a method for cleaving cellulose molecules into glucose and 4-keto glucose molecules, cellulose may be contacted by a GH61 polypeptide of the disclosure. In some aspects, in a method for cleaving cellulose molecules into glucose and 4-keto glucose molecules, cellulose is contacted by a GH61 polypeptide of the disclosure and a CDH-heme domain polypeptide of the disclosure. In another aspect, in a method for cleaving cellulose molecules into glucose and 4-keto glucose molecules, cellulose is contacted by a GH61 polypeptide of the disclosure, a CDH-heme domain polypeptide of the disclosure, and one or more cellulases. In another aspect, in a method for cleaving cellulose molecules into glucose and 4-keto glucose molecules, cellulose is contacted by a CDH-heme domain polypeptide of the present disclosure and a GH61 polypeptide having an amino acid sequence of GH61-1/NCU02240, GH61-2/NCU07898, GH61-4/NCU01050, GH61-5/NCU08760, NCU02916, or NCU00836. In another aspect, in a method for cleaving cellulose molecules into glucose and 4-keto glucose molecules, cellulose is contacted by a CDH-heme domain polypeptide of the present disclosure, a GH61 polypeptide having an amino acid sequence of GH61-1/NCU02240, GH61-2/NCU07898, GH61-4/NCU01050, GH61-5/NCU08760, NCU02916, or NCU00836, and one or more cellulases.

Methods of Cleaving Specific Bonds in Cellulose

Additionally provided herein are methods for cleaving specific bonds in cellulose polymers and related molecules. In one aspect, provided herein is a method for cleaving the 1-4 glycosidic bond that links glucose molecules in a cellulose polymer. In another aspect, provided herein is a method for cleaving the C—H bond on the 4 position of a glucose molecule, thereby facilitating the generation of a 4-keto glucose molecule.

In some aspects, in a method for cleaving the 1-4 glycosidic bond that links glucose molecules in a cellulose polymer, cellulose is contacted by a GH61 polypeptide of the disclosure. In another aspect, in a method for cleaving the 1-4 glycosidic bond that links glucose molecules in a cellulose polymer, cellulose is contacted by a GH61 polypeptide of the disclosure and a CDH-heme domain polypeptide of the disclosure. In another aspect, in a method for cleaving the 1-4 glycosidic bond that links glucose molecules in a cellulose polymer, cellulose is contacted by a GH61 polypeptide of the disclosure, a CDH-heme domain polypeptide of the disclosure, and one or more cellulases. In another aspect, in a method for cleaving the 1-4 glycosidic bond that links glucose molecules in a cellulose polymer, cellulose is contacted by a CDH-heme domain polypeptide of the present disclosure and a GH61 polypeptide having an amino acid sequence of GH61-1/NCU02240, GH61-2/NCU07898, GH61-4/NCU01050, GH61-5/NCU08760, NCU02916, or NCU00836.

In a method for cleaving the C—H bond on the 4 position of a glucose molecule, thereby facilitating the generation of a 4-keto glucose molecule, cellulose may be contacted by a GH61 polypeptide of the disclosure. In some aspects, in a method for cleaving the C—H bond on the 4 position of a glucose molecule, thereby facilitating the generation of a 4-keto glucose molecule, cellulose is contacted by a GH61 polypeptide of the disclosure and a CDH-heme domain polypeptide of the disclosure. In another aspect, in a method for cleaving the C—H bond on the 4 position of a glucose molecule, thereby facilitating the generation of a 4-keto glucose molecule, cellulose is contacted by a GH61 polypeptide of the disclosure, a CDH-heme domain polypeptide of the disclosure, and one or more cellulases. In another aspect, in a method for cleaving the C—H bond on the 4 position of a glucose molecule, thereby facilitating the generation of a 4-keto glucose molecule, cellulose is contacted by a CDH-heme domain polypeptide of the present disclosure and a GH61 polypeptide having an amino acid sequence of GH61-1/NCU02240, GH61-2/NCU07898, GH61-4/NCU01050, GH61-5/NCU08760, NCU02916, or NCU00836.

Methods of Producing GH61 Polypeptides Bound to Copper

Provided herein are methods of producing GH61 polypeptides that are bound to copper atoms. In one aspect, GH61 polypeptides that are bound to copper atoms are produced in cells that are grown in media that contain copper atoms. In another aspect, GH61 polypeptides that are bound to copper atoms are produced by incubating GH61 polypeptides in a solution that contains copper. GH61 polypeptides that are bound to copper atoms that may be produced include, without limitation, GH61-1/NCU02240, GH61-2/NCU07898, GH61-4/NCU01050, GH61-5/NCU08760, GH61-6/NCU02916, and GH61-3/NCU00836. GH61 polypeptides that are bound to copper atoms that may be produced also include, without limitation, polypeptides of the NCU02240/NCU01050 clade and GH61 polypeptides containing the motif H-X₍₄₋₈₎-Q-X-Y. GH61 polypeptides that are bound to copper atoms may be recombinant or naturally occurring.

Further provided herein are methods for producing compositions that contain multiple recombinant GH61 polypeptides, wherein 50% or more of the GH61 proteins are bound to a copper atom. Also provided herein are methods for producing compositions that contain multiple recombinant GH61 polypeptides, wherein 55%, 60%, 65%, 70%, 75%, 80%, 85%, 90%, 91%, 92%, 93%, 94%, 95%, 96%, 97%, 98%, 99%, or more, or 100% of the GH61 proteins are bound to a copper atom. GH61 polypeptides that are bound to copper atoms may be produced by any method wherein copper atoms are made available to GH61 polypeptides.

GH61 polypeptides that are bound to copper atoms may be produced in cells that are grown in media that contain copper atoms. Cells that are grown in media that contain copper atoms may be grown in media that contains at least 0.01, 0.05, 0.1, 0.5, 1, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, or 10,000 μM copper. Cells that are grown in media that contain copper atoms may be grown in media that contains no more than 0.05, 0.1, 0.5, 1, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, or 10,000 μM copper. In some aspects, cells that are grown in media that contain copper atoms may be grown in media that contains 0.1-1000 μM, 100-800 μM, 0.1-500 μM, or 1-50 μM copper.

Also provided herein are methods of producing GH61 polypeptides, wherein GH61 polypeptides are incubated in a solution that contains copper. GH61 polypeptides may be exposed to a metal chelating agent, such as EDTA or EGTA, prior to incubation in a solution that contains copper, in order to remove previously-bound metals from the GH61 polypeptide.

GH61 polypeptides that are incubated in a solution that contains copper may be incubated in a solution that contains at least 0.01, 0.05, 0.1, 0.5, 1, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, or 10,000 μM copper. GH61 polypeptides that are incubated in a solution that contains copper may be incubated in a solution that contains no more than 0.05, 0.1, 0.5, 1, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, or 10,000 μM copper. In some aspects, GH61 polypeptides that are incubated in a solution that contains copper may be incubated in a solution that contains 0.1-1000 μM, 100-800 μM, 0.1-500 μM, or 1-50 μM copper.

In the methods provided herein, copper may be added to a liquid by dissolving a copper salt in the liquid. Copper salts that may be used with the methods disclosed herein include any copper salt that dissolves in water, including without limitation, copper sulfate, copper acetate, copper carbonate, copper chloride, copper hydroxide, and copper nitrate.

Methods of Degrading Cellulose-Containing Materials with GH61 Polypeptides that are Bound to Copper

As used herein, “cellulose-containing materials” include any material that contains cellulose, including biomass. Provided herein is a method of degrading a cellulose-containing material wherein the method includes contacting the cellulose-containing material with a recombinant CDH-heme domain polypeptide and a recombinant GH61 polypeptide of the present disclosure, wherein the GH61 polypeptide is bound to a copper atom. Further provided herein is a method of degrading a cellulose-containing material, wherein the method includes contacting the cellulose-containing material with multiple recombinant CDH-heme domain polypeptides and multiple recombinant GH61 polypeptides of the disclosure, wherein 50%, 55%, 60%, 65%, 70%, 75%, 80%, 85%, 90%, 91%, 92%, 93%, 94%, 95%, 96%, 97%, 98%, 99%, or more, or 100% of the GH61 proteins are bound to a copper atom. Further provided herein is a method of degrading a cellulose-containing material, wherein the method includes contacting the cellulose-containing material with multiple recombinant CDH-heme domain polypeptides and multiple recombinant GH61 polypeptides of the present disclosure, wherein 55%, 60%, 65%, 70%, 75%, 80%, 85%, 90%, 91%, 92%, 93%, 94%, 95%, 96%, 97%, 98%, 99%, or more, or 100% of the GH61 proteins are bound to a copper atom and one or more of the GH61 polypeptides have the amino acid sequence of GH61-1/NCU02240, GH61-2/NCU07898, GH61-4/NCU01050, GH61-5/NCU08760, NCU02916, or NCU00836.

Also provided herein is a method of degrading a cellulose-containing material wherein the method includes contacting the cellulose-containing material with a recombinant CDH-heme domain polypeptide and a recombinant GH61 polypeptide of the present disclosure, and one or more cellulases, wherein the GH61 polypeptide is bound to a copper atom. Further provided herein is a method of degrading a cellulose-containing material, wherein the method includes contacting the cellulose-containing material with multiple recombinant CDH-heme domain polypeptides and multiple recombinant GH61 polypeptides of the disclosure, and one or more cellulases, wherein 50%, 55%, 60%, 65%, 70%, 75%, 80%, 85%, 90%, 91%, 92%, 93%, 94%, 95%, 96%, 97%, 98%, 99%, or more, or 100% of the GH61 proteins are bound to a copper atom. Further provided herein is a method of degrading a cellulose-containing material, wherein the method includes contacting the cellulose-containing material with multiple recombinant CDH-heme domain polypeptides and multiple recombinant GH61 polypeptides of the present disclosure, and one or more cellulases, wherein 55%, 60%, 65%, 70%, 75%, 80%, 85%, 90%, 91%, 92%, 93%, 94%, 95%, 96%, 97%, 98%, 99%, or more, or 100% of the GH61 proteins are bound to a copper atom and one or more of the GH61 polypeptides have the amino acid sequence of GH61-1/NCU02240, GH61-2/NCU07898, GH61-4/NCU01050, GH61-5/NCU08760, NCU02916, or NCU00836.

Also provided herein is a method of degrading a cellulose-containing material, wherein the method includes contacting the cellulose-containing material with a recombinant CDH-heme domain polypeptide and a recombinant GH61 polypeptide of the present disclosure, wherein copper atoms are present in the reaction mixture. In some reaction mixtures that contain a cellulose-containing material, a recombinant CDH-heme domain polypeptide and a recombinant GH61 polypeptide of the present disclosure, the concentration of copper is at least 0.01, 0.05, 0.1, 0.5, 1, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, or 10,000 μM. In some reaction mixtures that contain a cellulose-containing material, a recombinant CDH-heme domain polypeptide and a recombinant GH61 polypeptide of the present disclosure, the concentration of copper is no more than 0.05, 0.1, 0.5, 1, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, or 10,000 μM. In some reaction mixtures that contain a cellulose-containing material, a recombinant CDH-heme domain polypeptide and a recombinant GH61 polypeptide of the present disclosure, the concentration of copper is between 0.1-1000 μM, 100-800 μM, 0.1-500 μM, or 1-50 μM.

Also provided herein is a method of degrading a cellulose-containing material, wherein the method includes contacting the cellulose-containing material with a recombinant CDH-heme domain polypeptide and a recombinant GH61 polypeptide of the present disclosure, and one or more cellulases, wherein copper atoms are present in the reaction mixture. In some reaction mixtures that contain a cellulose-containing material, a recombinant CDH-heme domain polypeptide and a recombinant GH61 polypeptide of the present disclosure, and one or more cellulases, the concentration of copper is at least 0.01, 0.05, 0.1, 0.5, 1, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, or 10,000 μM. In some reaction mixtures that contain a cellulose-containing material, a recombinant CDH-heme domain polypeptide and a recombinant GH61 polypeptide of the present disclosure, and one or more cellulases, the concentration of copper is no more than 0.05, 0.1, 0.5, 1, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, or 10,000 μM. In some reaction mixtures that contain a cellulose-containing material, a recombinant CDH-heme domain polypeptide and a recombinant GH61 polypeptide of the present disclosure, and one or more cellulases, the concentration of copper is between 0.1-1000 μM, 100-800 μM, 0.1-500 μM, or 1-50 μM.

Methods of Analyzing the Copper Content of GH61 Polypeptides

Additionally provided herein are methods for analyzing the copper content of GH61 polypeptides. To determine the copper content of GH61 polypeptides in a composition containing multiple GH61 polypeptides, various techniques may be used. Generally, the techniques involve the steps of: 1) obtaining a sample of a composition containing GH61 polypeptides of interest; 2) determining the concentration of GH61 polypeptide in the composition; 3) determining the concentration of copper atoms in the composition, and 4) calculating the amount of copper atoms per GH61 polypeptide, based on the amount of GH61 polypeptides and copper atoms present in the sample.

The concentration of GH61 polypeptides in a sample may be determined through use of an assay for measuring protein content of a composition, such as a Bradford, Lowry, or bicinchoninic acid (BCA) assay. Given the mass of the protein content of a composition and the molecular weight of a GH61 polypeptide of interest, one of skill in the art can readily determine the concentration of GH61 polypeptides in a sample.

The concentration of copper atoms in a sample may be determined through use of any technique for the measurement of metal content of a composition, such as inductively coupled plasma atomic emission spectrometry or inductively coupled plasma mass spectrometry.

Given the concentration of GH61 polypeptides in a composition, and the concentration of copper atoms in the same composition, of one skill in the art can readily determine the percentage of GH61 polypeptides that are bound to a copper atom in a composition. Without being bound by theory, each GH61 polypeptide binds to one copper atom. For example, if the analysis of a composition containing purified GH61 polypeptides reveals that the composition contains about 80,000 GH61 polypeptides and 100,000 copper atoms per microliter of the sample, this indicates that 80% of the GH61 polypeptides in the sample are bound to a copper atom.

Method of Reducing the Amount of GH61 Polypeptides used for the Degradation of Cellulose-Containing Materials

Further provided herein are methods for reducing the amount of GH61 polypeptides used for the degradation of cellulose-containing materials. In some aspects, a method for reducing the amount of GH61 polypeptides used for the degradation of cellulose-containing materials involves providing multiple recombinant GH61 polypeptides, wherein 50%, 55%, 60%, 65%, 70%, 75%, 80%, 85%, 90%, 91%, 92%, 93%, 94%, 95%, 96%, 97%, 98%, 99%, or more, or 100% of the GH61 polypeptides are bound to a copper atom. In some aspects, a method for reducing the amount of GH61 polypeptides used for the degradation of cellulose-containing materials involves providing multiple recombinant GH61 polypeptides having the sequence of GH61-1/NCU02240, GH61-2/NCU07898, GH61-4/NCU01050, GH61-5/NCU08760, NCU02916, or NCU00836, wherein 50%, 55%, 60%, 65%, 70%, 75%, 80%, 85%, 90%, 91%, 92%, 93%, 94%, 95%, 96%, 97%, 98%, 99%, or more, or 100% of the GH61 polypeptides are bound to a copper atom. In some aspects, GH61 polypeptides that are bound to copper atoms are more effective at promoting the degradation of cellulose than GH61 polypeptides that are not bound to copper atoms. Accordingly, if GH61 polypeptides that are bound to copper atoms are used for the degradation of cellulose, less of these polypeptides may be needed to promote degradation of cellulose, as compared to GH61 polypeptides that are not bound to copper atoms.

Identification of CDH-Dependent Accessory Cellulase Systems

In another embodiment, disclosed herein are methods for identifying CDH-dependent accessory cellulase systems. As provided herein, accessory cellulase systems are compositions that increase the degradation of cellulose in reactions containing cellulose, cellulases, and other molecules. CDH-dependent accessory cellulase systems are compositions that typically require the presence of a CDH-heme domain polypeptide in order to increase the degradation of cellulose. In some aspects, a CDH-dependent accessory cellulase system is composed of one type of molecule. In some aspects, a CDH-dependent accessory cellulase system is composed of two or more types of molecule.

In one aspect, a method of identifying CDH-dependent cellulase systems includes the steps of: i) obtaining a sample of proteins secreted by a cellulase-secreting fungus (a “secretome”); ii) contacting a portion of the sample with EDTA or potassium cyanide; iii) measuring the cellulase activity of the EDTA or potassium cyanide-treated sample; iv) measuring the cellulase activity of the non-EDTA or potassium cyanide-treated sample; v) comparing the cellulase activity of the EDTA or potassium cyanide-treated sample with the cellulase activity of the non-EDTA or potassium cyanide-treated sample, in order to identify CDH-dependent accessory cellulase systems. Using this method, the identification of a significant difference in the extent of degradation of cellulose between an EDTA or potassium cyanide-treated sample and its corresponding non-treated sample suggests the presence of a CDH-dependent cellulase system in the sample. Different concentrations of EDTA or potassium cyanide may be used to assay for CDH-dependent accessory cellulase systems, including, without limitation, 0.001 mM, 0.01 mM, 0.1 mM, 1 mM, 10 mM, and 100 mM EDTA or potassium cyanide.

In one aspect, a method of identifying CDH-dependent cellulase systems includes the steps of: i) obtaining a sample of proteins secreted by a cellulase-secreting fungus (a “secretome”); ii) subjecting a portion of the sample to anaerobic conditions; iii) measuring the cellulase activity of the sample under anaerobic conditions; iv) measuring the cellulase activity of the sample that is not subjected to anaerobic conditions; v) comparing the cellulase activity of the sample subjected to anaerobic conditions with the cellulase activity of the sample that is not subjected to anaerobic conditions, in order to identify CDH-dependent accessory cellulase systems. Using this method, the identification of a significant difference in the extent of degradation of cellulose between the sample subjected to anaerobic conditions and its corresponding sample not subjected to ananerobic conditions suggests the presence of a CDH-dependent cellulase system in the sample.

Anaerobic conditions can be generated, for example, through use of an anaerobic chamber (such as from Coy Laboratory Products, Inc., Grass Lake, Mich.). In some aspects, a buffer may be sparged with a non-oxygen gas, such as nitrogen, to removed dissolved oxygen. In some aspects, a buffer may be stirred vigorously in an anaerobic chamber for an extended time period to remove dissolved oxygen.

### EXAMPLES

The following Examples are merely illustrative and are not meant to limit any aspects of the present disclosure in any way.

### Example 1

### Production of a Strain of N. crassa Containing a Deletion of NCU00206, Cdh-1

The Neurospora functional genomics project has generated knockout strains for most of the genes in the N. crassa genome using targeted gene replacement through homologous recombination. A heterokaryon strain of Δcdh-1 is available through the Fungal Genetic Stock Center (FGSC), but despite numerous attempts, a homokaryon strain could not be generated due to an ascospore-lethal linked mutation. To obtain a clean deletion of cdh-1, a N. crassa strain deficient in non-homologous end joining recombination was transformed with a cassette provided by the Neurospora functional genomics project. Heterokaryon transformants showing antibiotic resistance were genotyped using PCR to confirm the deletion of cdh-1. Transformants were crossed with wild-type N. crassa and 20 hygromycin resistant progeny were then screened for the production of CDH during growth on cellulose. The strains that showed the best growth on Avicel and that were also deficient in CDH activity in the culture filtrate were genotyped. Multiple homokaryon strains in which cdh-1 was deleted were confirmed by PCR.

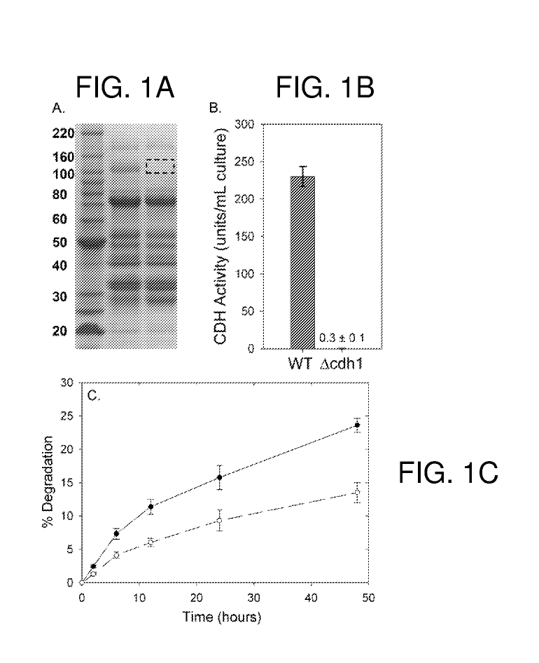

Growth of the Δcdh-1 strains in liquid culture on Vogel's salts supplemented with 2% sucrose was identical to that of wild-type. There was only a slight growth defect on Avicel, a pure form of crystalline cellulose. Both the wild-type and Δcdh-1 strains completely degraded all of the Avicel in the culture after 6-7 days of growth, as determined by light microscopy. The proteins present in the culture filtrate were analyzed by SDS-PAGE (FIG. 1A) and the extracellular proteins secreted by the Δcdh-1 strains were very similar to those of the wild-type, with the exception of the loss of the CDH-1 band between 100 and 120 kDa. The total secreted protein in the Δcdh-1 strains varied from ˜40% lower than the wild-type strain to equal to the wild-type strain for different transformants. CDH activity in the culture filtrate of the Δcdh-1 strains was on average 500 fold lower than in the wild-type culture filtrates (FIG. 1B).

Standard cellulase-specific activities of the Δcdh-1 strains and the wild-type were then compared. The endoglucanase activity and cellobiohydrolase activity, as measured by the azo-CMC and MULAC assays, respectively, were similar for the wild-type and Δcdh-1 strains when equal levels of total protein were loaded. Avicelase activity was 37-49% lower in the Δcdh-1 strain's culture filtrates than in the wild-type culture filtrates when loaded on an equal protein basis (FIG. 1C). Analysis of hydrolysis products after 24 hours of reaction time by HPLC showed that in the Δcdh-1 strain's culture filtrate glucose (>90%) was the main sugar produced, followed by cellobiose. In the wild-type culture filtrate, glucose remained the dominant product (80%), followed by cellobiose, cellobionic acid and trace amounts of gluconic acid. No additional peaks were present in the chromatograms.

Endoglucanase activity was determined by mixing appropriately diluted culture filtrate to the azo-CMC reagent (Megazyme SCMCL), according to the manufacturer's instructions. The rate of hydrolysis of 4-Methylumbelliferyl β-D-lactoside (MULAC) was determined by monitoring the increase in fluorescence (excitation λ=360 nm; emission λ=465 nm) upon addition of appropriately diluted culture filtrate to 1.0 mM MULAC.

### Example 2

### Stimulation of Cellulose Degradation by CDH

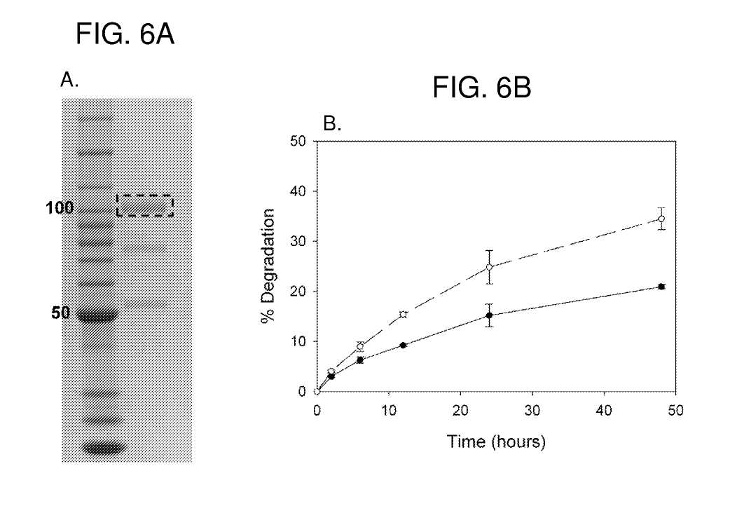

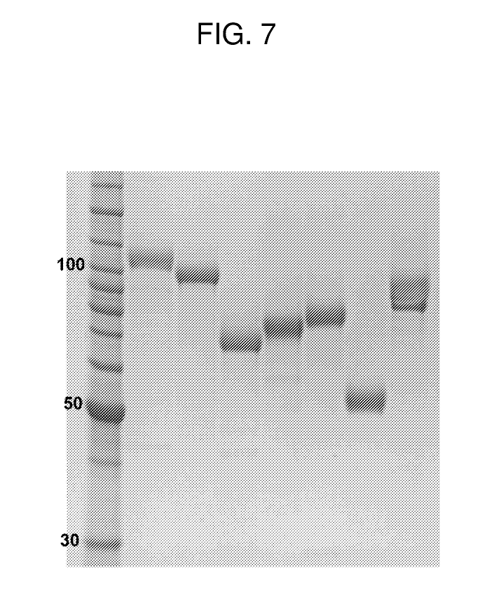

To more directly assess the contribution of CDH-1 to the degradation of cellulose, in vitro complementation assays were undertaken using purified CDHs. CDH-1 is difficult to isolate in pure form from N. crassa culture supernatants, and only a partially purified form of N. crassa CDH-1 could be isolated (FIG. 6A). The orthologous protein in the closely related thermophilic fungus, Myceliophthora thermophila, is easier to isolate in a pure form and was used for most of the complementation assays (FIG. 7). M. thermophila and N. crassa CDH-1 share 70% sequence identity and the same domain architecture. Both enzymes contain a C-terminal fungal cellulose binding domain. Individually, CDH-1 from M. thermophila had undetectable activity on Avicel, while the partially purified N. crassa CDH-1 had a slight hydrolytic activity due to low level contaminants.

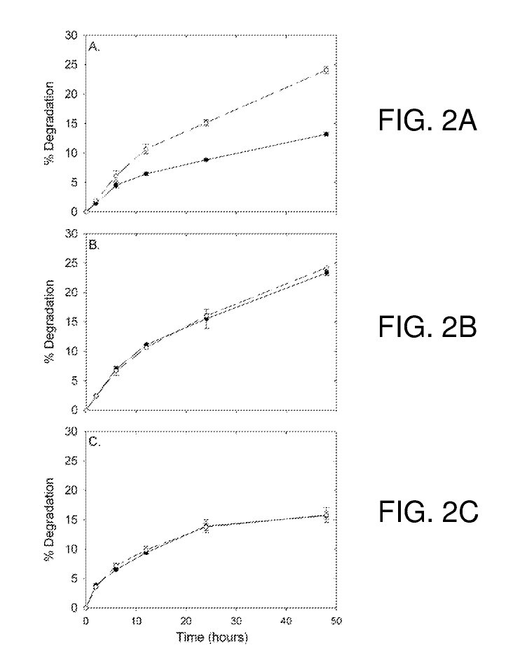

Addition of M. thermophila CDH-1 or partially purified N. crassa CDH-1 to the culture filtrate of the Δcdh-1 strains stimulated Avicel hydrolysis substantially (FIG. 2A and FIG. 6B). The Avicelase activity was 1.6-2.0 fold higher than the Δcdh-1 culture filtrate alone. Addition of CDH-1 to wild-type culture filtrate had no stimulatory effect on Avicel hydrolysis FIG. 2B). Further, CDH-1 was unable to stimulate a mixture of purified cellulases (FIG. 2C) from N. crassa including 2 cellobiohydrolases (CBH-1 and GH6-2), an endoglucanase (GH5-1), and a β-glucosidase (GH3-4) (FIG. 7).

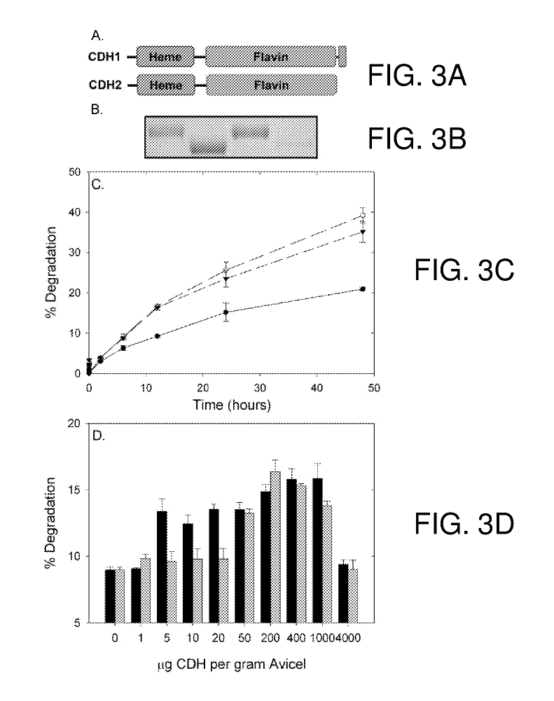

M. thermophila also produces a second CDH during growth on cellulose, CDH-2, which does not contain a fungal cellulose binding module (FIG. 3A). The cellulose binding propensity of M. thermophila CDH-1 and CDH-2 was analyzed using pull down experiments with Avicel (FIG. 3B). M. thermophila CDH-1 binds strongly to Avicel, while M. thermophila CDH-2 has only a very weak affinity. Aside from the different affinities for cellulose, M. thermophila CDH-1 and CDH-2 have very similar steady-state kinetic properties. At a CDH loading of 0.4 mg/g Avicel, CDH-2 was able to stimulate the hydrolysis of Avicel to the same extent as CDH-1 (FIG. 3C).

To further investigate the role of the cellulose binding module on the ability of CDH to stimulate Avicel hydrolysis, a titration experiment was performed (FIG. 3D). CDH-1 was able to stimulate the activity of the Δcdh-1 strain's culture filtrate at a 10 fold lower loading than CDH-2. A stimulatory effect on Avicelase activity in the Δcdh-1 culture filtrate was seen at a loading of 5 ug of CDH-1 per gram of Avicel while 50 ug of CDH-2 was required for a similar stimulation (FIG. 3D). At 4 mg CDH/g Avicel, both M. thermophila CDH-1 and CDH-2 have an inhibitory effect on Avicelase activity relative to the lower loadings.

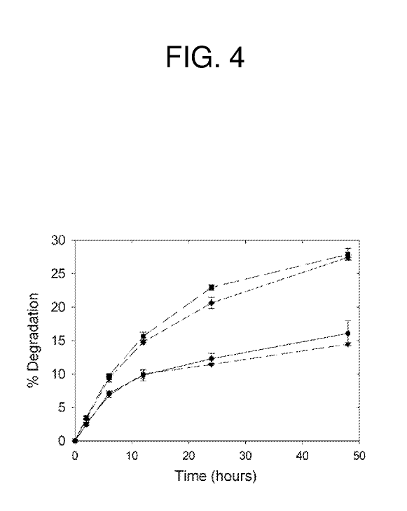

The flavin and heme domains of M. thermophila CDH-2 can be separated by cleavage with papain. To determine the contribution of the heme domain to the stimulation of activity we cleaved M. thermophila CDH-2 with papain and fractionated the flavin domain using size exclusion chromatography (FIG. 7). The flavin domain is able to oxidize cellobiose at the same rate as the full length enzyme when 2,6-dichlorophenolindophenol (DCPIP) is used as the electron acceptor, but has no activity when cytochrome C is used as the electron acceptor, reflecting on the importance of the heme domain for transfer to 1 electron acceptors. The flavin domain, when added on an equal activity basis as the full length CDH-2, is unable to stimulate the hydrolysis of Avicel by the Δcdh-1 strain's culture filtrate, despite production of cellobionic acid (FIG. 4). Even at a loading 10 fold higher than the full length CDH-2, the flavin domain is still unable to stimulate Avicel hydrolysis (data not shown), suggesting that the heme domain is essential for the stimulatory effect.

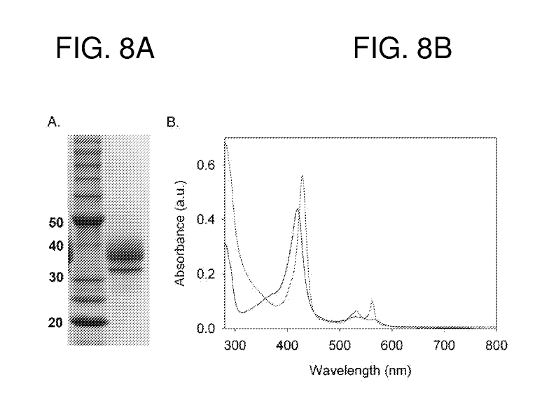

The heme domain of M. thermophila CDH-2 could not be sufficiently purified from the papain digestion of the full length protein and was thus recombinantly expressed in the yeast Pichia pastoris. The heme domain from CDH-2 was purified by nickel metal affinity chromatography and has the same spectral properties of the full length CDH-2 (FIG. 8). The recombinant heme domain was then tested for its ability to stimulate Avicel hydrolysis of the Δcdh-1 strain's culture filtrate (FIG. 4). Addition of the ferric heme domain at the same molar concentration as the full length CDH-2 required for maximum stimulation had no stimulatory effect. However, at a loading of 1 μM, the ferric heme domain was able to stimulate Avicelase activity to nearly the same extent as the full length enzyme at 23 nM (200 μg/g Avicel) (FIG. 4).

CDH activity assays were performed at room temperature by the addition of an appropriate amount of CDH or culture filtrate to a mixture containing 1.0 mM cellobiose, 200 uM DCPIP, and 100 mM sodium acetate pH 5.0. Reduction of DCPIP was monitored spectrophotometrically by the decrease in absorbance at 530 nm. One unit is equivalent to the number of micromoles of DCPIP reduced per minute.

All Avicelase assays were performed in triplicate with 10 mg/mL AVICEL™ PH101 (Sigma) in 50 mM sodium acetate pH 5.0 at 40° C. Assays were performed in 1.7 mL microcentrifuge tubes with 1.0 mL total volume and were inverted 20 times per minute. Each assay contained 0.05 mg/mL culture supernatant or 0.05 mg/mL reconstituted cellulase mixture containing CBH-1, GH6-2, GH5-1, and GH3-4 present in a ratio of 6:2.5:1:0.5. The concentration of heme domain used in stimulatory assays was 1.0 μM as determined by absorption at 430 nm of the fully reduced protein.

Assays were centrifuged for two minutes at 4000 rpm to pellet the remaining Avicel and 20 μL of assay mix was removed per well. Samples were incubated with 100 μL of desalted, diluted Novozymes 188 (Sigma) at 40° C. for 20 minutes to hydrolyze cellobiose and then 10-30 μL of the Novozymes 188 treated Avicelase assay supernatant was analyzed for glucose using the glucose oxidase/peroxidase assay as described previously (4). Percent degradation was calculated based on the amount of glucose measured relative to the maximum theoretical conversion of 10 mg/mL Avicel.

### Example 3

### Oxygen and Metal Ion Dependence on the Stimulation of Cellulose Degradation by CDH

The leading hypothesis for the biological function of CDH postulates that electrons from the heme domain of CDH are transferred to ferric complexes, quinones, molecular oxygen, or other redox mediators which lead to the production of radical species that can non-specifically degrade cellulose or lignin. We thus performed experiments to address if the stimulation of activity we had observed with CDH addition to the Δcdh-1 culture filtrate was due to a direct reaction with the cellulose or an indirect effect where metals or small molecules became reduced by CDH and subsequently contributed to the degradation.

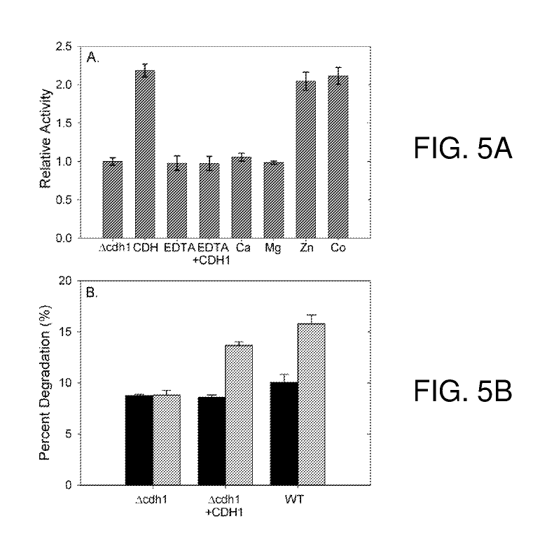

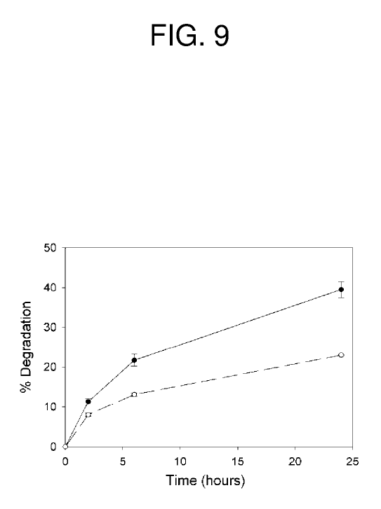

To test for the effect of small molecules in the Δcdh-1 culture we buffer exchanged the culture filtrate 10,000 fold using 10,000 MWCO spin concentrators. After buffer exchanging, CDH-1 was still able to stimulate the activity of the Δcdh-1 culture filtrate to the same extent. To test if there was a metal dependence for the stimulation, we incubated buffer exchanged culture filtrates from the Δcdh-1 cultures with 100 μM EDTA for 1 hour, and then performed an Avicelase assay. EDTA had no effect on the Avicelase activity of the Δcdh-1 culture filtrate; however, when M. thermophila CDH1 was added to the EDTA treated Δcdh-1 culture filtrate, no stimulatory effect was observed (FIG. 5A). Addition of EDTA to wild-type culture filtrate reduced Avicelase activity by ˜50% (FIG. 9). Taken together, these results suggest that there is a protein bound metal ion essential for the stimulation of cellulose degradation by CDH. Overnight incubation of M. thermophila CDH-1 with 1.0 mM EDTA had no effect on its ability to oxidize cellobiose with DCPIP or cytochrome C as electron acceptors (data not shown).

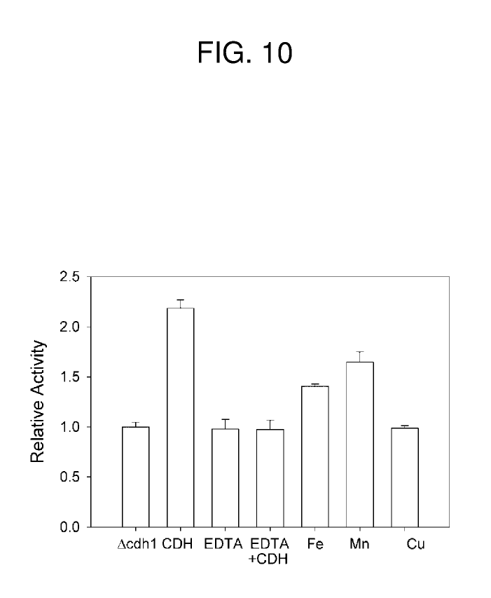

The identity of the metals responsible for the stimulation of Avicelase activity by CDH was next studied by the addition of various metal ions to buffer exchanged and EDTA treated Δcdh-1 culture filtrates at 1.0 mM concentrations (FIG. 5A). Addition of cobalt sulfate or zinc sulfate was able to fully rescue the stimulation of activity by CDH-1. Calcium chloride and magnesium sulfate, had no stimulatory effect. Redox-active metals known to inhibit cellulases (Feng et al. AEM 2010) including ferrous sulfate, manganese sulfate, and cuprous sulfate were also tested and while a stimulatory effect was initially observed (12 hours), inhibition by these metals was noted at longer timepoints (45 hours) (FIG. 10).

Finally, the role of molecular oxygen on the stimulation of activity by CDH-1 in the Δcdh-1 culture filtrate was explored. Avicelase activity of the Δcdh-1 culture filtrates is not affected by the presence of molecular oxygen, while in wild-type culture filtrates activity is reduced by ˜40% in the absence of oxygen. When purified M. thermophila CDH-1 was added to the Δcdh-1 culture filtrate under anaerobic conditions no stimulatory effect on Avicelase activity was observed, whereas stimulatory effect was observed under aerobic conditions (FIG. 5B).

Anaerobic Avicelase assays were performed as above except all assays were conducted in an anaerobic chamber (Coy) at room temperature. Buffers were sparged with nitrogen for 1 hour and culture filtrates were concentrated more than 20-fold to volumes of less than 300 μL before introduction into the anaerobic chamber. All solutions were left open in the anaerobic chamber for 72 hours before use to fully remove dissolved oxygen. Aerobic reactions were prepared in the anaerobic chamber in 3 mL reactivials and then removed from the anaerobic chamber, exposed to air, sealed, and returned to the anaerobic chamber. At specified timepoints, assays were centrifuged in the glove bag and 100 μL of assay mix was removed and analyzed by the glucose-oxidase peroxidase assay as described above.

### Example 4

### GH61 Proteins with Ability to Enhance Degradation of Cellulases in N. crassa

Proteomic analyses of N. crassa culture filtrate during growth on Avicel and Miscanthus led to the consistent identification of at least 4 GH61 proteins in the N. crassa secretome: GH61-4/NCU01050 (SEQ ID NO: 30), GH61-1/NCU02240 (SEQ ID NO: 24), GH61-2/NCU07898 (SEQ ID NO: 26), and GH61-5/NCU08760 (SEQ ID NO: 28).

### EDTA Treatment of Gene Deletions.

Addition of 1 mM EDTA to WT N. crassa culture filtrate inhibits cellulase activity roughly 2-fold presumably through removal of the surface exposed divalent metals that are required for GH61 catalytic activity. Addition of some divalent metals (Zn, Co, Mn, Fe, Cu) can restore cellulase activity after EDTA treatment. We determined that EDTA reduces the cellulase activity of the ΔNCU01050 and ΔNCU02240 knockouts by roughly 20-30%, and that EDTA reduces cellulase activity by about 50% in WT, ΔNCU07898 and ΔNCU08760 strains.

### Phylogenetic Analyses

Unlike N. crassa culture filtrate, the culture filtrate of M. thermophila during growth on Avicel is not inhibited by treatment with EDTA. A comparative analysis of the transcriptional responses both of these fungi have during growth on Avicel shows that while M. thermophila transcribes the genes orthologous to NCU08760 and NCU07898, it does not express genes orthologous to NCU01050 and NCU02240.

### Biochemical Fractionation

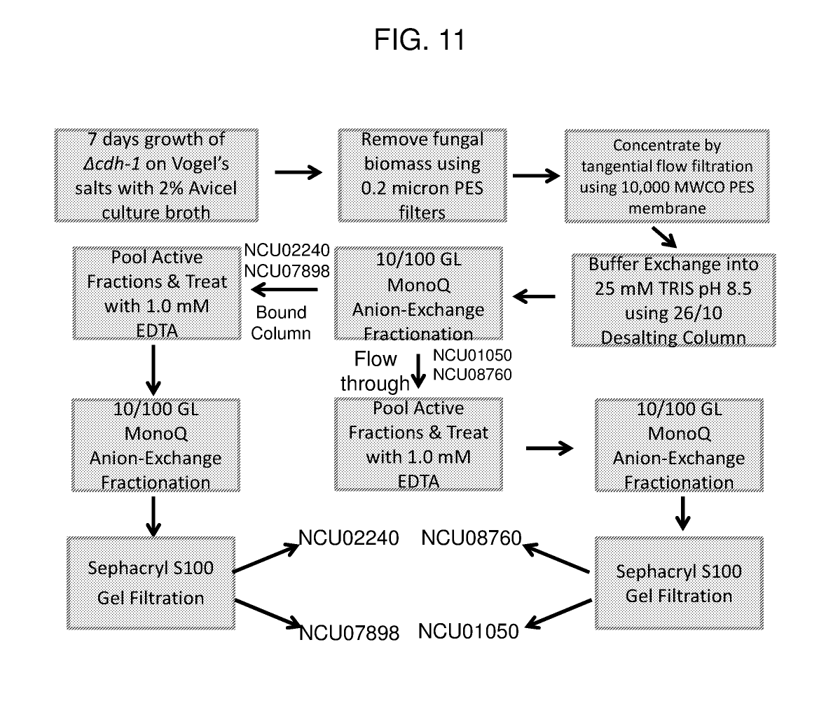

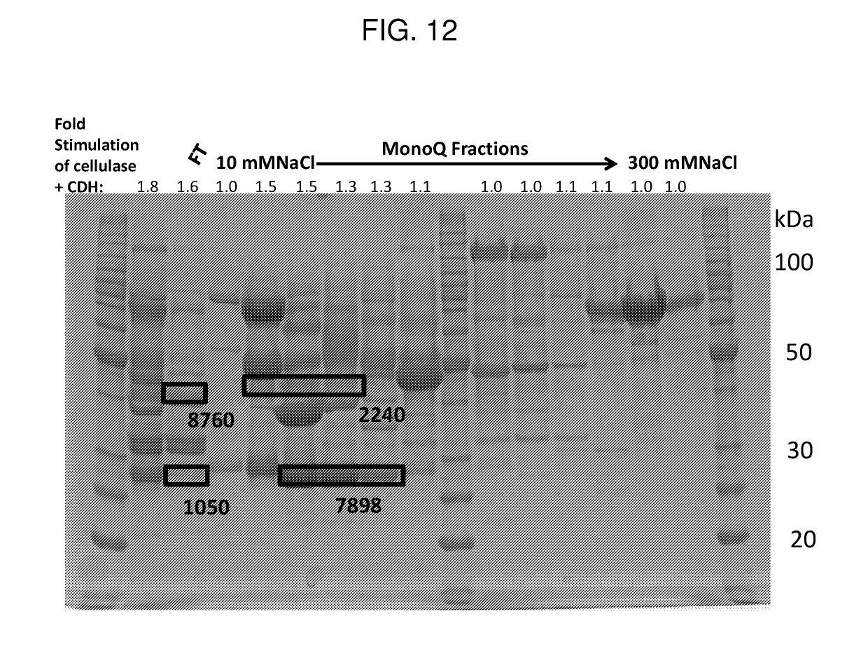

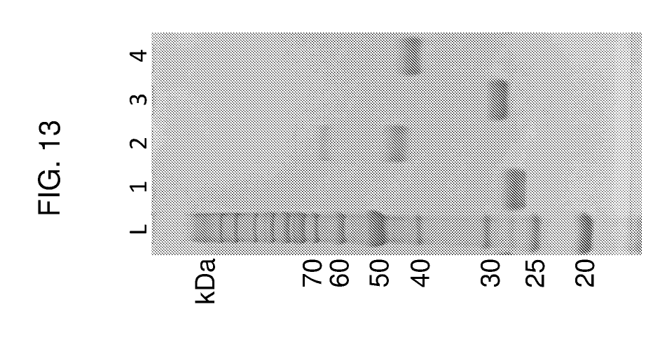

Δcdh-1 culture filtrate was concentrated, buffer exchanged, and separated using techniques of ion exchange and size exclusion chromatography. Fractions were assayed for their ability to show CDH dependent stimulation of basal cellulase activity. Fractions were further analyzed by SDS-PAGE and tryptic digests followed by liquid chromatography-tandem mass spectrometry (LC-MS/MS) to identify the proteins present in each fraction (FIGS. 11-13).

### Cellulase Assays

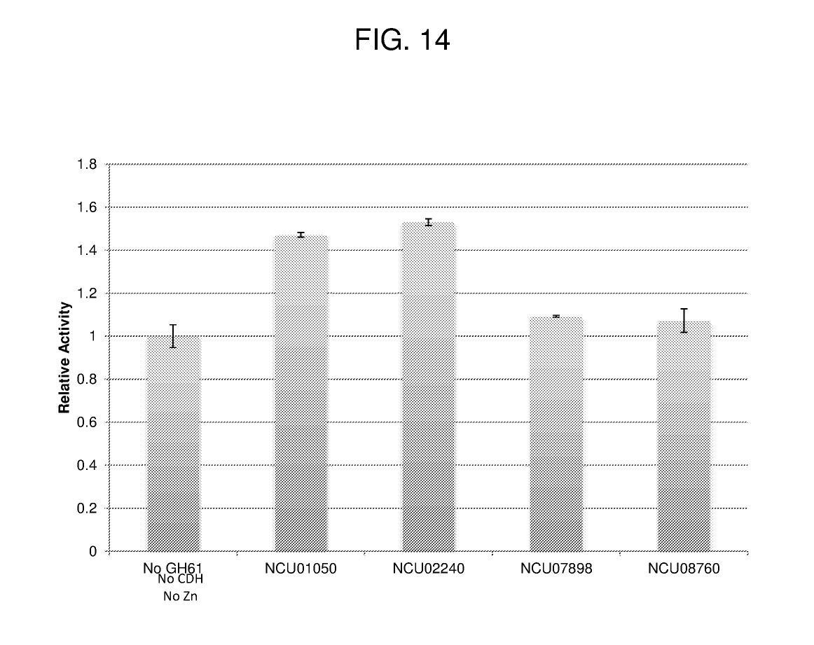

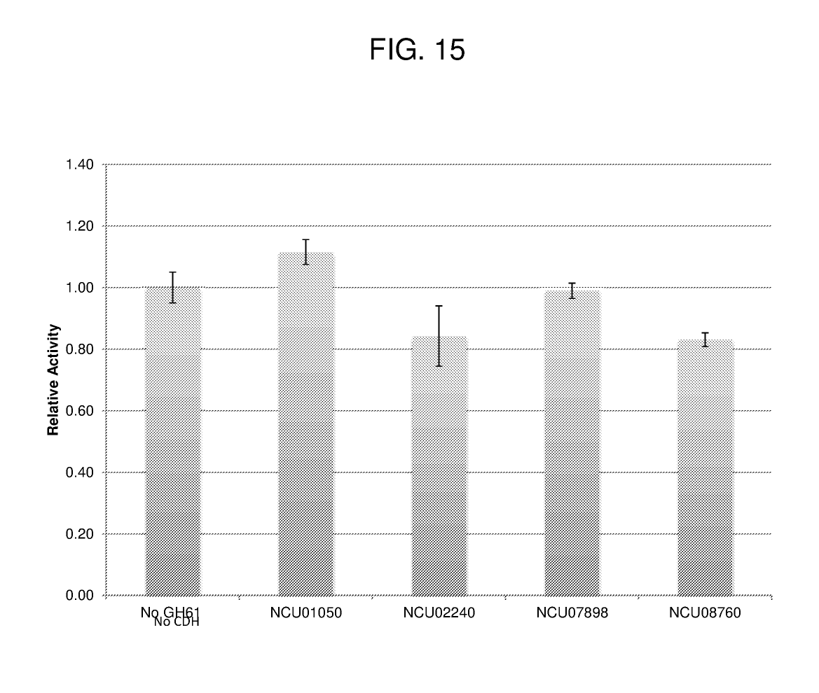

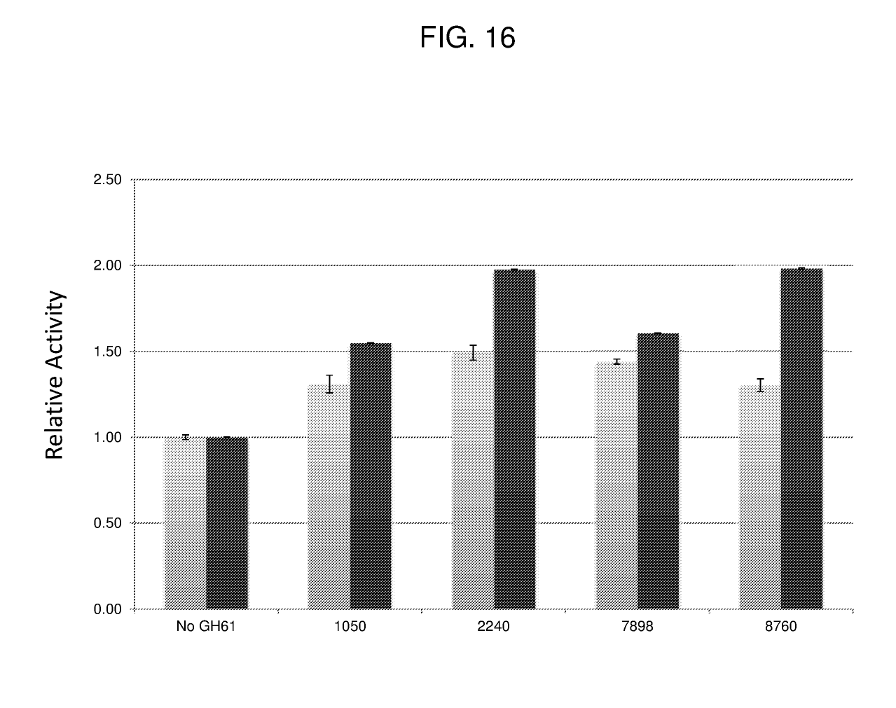

Cellulase assays with GH61 proteins, M. thermophila CDH-1, and cellulases were performed. In the experiments of FIG. 14, zinc-reconstituted N. crassa GH61 polypeptides were used with AVICEL™. In the experiments of FIG. 15, EDTA-treated N. crassa GH61 polypeptides were used with AVICEL™. In the experiments of FIG. 16, zinc-reconstituted N. crassa GH61 polypeptides were used with pretreated corn stover. NCU01050 and NCU02240 had the greatest effect at increasing degradation of AVICEL™, whereas NCU02240 and NCU08760 had the greatest effect at increasing degradation of pretreated corn stover.

### Example 5

### Mutational Analysis of GH61 Polypeptides

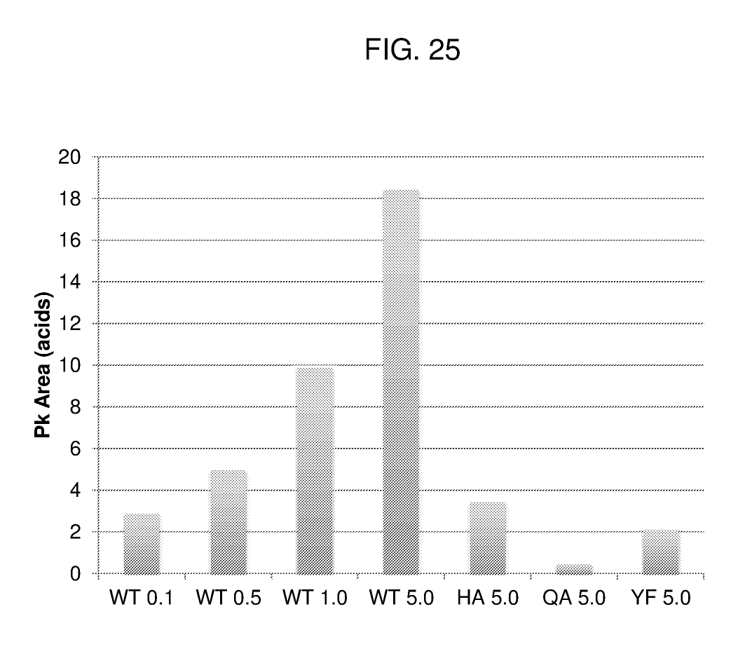

N. crassa NCU08760 [also known as N. crassa polysaccharide monooxygenase 1 (“PMO-1”)] polypeptides having a mutation in His-179, Gln-188, or Tyr-190 (numbering is based starting on the first amino acid of the signal peptide) were prepared and purified. Specifically, NCU08760 polypeptides having a H179A, Q188A, or Y190F mutation were prepared. These different mutant NCU08760 polypeptides were then assayed for activity on phosphoric acid swollen cellulose (“PASC”). FIG. 25 shows assay results comparing activity of each of the H179A (“HA”), Q188A (“QA”), or Y190F (“YF”) mutants with the activity of wild type (“WT”) NCU08760. The assay conditions were 5 mg/ml PASC, 2 mM ascorbic acid, and 50 mM sodium acetate, pH 5, and the assay was carried out at 40° C. with no mixing, and a 1-hour end point. As shown in FIG. 25, each of the HA, QA, and YF mutants had more than a 10-fold reduction in activity as compared with WT NCU08760, and the QA and YF mutants had more than a 50-fold reduction in activity as compared with WT NCU08760. Accordingly, these results indicate the importance of each of the amino acids of the H, Q, and Y amino acids of the H-X₍₄₋₈₎-Q-X-Y motif for GH61 activity.

1-18. (canceled)

19. A method of degrading cellulose, the method comprising contacting the cellulose with:
one or more cellulases,
a recombinant GH61 polypeptide; and
a recombinant CDH-heme domain polypeptide comprising a cellulose binding module (CBM),
wherein the contact occurs in a reaction mixture, and
wherein the contact occurs for a time sufficient to yield degraded cellulose.

20-27: (canceled)

28. The method of claim 19, wherein at least 50% of the GH61 polypeptides are bound to a copper atom.

29. The method of claim 19, wherein at least 90% of the GH61 polypeptides are bound to a copper atom.

30-31: (canceled)

32. The method of claim 19, wherein the recombinant GH61 polypeptide comprises the amino acid sequence of SEQ ID NO: 24, SEQ ID NO: 26, SEQ ID NO: 28, SEQ ID NO: 30, or SEQ ID NO: 90.

33-37: (canceled)

38. The method of claim 19, wherein the recombinant CDH-heme domain polypeptide comprises the amino acid sequence of SEQ ID NO: 32 or SEQ ID NO: 46.

39. The method of claim 19, wherein the CDH-heme domain comprises the amino acid sequence selected from the group consisting of SEQ ID NO: 70, SEQ ID NO: 76, SEQ ID NO: 80, and SEQ ID NO: 86, and wherein the CBM comprises the amino acid sequence of SEQ ID NO: 74 or SEQ ID NO: 84.

40. The method of claim 19, wherein the method further comprises having a concentration of between 0.1-500 μM copper in the reaction mixture.

41. The method of claim 40, wherein the concentration of copper in the reaction mixture is 1-50 μM.

42. The method of claim 19, wherein the recombinant GH61 polypeptide comprises an amino acid sequence having at least 80% sequence identity to the amino acid sequence of SEQ ID NO: 24, SEQ ID NO: 26, SEQ ID NO: 28, SEQ ID NO: 30, or SEQ ID NO: 90.

43. The method of claim 19, wherein the recombinant CDH-heme domain polypeptide comprises an amino acid sequence having at least 80% sequence identity to the amino acid sequence of SEQ ID NO: 32 or SEQ ID NO: 46.

44. The method of claim 19, wherein the CDH-heme domain comprises an amino acid sequence having at least 80% sequence identity to the amino acid sequence selected from the group consisting of SEQ ID NO: 70, SEQ ID NO: 76, SEQ ID NO: 80, and SEQ ID NO: 86, and wherein the CBM comprises an amino acid sequence having at least 80% sequence identity to the amino acid sequence of SEQ ID NO: 74 or SEQ ID NO: 84.

45. The method of claim 19, wherein the recombinant GH61 polypeptide comprises the motif H-X₍₄₋₈₎-Q-X-Y.

46. The method of claim 19, wherein the recombinant CDH-heme domain polypeptide comprises a first domain and a second domain, wherein the first domain comprises a CDH-heme domain and the second domain comprises a CBM, and wherein the polypeptide does not contain a dehydrogenase domain.

47. The method of claim 19, wherein the recombinant CDH-heme domain polypeptide comprises a first domain, a second domain, and a third domain, wherein the first domain comprises a CDH-heme domain, the second domain comprises a CBM, and the third domain comprises a dehydrogenase domain.


SEQUENCE LISTING (92 sequences)

SEQ ID NO: 1 - protein, 203 aa, Neurospora crassa
AESVAVHDAETGLTYSQNFALYKVDGRGITFRIAIPSNVSSNSAYDVVVQVIIPNDVGWAGLAWGGSMTKNPLMVFWRGSNNQPVLSSRSASHTPPQLYTTATYILFNTGTKSNSTHWQFTALCTGCTSWAADGGAVRYVQPNGGNRLAFAYSPTKPSNPSSPTSAITVHDVHAYWNHDFGTARNAGFEAAVQRLLGSQGVRA

SEQ ID NO: 2 - protein, 212 aa, Neurospora crassa
MSSASFLAEQQFEPDSSVYIDADTGLTFASYTSDRSIIFRVAIPDVIPADLIYDTVLQIVAPIDVGWAGFAWGGHMTYNPLGIAWTNDKEVVLSPRIAYGYYSPPIYTDSHYTVLKKGTHVNATHFQVTAKCTGCSSWGDDESTGISGNIDPEYQTTLAYAYGNTKVDTPADVQSTFGIHDSLGHPIYDLAVAKNKDFAEKVAALAAAGEAT

SEQ ID NO: 3 - protein, 196 aa, Neurospora crassa
KPVQSRDTVSAKYCDASTDICYSEFISPEKIAYRFAIPDNATAGNFDILLQIVAPKTVGWAGLAWGGVISWPYQSTIIVSSRKASARTYPQVSNDVSYKVLAGSGTNATHWTLNALAQGASAWGTTKLDPSSNAVPFAYAQSASPPTNPADAASRFSMHQSKGRWSHDLASGRIANFASAVEQLEKPEEEEKEEVK

SEQ ID NO: 4 - protein, 198 aa, Neurospora crassa
TDPVNKITLSTWRPDPGSNSGGGDAATYAFGLVLPPDALTKDANEYIGLLRCDVGDAASPGWCGVSHGQSGQMTQSLLLMAWASKGQVFTSFRYASGYNVPGLYTGNATLTQISATVNSTQFELIYRCQDCFAWNQGGSKGSVSTSSGLLVLGRAAAKGNLQNPTCPDKAIPGFHDNGFGQYGAPLEKVPHTSYSAWA

SEQ ID NO: 5 - protein, 195 aa, Podospora anserina
TDQTSGIKFKTWTQGTEATEASPFTFGLALPGDALTKNANEYLGILVRCKIEDAAAPGWCGLSHGQAGQMTNALLLVAWASEGTVYTSFRWATGYTLPGLYTGDAKLTQVSSNVTDTHFELIYRCQNCFSWNQDGTSGSVETTQGFLVLGHAAGSSGLENPTCPDRATFGFHDAGFGQWGAPLEGATSESYAEWA

SEQ ID NO: 6 - protein, 190 aa, Chaetomium globosum
TDEKTGITFNTWEATSGAAFTFGMALPADALTTDATEYIGLLRCAVADASAPGYCAISHGQSGQMSQALLLVAYASEGTVYTSFRYATGYTLPPLYTGDAKLTQISSTVSDTGFEVLFRCENCFAWDQDGATGSVSTTAGNLVLGRAAAKTGLEGASCPDTATFGFHDNGFGQWGAALEGAPSESYEEWA

SEQ ID NO: 7 - protein, 190 aa, Myceliophthora thermophila
TDEATGIQFKTWTASEGAPFTFGLTLPADALEKDATEYIGLLRCQITDPASPSWCGISHGQSGQMTQALLLVAWASEDTVYTSFRYATGYTLPGLYTGDAKLTQISSSVSEDSFEVLFRCENCFSWDQDGTKGNVSTSNGNLVLGRAAAKDGVTGPTCPDTAEFGFHDNGFGQWGAVLEGATSDSYEEWA

SEQ ID NO: 8 - protein, 192 aa, Myceliophthora thermophila
TDPDSGITFNTWGLAEDSPQTKGGFTFGVALPSDALTTDAKEFIGYLKCARNDESGWCGVSLGGPMTNSLLIAAWPHEDTVYTSLRFATGYAMPDVYQGDAEITQVSSSVNSTHFSLIFRCENCLQWSQSGATGGASTSNGVLVLGWVQAFADPGNPTCPDQITLEQHDNGMGIWGAQLNSDAASPSYTEWA

SEQ ID NO: 9 - protein, 193 aa, Neurospora crassa
THPDTGIVFNTWSASDSQTKGGFTVGMALPSNALTTDATEFIGYLECSSAKNGANSGWCGVSLRGAMTNNLLITAWPSDGEVYTNLMFATGYAMPKNYAGDAKITQIASSVNATHFTLVFRCQNCLSWDQDGVTGGISTSNKGAQLGWVQAFPSPGNPTCPTQITLSQHDNGMGQWGAAFDSNIANPSYTAWA

SEQ ID NO: 10 - protein, 187 aa, Podospora anserina
TDAETGIVFNSWGIPNGSPQSQGGWTFGMALPSDALSTDATEFIGYLDAAGWCGFSLAGPMTNSLLITAWPHEDTVYTTLRYAGGYAMPDKYAGNAEITQIRSSQNSTHFSLVFRCKNCLQWDHNGSTGGASTSGGFLVLGWVQAFPSPGNPTCPDQITLEQHDNGMGIWGAVLDENVANPSYTAWA

SEQ ID NO: 11 - protein, 197 aa, Aspergillus terreus
TDPDTGIVFDTWKIPAGTVTGGMTFGVALPSDALTTDATEFIGYLECALDASAGGWCGLSLGGSMTSNLLFMAYPYEDTVLTSLRFASGYVMPDVYAGNATVTQISSTVNSTHFTLLFRCEGCLSWNHNGQTGSASTSAGRLVLGWAQATESPTNPSCPDDISLVQHDSGSIWVATLDKNAASASYEEWTALANKTV

SEQ ID NO: 12 - protein, 192 aa, Aspergillus oryzae
TDTETGITFDTWSVPAGTGTGGLVFGVALPGSALTTDATEFIGYLQCASQNASSAGWCGISLGGGMNNNLLFLAYPYEDTVLTSLRFGSGYSMPGVYTGNANVTQISSSINATHFTLLFRCENCLTWDQNGQTGNATTSKGRLVLGWAQSTESPSNPSCPDNISLVQHDNQGIISATLDENAASASYEDWVK

SEQ ID NO: 13 - protein, 192 aa, Aspergillus nidulans
TDPDTGIVFDTWTVEASSSSAGFTFGVSLPEDALDTDATEFIGYLSCSSSSTSEFTGWCGLSMGSSMNSNLLLVAYAQDDTVLTSFRFSSGYAMPSVYSGNATLTQISSTVTADKFEVLFRCEECLRWDHEGVSGSATTSAGQLILAWAQAEESPTNADCPDDLSLVQHEAQGIWVGKLSGDAATSNYETWA

SEQ ID NO: 14 - protein, 185 aa, Phanerochaete chrysosporium
SASQFTDPTTGFQFTGITDPVHDVTYGFVFPPLATSGAQSTEFIGEVVAPIASKWIGIALGGAMNNDLLLVAWANGNQIVSSTRWATGYVQPTAYTGTATLTTLPETTINSTHWKWVFRCQGCTEWNNGGGIDVTSQGVLAWAFSNVAVDDPSDPQSTFSEHTDFGFFGIDYSTAHSANYQNYLN

SEQ ID NO: 15 - protein, 189 aa, Irpex lacteus
SASNYIDPDNGFQFTGVTDAETQVTYGVTFPPLATSGAQSTEFIGEVVAPVAAKWVGIALAGAMLQDLLLVAWPNAGKIVSSTRIASDYVQPTAYTGAATLTTLPETTVNATHWKWVFRCQGCTSWTSPSGSTGSISVDGSGVLAWAYSSVGVDDPTDPESTFQEHTSFGFFGIDYSQAHTSNYQNYLD

SEQ ID NO: 16 - protein, 180 aa, Grifola frondosa
SGSIYTDPGNGFTFDGITDPVYDVTYGVIFPTDTTSTEFIGEIVAPVAAQWIGVALGGAMIDNLLLVVWTNGNTIVSSTRYATDYIQPVPYAGPTLTTLPSSSVNSTHWKFVFRCQNCTSWLGGGSIPVSGSGVLAWAYSSIPVDDPADPNSDFLEHTDFGFFGMNFADAHTSNYNNYLN

SEQ ID NO: 17 - protein, 178 aa, Pycnoporus cinnabarinus
AAPYVDSGNGFVFDGITDPVYHVSYGIVLPQATTSSEFIGEIVAPLDAKWIGLALGGAMIGDLLIVAWPNGNEIVSSTRYATAYQLPDVYAGPTITTLPSSLVNSTHWKFVFRCQNCTSWEGGGGIDPTGTGVFAWAYSSVGVDDPSDPNTTFQEHTDFGFFGINFPDAQNSNYQNYL

SEQ ID NO: 18 - protein, 177 aa, Trametes versicolor
AAPYVDSGNGFVFDGVTDPVHSVTYGIVLPQASTSTEFIGEFVAPNEAQWIGLALGGAMIGNLLLVAWPNGNKIVSSPRYATGYTLPAAYAGPTITQLPSSSVNSTHWKFVFRCQNCTAWNGGSIDPSGTGVFAWAFSNVAVDDPSDPNSSFAEHTDFGFFGINFPDAQSSNYQNYL

SEQ ID NO: 19 - protein, 184 aa, Athelia rolfsii
SSYTDNGINFQGITDPTYGVTYGAVFPPASVDSDEFIGEIAAPVAAKWIGLSLGGAMINNLLIVAWPNNNEIVFSSRYTTGYVLPTIYSGPKITTISSSVNSTHWKWIYRCQNCTTWSGGSLAANGSAVWAWAYSSAAVDTPSSPSSSFDEHTDFGFFGEITSNAHVSQSVYEQYLTGTGVTST

SEQ ID NO: 20 - protein, 198 aa, Coprinopsis cinerea
QTESYVDPDTGITFQGRTDPVHGVTIGYVLPPLEPASDEFIGQILAPIENGWVGIAPGGGMINNLLVVAWPNGNEVVASVRMAKPFNDPVLTILPSTKVNATHWKLDYRCQGCTTWETANGPRSLPIDSAGAAAWALSKSPVDDPSDPDTTFAQHTDFGFYGQIWALSHVDAETYEHWASGGTGGGPTPTTPPTEPPT

SEQ ID NO: 21 - protein, 205 aa, Coprinopsis cinerea
QGSPTQWYDSITGVTFSRFYQQDTDASWGYIFPSASGGQAPDEFIGLFQGPASAGWIGNSLGGSMRNNPLLVGWVDGSTPRISARWATDYAPPSIYSGPRLTILGSSGTNGNIQRIVYRCQNCTRWTGGAGGIPTTGSAVFGWAFHSTTKPLTPSDPSSGLYRHSHAAQYGFDIGNARTTLYDYYLQQLTNAPPLSGGAPTQPPT

SEQ ID NO: 22 - protein, 203 aa, Coprinopsis cinerea
HGQVASQWYDSLTGVTWQRYYQQDFDASWGYLFPSSAGGAATDEFIGIFQAPANSGWIGNSLGGGMRNAPLIVGWVDGTTPRISARWATDYAPPSIYSGPRLTILGSSGSNGQIQRIVYRCQNCTSWSGGGIPSTGSSVLGWAFHATLQPLTPSDPNSGLYRHSAAGQHGFDLGTRTSSYNYFLQQLTNAPPLSGGAPTQPPT

SEQ ID NO: 23 - protein, 219 aa, Coprinopsis cinerea
MGDRAISTYAQDRPGTSEWCDSITDICFQRYYDADLDIAWGYVFPPSPSAGEPQPDEFIGLFTGPVSAGWIGNSLGGGMRSNPLVVGWVDNEHNALLSVRFTSRFASPDPLEGPQLTLLGTSGANATHQRIVYRCQNCTVWEGGSNGIRFNETAQFGFAAHGSQKPDDVANADSSVPVHSVAGQHDFDVSSARSDSYDMALQQLQAAPPLRPPIEEDAP

SEQ ID NO: 24 - protein, 322 aa, Neurospora crassa
MKVLSLLAAASAASAHTIFVQLEADGTTYPVSYGIRTPSYDGPITDVTSNDLACNGGPNPTTPSDKIITVNAGSTVKAIWRHTLTSGADDVMDASHKGPTLAYLKKVDDALTDTGIGGGWFKIQEDGYNNGQWGTSTVITNGGFQYIDIPACIPSGQYLLRAEMIALHAASSTAGAQLYMECAQINIVGGTGGTALPSTTYSIPGIYKATDPGLLVNIYSMSPSSTYTIPGPAKFTCPAGNGGGAGGGGSTTTAKPASSTTSKAAITSAVTTLKTSVVAPQPTGGCTAAQWAQCGGMGFSGCTTCASPYTCKKMNDYYSQCS

SEQ ID NO: 25 - DNA, 969 bp, Neurospora crassa
atgaaggtcctctccctcctcgccgccgcctctgcggcctcagcccacaccatcttcgtccagctcgaagccgacggcaccacctacccggtctcctacggaatccggaccccatcctacgatggtcccatcaccgacgtgacctccaacgaccttgcttgcaacggcggccccaaccccaccactccctctgacaagatcatcaccgtcaacgccggcagcaccgttaaggccatctggagacacactctcacttccggcgccgacgatgtcatggacgccagccacaagggccctacccttgcctacctcaagaaggtcgacgacgccttgactgacactggtatcggcggtggatggttcaagattcaagaagacggctacaacaacggccaatggggtaccagcaccgtcatcaccaacggtggtttccagtacatcgacatccccgcctgcatcccctcaggccaatacctcctccgcgccgagatgatcgccctgcacgccgcctcctccaccgccggcgcccaactctacatggaatgcgcccaaatcaacatcgtcggcggcaccggcggcaccgctctcccctccaccacctactcgatccccggcatctacaaggccactgaccccggtctgttggtcaacatctactccatgagcccaagcagcacttataccattcctggcccggccaagtttacttgcccggctggaaacggtggtggtgctggtggtggtggttctaccactactgctaagccggctagtagcaccaccagcaaggcggcgattaccagcgcggtcacaacgttgaagacgagcgtcgttgctcctcagcctactggtggttgcacggctgcgcagtgggcgcagtgcggtgggatgggattctcggggtgcactacttgtgcgagcccgtatacttgcaagaagatgaatgattattattcgcagtgctcgtaa

SEQ ID NO: 26 - protein, 241 aa, Neurospora crassa
MKTFATLLASIGLVAAHGFVDNATIGGQFYQFYQPYQDPYMGSPPDRISRKIPGNGPVEDVTSLAIQCNADSAPAKLHASAAAGSTVTLRWTIWPDSHVGPVITYMARCPDTGCQDWTPSASDKVWFKIKEGGREGTSNVWAATPLMTAPANYEYAIPSCLKPGYYLVRHEIIALHSAYSYPGAQFYPGCHQLQVTGSGTKTPSSGLVSFPGAYKSTDPGVTYDAYQAATYTIPGPAVFTC

SEQ ID NO: 27 - DNA, 726 bp, Neurospora crassa
atgaagacctttgcgactcttttggcttccatcggcctggtggccgctcacggctttgttgataacgccactattggtggtcagttttatcaattctaccagccgtaccaggacccctacatgggcagcccccccgatcgaatctctcgtaagattcccggcaacggccccgtcgaagacgtcacttccctcgccattcagtgcaacgccgactcagccccggccaagcttcatgcgtccgccgccgccggatcgactgtcactttgcgctggaccatttggcccgactcgcacgtgggacccgtcatcacctacatggcccgctgtcccgacacggggtgccaggactggacccctagcgccagtgataaggtgtggttcaagattaaggaaggtgggagggagggaacgagtaatgtttgggctgctacccccctcatgaccgccccggccaactacgagtacgccatcccgtcctgcctcaagcccggttactatctggttaggcacgagatcattgcgctgcacagcgcctactcttatcctggtgctcagttctacccgggatgccatcagttgcaggtgacaggttcgggaaccaagacgcccagctcgggactggtcagtttcccgggcgcgtacaagagtactgatccgggggttacttatgatgcttaccaggctgccacttataccatccccggtcctgctgtgtttacttgctaa

SEQ ID NO: 28 - protein, 342 aa, Neurospora crassa
MRSTLVTGLIAGLLSQQAAAHATFQALWVDGADYGSQCARVPPSNSPVTDVTSNAMRCNTGTSPVAKKCPVKAGSTVTVEMHQSHPPVPTLTYKQQANDRSCSSEAIGGAHYGPVLVYMSKVSDAASADGSSGWFKIFEDTWAKKPSSSSGDDDFWGVKDLNSCCGKMQVKIPSDIPAGDYLLRAEVIALHTAASAGGAQLYMTCYQISVTGGGSATPATVSFPGAYKSSDPGILVDIHSAMSTYVAPGPAVYSGGSSKKAGSGCVGCESTCKVGSGPTGTASAVPVASTSAAAGGGGGGGSGGCSVAKYQQCGGTGYTGCTSCASGSTCSAVSPPYYSQCV

SEQ ID NO: 29 - DNA, 1029 bp, Neurospora crassa
atgcggtccactcttgtcaccggcctcatcgccggcctactctcccaacaagccgccgcccacgccaccttccaagccctttgggtcgatggtgccgattatggctcgcaatgcgctcgcgtccctccttccaactcccccgtcaccgatgtgactagcaatgccatgaggtgtaacacgggaacttcgcccgttgcgaagaagtgccctgtcaaggcgggaagtacggtcactgttgagatgcaccagtcacaccctcccgtaccgacgctgacctataagcagcaagcaaatgaccgctcctgttcctctgaagccatcggtggcgctcactacggtcccgtcctcgtgtatatgtccaaggtctccgacgccgcctccgccgacggttcctctggctggttcaagatctttgaggacacctgggccaagaagccctccagctcctcgggcgacgatgatttctggggcgtcaaagacctcaactcgtgctgcggcaagatgcaggtcaagatcccctcggacatccccgcgggtgactatctcctccgtgccgaggttatcgcgctccataccgccgcaagcgcgggaggtgcccagttgtacatgacctgctaccagatctccgttaccggtggtggctccgctaccccggcgactgtcagctttcctggtgcctacaagagctccgaccctggtatcctcgttgacatccacagtgccatgagcacctacgtcgcccccggaccggctgtgtactcgggtggaagctccaagaaggccggaagcggctgcgtgggctgcgagtctacttgcaaggttggctccggcccgactggaactgcttctgccgtccctgttgcgagcacgtcggcggctgctggtggtggaggcggtggtgggagcggtggctgcagcgttgcaaagtatcagcagtgtggtggaaccggctataccgggtgcacatcctgcgcttccggatccacctgcagcgctgtctcacctccttattactcccagtgtgtctaa

SEQ ID NO: 30 - protein, 238 aa, Neurospora crassa
MKVLAPLVLASAASAHTIFSSLEVNGVNQGLGEGVRVPTYNGPIEDVTSASIACNGSPNTVASTSKVITVQAGTNVTAIWRYMLSTTGDSPADVMDSSHKGPTIAYLKKVDNAATASGVGNGWFKIQQDGMDSSGVWGTERVINGKGRHSIKIPECIAPGQYLLRAEMIALHAASNYPGAQFYMECAQLNVVGGTGAKTPSTVSFPGAYSGSDPGVKISIYWPPVTSYTVPGPSVFTC

SEQ ID NO: 31 - DNA, 717 bp, Neurospora crassa
atgaaggtcctcgcccctctcgtactcgcaagcgcagccagcgctcacaccattttctcctccctcgaggtcaacggcgtcaaccaaggcttgggagagggcgtccgcgtgcccacctacaacggtcccattgaggacgtcacctcggcctccatcgcctgcaacggctcgcccaacaccgtcgcctccacctccaaggtgatcaccgtgcaggcgggcaccaacgtgacggccatctggcgctacatgctcagcaccacgggcgactcgccggcggacgtcatggacagctcgcacaagggtcccaccatcgcctacctcaaaaaggttgacaacgccgccaccgccagcggtgtggggaatggctggttcaagatccagcaggacggcatggacagcagcggcgtctggggcaccgagcgcgttatcaacggcaagggccgccacagcatcaagatccccgagtgcatcgctccaggacagtacttactcagggctgagatgattgcgctgcacgcggcgagcaactatcctggtgcgcaattctacatggagtgtgcgcagcttaatgtcgttggtggtacgggtgctaagaccccttcgactgtcagctttcctggggcttactcgggctctgaccccggagtcaagattagcatctactggcctccggttacgtcttataccgtccctggtcccagtgtgtttacttgctaa

SEQ ID NO: 32 - protein, 829 aa, Neurospora crassa
MRTTSAFLSGLAAVASLLSPAFAQTAPKTFTHPDTGIVFNTWSASDSQTKGGFTVGMALPSNALTTDATEFIGYLECSSAKNGANSGWCGVSLRGAMTNNLLITAWPSDGEVYTNLMFATGYAMPKNYAGDAKITQIASSVNATHFTLVFRCQNCLSWDQDGVTGGISTSNKGAQLGWVQAFPSPGNPTCPTQITLSQHDNGMGQWGAAFDSNIANPSYTAWAAKATKTVTGTCSGPVTTSIAATPVPTGVSFDYIVVGGGAGGIPVADKLSESGKSVLLIEKGFASTGEHGGTLKPEWLNNTSLTRFDVPGLCNQIWKDSDGIACSDTDQMAGCVLGGGTAINAGLWYKPYTKDWDYLFPSGWKGSDIAGATSRALSRIPGTTTPSQDGKRYLQQGFEVLANGLKASGWKEVDSLKDSEQKNRTFSHTSYMYINGERGGPLATYLVSAKKRSNFKLWLNTAVKRVIREGGHITGVEVEAFRNGGYSGIIPVTNTTGRVVLSAGTFGSAKILLRSGIGPKDQLEVVKASADGPTMVSNSSWIDLPVGHNLVDHTNTDTVIQHNNVTFYDFYKAWDNPNTTDMNLYLNGRSGIFAQAAPNIGPLFWEEITGADGIVRQLHWTARVEGSFETPDGYAMTMSQYLGRGATSRGRMTLSPTLNTVVSDLPYLKDPNDKAAVVQGIVNLQKALANVKGLTWAYPSANQTAADFVDKQPVTYQSRRSNHWMGTNKMGTDDGRSGGTAVVDTNTRVYGTDNLYVVDASIFPGVPTTNPTAYIVVAAEHAAAKILAQPANEAVPKWGWCGGPTYTGSQTCQAPYKCEKQNDWYWQCV

SEQ ID NO: 33 - DNA, 2490 bp, Neurospora crassa
atgaggaccacctcggcctttctcagcggcctggcggcggtggcttcattgctgtcgcccgccttcgcccaaaccgctcccaagaccttcactcatcctgataccggcattgtcttcaacacatggagtgcttccgattcccagaccaaaggtggcttcactgttggtatggctctgccgtcaaatgctcttactaccgacgcgactgaattcatcggttatctggaatgctcctccgccaagaatggtgccaatagcggttggtgcggtgtttctctcagaggcgccatgaccaacaatctactcattaccgcctggccttctgacggagaagtctacaccaatctcatgttcgccacgggttacgccatgcccaagaactacgctggtgacgccaagatcacccagatcgcgtccagcgtgaacgctacccacttcacccttgtctttaggtgccagaactgtttgtcatgggaccaagacggtgtcaccggcggcatttctaccagcaataagggggcccagctcggttgggtccaggcgttcccctctcccggcaacccgacttgccctacccagatcactctcagtcagcatgacaacggtatgggccagtggggagctgcctttgacagcaacattgccaatccctcttatactgcatgggctgccaaggccaccaagaccgttaccggtacttgcagtggtccagtcacgaccagtattgccgccactcctgttcccactggcgtttcttttgactacattgtcgttggtggtggtgccggtggtattcccgtcgctgacaagctcagcgagtccggtaagagcgtgctgctcatcgagaagggtttcgcttccactggtgagcatggtggtactctgaagcccgagtggctgaataatacatcccttactcgcttcgatgttcccggtctttgcaaccagatctggaaagactcggatggcattgcctgctccgataccgatcagatggccggctgcgtgctcggcggtggtaccgccatcaacgccggtctctggtacaagccctacaccaaggactgggactacctcttcccctctggctggaagggcagcgatatcgccggtgctaccagcagagccctctcccgcattccgggtaccaccactccttctcaggatggaaagcgctaccttcagcagggtttcgaggttcttgccaacggcctcaaggcgagcggctggaaggaggtcgattccctcaaggacagcgagcagaagaaccgcactttctcccacacctcatacatgtacatcaatggcgagcgtggcggtcctctagcgacttacctcgtcagcgccaagaagcgcagcaacttcaagctgtggctcaacaccgctgtcaagcgcgtcatccgtgagggcggccacattaccggtgtggaggttgaggccttccgcaacggcggctactccggaatcatccccgtcaccaacaccaccggccgcgtcgttctttccgccggcaccttcggcagcgccaagatccttctccgttccggcattggccccaaggaccagctcgaggtggtcaaggcctccgccgacggccctaccatggtcagcaactcgtcctggattgacctccccgtcggccacaacctggttgaccacaccaacaccgacaccgtcatccagcacaacaacgtgaccttctacgacttttacaaggcttgggacaaccccaacacgaccgacatgaacctgtacctcaatgggcgctccggcatcttcgcccaggccgcgcccaacattggccccttgttctgggaggagatcacgggcgccgacggcatcgtccgtcagctgcactggaccgcccgcgtcgagggcagcttcgagacccccgacggctacgccatgaccatgagccagtaccttggccgtggcgccacctcgcgcggccgcatgaccctcagccctaccctcaacaccgtcgtgtctgacctcccgtacctcaaggaccccaacgacaaggccgctgtcgttcagggtatcgtcaacctccagaaggctctcgccaacgtcaagggtctcacctgggcttaccctagcgccaaccagacggctgctgattttgttgacaagcaacccgtaacctaccaatcccgccgctccaaccactggatgggcaccaacaagatgggcaccgacgacggccgcagcggcggcaccgcagtcgtcgacaccaacacgcgcgtctatggcaccgacaacctgtacgtggtggacgcctcgattttccccggtgtgccgaccaccaaccctaccgcctacattgtcgtcgccgctgagcatgccgcggccaaaatcctggcgcaacccgccaacgaggccgttcccaagtggggctggtgcggcgggccgacgtatactggcagccagacgtgccaggcgccatataagtgcgagaagcagaatgattggtattggcagtgtgtgtag

SEQ ID NO: 34 - protein, 4 aa, Artificial Sequence
  Sequence Motif
HTIF

SEQ ID NO: 35 - protein, 8 aa, Artificial Sequence
  Sequence Motif
RXPXYXGP

SEQ ID NO: 36 - protein, 8 aa, Artificial Sequence
  Sequence Motif
CNGXPNXX

SEQ ID NO: 37 - protein, 18 aa, Artificial Sequence
  Sequence Motif
DXXDXXHKGPXXAYXKKV

SEQ ID NO: 38 - protein, 6 aa, Artificial Sequence
  Sequence Motif
GWXKIX

SEQ ID NO: 39 - protein, 42 aa, Artificial Sequence
  Sequence Motif
IPXCIXXGQYLLRXEXXALHXAXXXXGAQXYMECAQXNXVGG

SEQ ID NO: 40 - protein, 20 aa, Artificial Sequence
  Sequence Motif
TXSXPGXYXXXDPGXXXXXY

SEQ ID NO: 41 - DNA, 2918 bp, Neurospora crassa
atgaaggtcttcacccgcattggaacgatcgttctggcgacgtcactgtgtaagttgttcttcggtacctcccatcggtggcccttcgcatcgtctgataccagtcaccctcaacagacctacagcaatgctccgctcaatacatcaacgagcaatataccgatcccgtgaacaagatcaccctcagcacctggcggccagaccctggttctaattctgggggtggagatgctgccacctacgcctttggcttggtcttgcctccggatgctctgaccaaagatgccaacgaatacatcggtctcttggtacggcgccctccgccacttccttgctctagggtggacatcagctgacacgattggtagcgctgtgatgttggtgatgcggcgagccccggatggtgtggtgtctcccacggccagtctggacaaatgacacagtcgttgttgctcatggcttgggcctccaagggtcaagtctttacctcatttcgctacgcatccggttataatgtgccaggactctacaccggaaatgcaaccctgacccagatctctgccactgtgaactcgacacagttcgaattgatctatcgctgccaggactgttttgcatggaaccaaggaggaagcaagggaagcgtatcaaccagcagtggccttctcgtcttgggccgtgccgcggccaagggaaatcttcagaacccgacttgccctgacaaggccattcccggctttcatgacaatgggtttggtcaatatggagcgcctctcgagaaagtcccgcatacctcatactcagcttgggcttctttagccacgaagaccactactgctgactgctctgggtacgttttgttctatgcgctttgttcacatatggttactaacatgtgctgaaacagggcatccgacccagtacccactggatccgagccgccagccgagccaacttcgacagcggagcccgttcccgtttgcacacctgccccaagcaagacgtacgactacatcatcgttggcgccggtgctggtggcattcccattgcggacaagctcagcgaggccggaaaaagtgtgttgttgatcgaaaagggacctccctccactggaagatggaagggcaccatgaagcctgagtggcttcagggcacgaacttgactcgcttcgatgttcctggtctatgcaaccagatctgggtggactctgccggcatcgcctgtacagataccgaccaaatggcgggatgtgtcctgggcggaggaacggctgttaatgccggcctgtggtggaaggtaagttgctttagttctattgatcaggaaagtcgcccactaaccgcgaaccatagccgcatcctcaggattggaactacaacttccccgagggctggaagtcgagagataccgtgccagccactaaccgtgtgttcggtcgcattcctggaacttggcatccttcgcaaaacggcaagctgtaccgacaagagggcttcaacgtcctagccagcgggctgagcaagagcggttggaaggaggtgatccccaacgatgcatacaaccagaagaaccacacctttggtcacagcaccttcatgttcgctaaaggcgagcgaggtggccctctggcaacataccttgtgacggcggtagctcgcaagcagttcactctctggaccaatgtagctgtgagaagggcagttcgtaacggaagccgtatcactggcgttgagctcgaatgcttgacggatggtggtctcagcggaactgtcaacgtgacccctaacactggccgtgttatctttgctgcaggcacttttggttccgccaagcttctccttcgcagtaagttatcatgttgatgtgtgatgttacattggatgacttgtccgctgacaggtacgacacaggcggtatcggacctaccgatcaactcgagattgtcaaggggtcgacggatggcccaacgttcatttccaaggaccaatggatcaaccttccagttggctacaacctcatggatcatctcaacactgatctcattatcacccatcctgacgttgtcttctacgacttctacgaggcttggaacacgcccattgaaggtgacaagagcgcctatcttcagaatagatctggaatccttgcccaggctgctcccaatattggtcctttggtacgtggcatcaggtgtagtacggtcgatcgagtctggctaacatgtgactctacagatgtgggatgaacttaagggctcggacaacatcattcgtactctgcaatggactgctcgagtggagggaagcgatcagtacaccacctctaagcatgccatgactctcagccaatatctcggcagaggtgttgtttccagaggccggatggcaatttcatcgggtctggacaccaatgtggccgagcacccgtacctccacaacgatgtcgacaagcagaccgtcatccaaggcatcaagaacctccaggcggcgctgaatgtcattcccaacctttcctgggttttgcctcccccgaacacgactgtcgagtcatttatcaacaatgtgagttctccttttctgtttatcgctgtctgagccataccttttactgacatatcggtgtctgtagatgatcgtctcaccctccaatcgtcggtcaaaccattggatgggaactgccaagcttggcaaggacgatggccgtactggaggcagcgctgtcgtggatctgaacaccaaggtgtacggtaccgataacctctttgttgttgacgcctccatcttccctggtatgaccaccggcaacccgtcggcgatgatcgtgattgcctcggagcatgctgcacagaaaatcttggctttgaagcctgtcccatctctgcctggcggcaatggcaagggaaaatggagaagatga

SEQ ID NO: 42 - DNA, 2487 bp, Neurospora crassa
atgaaggtcttcacccgcattggaacgatcgttctggcgacgtcactgtacctacagcaatgctccgctcaatacatcaacgagcaatataccgatcccgtgaacaagatcaccctcagcacctggcggccagaccctggttctaattctgggggtggagatgctgccacctacgcctttggcttggtcttgcctccggatgctctgaccaaagatgccaacgaatacatcggtctcttgcgctgtgatgttggtgatgcggcgagccccggatggtgtggtgtctcccacggccagtctggacaaatgacacagtcgttgttgctcatggcttgggcctccaagggtcaagtctttacctcatttcgctacgcatccggttataatgtgccaggactctacaccggaaatgcaaccctgacccagatctctgccactgtgaactcgacacagttcgaattgatctatcgctgccaggactgttttgcatggaaccaaggaggaagcaagggaagcgtatcaaccagcagtggccttctcgtcttgggccgtgccgcggccaagggaaatcttcagaacccgacttgccctgacaaggccattcccggctttcatgacaatgggtttggtcaatatggagcgcctctcgagaaagtcccgcatacctcatactcagcttgggcttctttagccacgaagaccactactgctgactgctctggggcatccgacccagtacccactggatccgagccgccagccgagccaacttcgacagcggagcccgttcccgtttgcacacctgccccaagcaagacgtacgactacatcatcgttggcgccggtgctggtggcattcccattgcggacaagctcagcgaggccggaaaaagtgtgttgttgatcgaaaagggacctccctccactggaagatggaagggcaccatgaagcctgagtggcttcagggcacgaacttgactcgcttcgatgttcctggtctatgcaaccagatctgggtggactctgccggcatcgcctgtacagataccgaccaaatggcgggatgtgtcctgggcggaggaacggctgttaatgccggcctgtggtggaagccgcatcctcaggattggaactacaacttccccgagggctggaagtcgagagataccgtgccagccactaaccgtgtgttcggtcgcattcctggaacttggcatccttcgcaaaacggcaagctgtaccgacaagagggcttcaacgtcctagccagcgggctgagcaagagcggttggaaggaggtgatccccaacgatgcatacaaccagaagaaccacacctttggtcacagcaccttcatgttcgctaaaggcgagcgaggtggccctctggcaacataccttgtgacggcggtagctcgcaagcagttcactctctggaccaatgtagctgtgagaagggcagttcgtaacggaagccgtatcactggcgttgagctcgaatgcttgacggatggtggtctcagcggaactgtcaacgtgacccctaacactggccgtgttatctttgctgcaggcacttttggttccgccaagcttctccttcgcagcggtatcggacctaccgatcaactcgagattgtcaaggggtcgacggatggcccaacgttcatttccaaggaccaatggatcaaccttccagttggctacaacctcatggatcatctcaacactgatctcattatcacccatcctgacgttgtcttctacgacttctacgaggcttggaacacgcccattgaaggtgacaagagcgcctatcttcagaatagatctggaatccttgcccaggctgctcccaatattggtcctttgatgtgggatgaacttaagggctcggacaacatcattcgtactctgcaatggactgctcgagtggagggaagcgatcagtacaccacctctaagcatgccatgactctcagccaatatctcggcagaggtgttgtttccagaggccggatggcaatttcatcgggtctggacaccaatgtggccgagcacccgtacctccacaacgatgtcgacaagcagaccgtcatccaaggcatcaagaacctccaggcggcgctgaatgtcattcccaacctttcctgggttttgcctcccccgaacacgactgtcgagtcatttatcaacaatatgatcgtctcaccctccaatcgtcggtcaaaccattggatgggaactgccaagcttggcaaggacgatggccgtactggaggcagcgctgtcgtggatctgaacaccaaggtgtacggtaccgataacctctttgttgttgacgcctccatcttccctggtatgaccaccggcaacccgtcggcgatgatcgtgattgcctcggagcatgctgcacagaaaatcttggctttgaagcctgtcccatctctgcctggcggcaatggcaagggaaaatggagaagatga

SEQ ID NO: 43 - protein, 828 aa, Neurospora crassa
MKVFTRIGTIVLATSLYLQQCSAQYINEQYTDPVNKITLSTWRPDPGSNSGGGDAATYAFGLVLPPDALTKDANEYIGLLRCDVGDAASPGWCGVSHGQSGQMTQSLLLMAWASKGQVFTSFRYASGYNVPGLYTGNATLTQISATVNSTQFELIYRCQDCFAWNQGGSKGSVSTSSGLLVLGRAAAKGNLQNPTCPDKAIPGFHDNGFGQYGAPLEKVPHTSYSAWASLATKTTTADCSGASDPVPTGSEPPAEPTSTAEPVPVCTPAPSKTYDYIIVGAGAGGIPIADKLSEAGKSVLLIEKGPPSTGRWKGTMKPEWLQGTNLTRFDVPGLCNQIWVDSAGIACTDTDQMAGCVLGGGTAVNAGLWWKPHPQDWNYNFPEGWKSRDTVPATNRVFGRIPGTWHPSQNGKLYRQEGFNVLASGLSKSGWKEVIPNDAYNQKNHTFGHSTFMFAKGERGGPLATYLVTAVARKQFTLWTNVAVRRAVRNGSRITGVELECLTDGGLSGTVNVTPNTGRVIFAAGTFGSAKLLLRSGIGPTDQLEIVKGSTDGPTFISKDQWINLPVGYNLMDHLNTDLIITHPDVVFYDFYEAWNTPIEGDKSAYLQNRSGILAQAAPNIGPLMWDELKGSDNIIRTLQWTARVEGSDQYTTSKHAMTLSQYLGRGVVSRGRMAISSGLDTNVAEHPYLHNDVDKQTVIQGIKNLQAALNVIPNLSWVLPPPNTTVESFINNMIVSPSNRRSNHWMGTAKLGKDDGRTGGSAVVDLNTKVYGTDNLFVVDASIFPGMTTGNPSAMIVIASEHAAQKILALKPVPSLPGGNGKGKWRR

SEQ ID NO: 44 - DNA, 2953 bp, Methanosaeta thermophila
atgaggacctcctctcgtttaatcggtgcccttgcggcggcacgtaagtcagagcttagcgtggctcacggtccttcctgtcactaacttgcctgctttgtagtcttgccgtctgcccttgcgcagaacaacgcgccggtaaccttcaccgacccggactcgggcattaccttcaacacgtggggtctcgccgaggattctccccagactaagggcggtttcacttttggtgttgctctgccctctgatgccctcacgacagacgccaaggagttcatcggttacttggtaagccatgtccgagacgcacatgccactcacagctgctaaccgccccagaaatgcgcgaggaacgatgagagcggttggtgcggtgtctccctgggcggccccatgaccaactcgctcctcatcgcggcctggccccacgaggacaccgtctacacctctctccgcttcgccaccggctatgccatgccggatgtctaccagggggacgccgagatcacccaggtctcctcctctgtcaactcgacgcacttcagcctcatcttcaggtgcgagaactgcctgcaatggagtcaaagcggcgccaccggcggtgcctccacctcgaacggcgtgttggtcctcggctgggtccaggcattcgccgaccccggcaacccgacctgccccgaccagatcaccctcgagcagcacgacaacggcatgggtatctggggtgcccagctcaactccgacgccgccagcccgtcctacaccgagtgggccgcccaggccaccaagaccgtcacgggtgactgcggcggtcccaccgagacctctgtcgtcggtgtccccgttccgacgggcgtctcgttcgattacatcgtcgtgggcggcggtgccggtggcatccccgccgccgacaagctcagcgaggccggcaagagtgtgctgctcatcgagaagggctttgcctcgaccgccaacaccggaggcactctcggccccgagtggctcgagggccacgaccttacccgctttgacgtgccgggtctgtgcaaccagatctgggttgactccaaggggatcgcttgcgaggataccgaccagatggctggctgtgtcctcggcggcggtaccgccgtgaatgccggcctgtggttcaagccctactcgctcgactgggactacctcttccctagtggttggaagtacaaagacgtccagccggccatcaaccgcgccctctcgcgcatcccgggcaccgatgctccctcgaccgacggcaagcgctactaccaacagggcttcgacgtcctctccaagggcctggccggcggcggctggacctcggtcacggccaataacgcgccagacaagaagaaccgcaccttctcccatgcccccttcatgttcgccggcggcgagcgcaacggcccgctgggcacctacttccagaccgccaagaagcgcagcaacttcaagctctggctcaacacgtcggtcaagcgcgtcatccgccagggcggccacatcaccggcgtcgaggtcgagccgttccgcgacggcggttaccaaggcatcgtccccgtcaccaaggttacgggccgcgtcatcctctctgccggtacctttggcagtgcaaagatcctgctgaggagcggtatcggtccgaacgatcagctgcaggttgtcgcggcctcggagaaggatggccctaccatgatcagcaactcgtcctggatcaacctgcctgtcggctacaacctggatgaccacctcaacgtaagtttcagaacacaagagttggtcagtgacaaaatactgcgaagcgaaccgctgacccccttcggtagaccgacactgtcatctcccaccccgacgtcgtgttctacgacttctacgaggcgtgggacaatcccatccagtctgacaaggacagctacctcaactcgcgcacgggcatcctcgcccaagccgctcccaacattgggcctatgtgagtccggcgagctcaagcctgtttgtgttcccctaactaaccgaagccaacaaggttctgggaagagatcaagggtgcggacggcattgttcgccagctccagtggactgcccgtgtcgagggcagcctgggtgcccccaacggcagtacgtagattcctttttttttttttttttttttcatcgactaatccccacgctaactttgtccgtccgctctccagagaccatgaccatgtcgcagtacctcggtcgtggtgccacctcgcgcggccgcatgaccatcaccccgtccctgacaactgtcgtctcggacgtgccctacctcaaggaccccaacgacaaggaggccgtcatccagggcatcatcaacctgcagaacgccctcaagaacgtcgccaacctgacctggctcttccccaactcgaccatcacgccgcgccaatacgttgacagcgtaagtttttgtttacactcctctcccccatccctcccccttcagattgcacttttacttcctctcaaaagagggagaaagagagagcttgcaaggacaattccatactgacataacccttcttcccccttccccctcccctttctccagatggtcgtctccccgagcaaccggcgctccaaccactggatgggcaccaacaagatcggcaccgacgacgggcgcaagggcggctccgccgtcgtcgacctcaacaccaaggtctacggcaccgacaacctcttcgtcatcgacgcctccatcttccccggcgtgcccaccaccaaccccacctcgtacatcgtgacggcgtcggagcacgcctcggcccgcatcctcgccctgcccgacctcacgcccgtccccaagtacgggcagtgcggcggccgcgaatggagcggcagcttcgtctgcgccgacggctccacgtgccagatgcagaacgagtggtactcgcagtgcttgtga

SEQ ID NO: 45 - DNA, 2487 bp, Methanosaeta thermophila
atgaggacctcctctcgtttaatcggtgcccttgcggcggcactcttgccgtctgcccttgcgcagaacaacgcgccggtaaccttcaccgacccggactcgggcattaccttcaacacgtggggtctcgccgaggattctccccagactaagggcggtttcacttttggtgttgctctgccctctgatgccctcacgacagacgccaaggagttcatcggttacttgaaatgcgcgaggaacgatgagagcggttggtgcggtgtctccctgggcggccccatgaccaactcgctcctcatcgcggcctggccccacgaggacaccgtctacacctctctccgcttcgccaccggctatgccatgccggatgtctaccagggggacgccgagatcacccaggtctcctcctctgtcaactcgacgcacttcagcctcatcttcaggtgcgagaactgcctgcaatggagtcaaagcggcgccaccggcggtgcctccacctcgaacggcgtgttggtcctcggctgggtccaggcattcgccgaccccggcaacccgacctgccccgaccagatcaccctcgagcagcacgacaacggcatgggtatctggggtgcccagctcaactccgacgccgccagcccgtcctacaccgagtgggccgcccaggccaccaagaccgtcacgggtgactgcggcggtcccaccgagacctctgtcgtcggtgtccccgttccgacgggcgtctcgttcgattacatcgtcgtgggcggcggtgccggtggcatccccgccgccgacaagctcagcgaggccggcaagagtgtgctgctcatcgagaagggctttgcctcgaccgccaacaccggaggcactctcggccccgagtggctcgagggccacgaccttacccgctttgacgtgccgggtctgtgcaaccagatctgggttgactccaaggggatcgcttgcgaggataccgaccagatggctggctgtgtcctcggcggcggtaccgccgtgaatgccggcctgtggttcaagccctactcgctcgactgggactacctcttccctagtggttggaagtacaaagacgtccagccggccatcaaccgcgccctctcgcgcatcccgggcaccgatgctccctcgaccgacggcaagcgctactaccaacagggcttcgacgtcctctccaagggcctggccggcggcggctggacctcggtcacggccaataacgcgccagacaagaagaaccgcaccttctcccatgcccccttcatgttcgccggcggcgagcgcaacggcccgctgggcacctacttccagaccgccaagaagcgcagcaacttcaagctctggctcaacacgtcggtcaagcgcgtcatccgccagggcggccacatcaccggcgtcgaggtcgagccgttccgcgacggcggttaccaaggcatcgtccccgtcaccaaggttacgggccgcgtcatcctctctgccggtacctttggcagtgcaaagatcctgctgaggagcggtatcggtccgaacgatcagctgcaggttgtcgcggcctcggagaaggatggccctaccatgatcagcaactcgtcctggatcaacctgcctgtcggctacaacctggatgaccacctcaacaccgacactgtcatctcccaccccgacgtcgtgttctacgacttctacgaggcgtgggacaatcccatccagtctgacaaggacagctacctcaactcgcgcacgggcatcctcgcccaagccgctcccaacattgggcctatgttctgggaagagatcaagggtgcggacggcattgttcgccagctccagtggactgcccgtgtcgagggcagcctgggtgcccccaacggcaagaccatgaccatgtcgcagtacctcggtcgtggtgccacctcgcgcggccgcatgaccatcaccccgtccctgacaactgtcgtctcggacgtgccctacctcaaggaccccaacgacaaggaggccgtcatccagggcatcatcaacctgcagaacgccctcaagaacgtcgccaacctgacctggctcttccccaactcgaccatcacgccgcgccaatacgttgacagcatggtcgtctccccgagcaaccggcgctccaaccactggatgggcaccaacaagatcggcaccgacgacgggcgcaagggcggctccgccgtcgtcgacctcaacaccaaggtctacggcaccgacaacctcttcgtcatcgacgcctccatcttccccggcgtgcccaccaccaaccccacctcgtacatcgtgacggcgtcggagcacgcctcggcccgcatcctcgccctgcccgacctcacgcccgtccccaagtacgggcagtgcggcggccgcgaatggagcggcagcttcgtctgcgccgacggctccacgtgccagatgcagaacgagtggtactcgcagtgcttgtga

SEQ ID NO: 46 - protein, 828 aa, Methanosaeta thermophila
MRTSSRLIGALAAALLPSALAQNNAPVTFTDPDSGITFNTWGLAEDSPQTKGGFTFGVALPSDALTTDAKEFIGYLKCARNDESGWCGVSLGGPMTNSLLIAAWPHEDTVYTSLRFATGYAMPDVYQGDAEITQVSSSVNSTHFSLIFRCENCLQWSQSGATGGASTSNGVLVLGWVQAFADPGNPTCPDQITLEQHDNGMGIWGAQLNSDAASPSYTEWAAQATKTVTGDCGGPTETSVVGVPVPTGVSFDYIVVGGGAGGIPAADKLSEAGKSVLLIEKGFASTANTGGTLGPEWLEGHDLTRFDVPGLCNQIWVDSKGIACEDTDQMAGCVLGGGTAVNAGLWFKPYSLDWDYLFPSGWKYKDVQPAINRALSRIPGTDAPSTDGKRYYQQGFDVLSKGLAGGGWTSVTANNAPDKKNRTFSHAPFMFAGGERNGPLGTYFQTAKKRSNFKLWLNTSVKRVIRQGGHITGVEVEPFRDGGYQGIVPVTKVTGRVILSAGTFGSAKILLRSGIGPNDQLQVVAASEKDGPTMISNSSWINLPVGYNLDDHLNTDTVISHPDVVFYDFYEAWDNPIQSDKDSYLNSRTGILAQAAPNIGPMFWEEIKGADGIVRQLQWTARVEGSLGAPNGKTMTMSQYLGRGATSRGRMTITPSLTTVVSDVPYLKDPNDKEAVIQGIINLQNALKNVANLTWLFPNSTITPRQYVDSMVVSPSNRRSNHWMGTNKIGTDDGRKGGSAVVDLNTKVYGTDNLFVIDASIFPGVPTTNPTSYIVTASEHASARILALPDLTPVPKYGQCGGREWSGSFVCADGSTCQMQNEWYSQCL

SEQ ID NO: 47 - DNA, 2935 bp, Methanosaeta thermophila
atgaagctactcagccgcgttggggcgaccgccctagcggcgacgttgtgtaagtgtggtcctaacgagccttctcgttgtctcccccggtgaatgctgaggagatgctaatagtcccccaagcactgcagcaatgtgcagcccagatgaccgaggggacctacaccgatgaggctaccggtatccaattcaagacgtggaccgcctccgagggcgcccctttcacgtttggcttgaccctccccgcggacgcgctggaaaaggatgccaccgagtacattggtctcctggtaggttcagcgcggcgccgcaaactggggcttccggctcacctctctcgcagcgttgccaaatcaccgatcccgcctcgcccagctggtgcggtatctcccacggccagtccggccagatgacgcaggcgctgctgctggtcgcctgggccagcgaggacaccgtctacacgtcgttccgctacgccaccggctacacgctccccggcctctacacgggcgacgccaagctgacccagatctcctcctcggtcagcgaggacagcttcgaggtgctgttccgctgcgaaaactgcttctcctgggaccaggatggcaccaagggcaacgtctcgaccagcaacggcaacctggtcctcggccgcgccgccgcgaaggatggtgtgacgggccccacgtgcccggacacggccgagttcggtttccatgataacggtttcggacagtggggtgccgtgcttgagggtgctacttcggactcgtacgaggagtgggctaagctggccacgaccacgcccgagaccacctgcgatgggtaagtgtgctctttttcctctatccgggaaagcgtacagttgctgactcatgtcagcactggccccggcgacaaggagtgcgttccggctcccgaggacacgtatgattacatcgttgtcggtgccggcgccggtggtatcaccgtcgccgacaagctcagcgaggccggccacaaggtccttctcatcgagaagggacccccttcgaccggcctgtggaacgggaccatgaagcccgagtggctcgagagcaccgaccttacccgcttcgacgttcccggcctgtgcaaccagatctgggtcgactctgccggcatcgcctgcaccgataccgaccagatggcgggctgcgttctcggcggtggcaccgctgtcaacgctggtttgtggtggaaggtaaggtttctcgtcagaagaaaccgagtccacgcgcccagatattatattggaacccaggacaagcaccgctaacattacatcgcagccccaccccgctgactgggatgagaacttccccgaagggtggaagtcgagcgatctcgcggatgcgaccgagcgtgtcttcaagcgcatccccggcacgtcgcacccgtcgcaggacggcaagttgtaccgccaggagggcttcgaggtcatcagcaagggcctggccaacgccggctggaaggaaatcagcgccaacgaggcgcccagcgagaagaaccacacctatgcacacaccgagttcatgttctcgggcggtgagcgtggcggccccctggcgacgtaccttgcctcggctgccgagcgcagcaacttcaacctgtggctcaacactgccgtccggagggccgtccgcagcggcagcaaggtcaccggcgtcgagctcgagtgcctcacggacggtggcttcagcgggaccgtcaacctgaatgagggcggtggtgtcatcttctcggccggcgctttcggctcggccaagctgctccttcgcagtaagttttttttttaggtttctttttttttatttttttgcccgcggccacttcgctctctctctctctctctctctctccccctcttctttccctgtgcgaccgcatcaactgacccgatttctctaggcggtatcggtcctgaggaccagctcgagattgtggcgagctccaaggacggcgagaccttcactcccaaggacgagtggatcaacctccccgtcggccacaacctgatcgaccatctcaacactgacctcattatcacgcacccggatgtcgttttctatgacttctatgcggcctgggacgagcccatcacggaggataaggaggcctacctgaactcgcggtccggcattctcgcccaggcggcgcccaatatcggccctatggtaagccttctgacgcccgcgctgagattcatggggtcgttgttcttctgggataaaaataggactgaccgtgttgcacacagatgtgggatcaagtcacgccgtccgacggcatcacccgccagttccagtggacatgccgtgttgagggcgacagctccaagaccaactcgacccgtaagaaccatcccccccttttctcattttctatcaacctggacgtggctttgtttttgtactgactgtccttccttcctctcccagacgccatgaccctcagccagtacctcggccgtggcgtcgtctcgcgcggccggatgggcatcacctccgggctgagcacgacggtggccgagcacccgtacctgcacaacaacggcgacctggaggcggtcatccaggggatccagaacgtggtggacgcgctcagccaggtggccgacctcgagtgggtgctcccgccgcccgacgggacggtggccgactacgtcaacagcctgatcgtctcgccggccaaccgccgggccaaccactggatgggcacggccaagctgggcaccgacgacggccgctcgggcggcacctcggtcgtcgacctcgacaccaaggtgtacggcaccgacaacctgttcgtcgtcgacgcgtccgtcttccccggcatgtcgacgggcaacccgtcggccatgatcgtcatcgtggccgagcaggcggcgcagcgcatcctggccctgcggtcttaa

SEQ ID NO: 48 - DNA, 2364 bp, Methanosaeta thermophila
atgaagctactcagccgcgttggggcgaccgccctagcggcgacgttgtcactgcagcaatgtgcagcccagatgaccgaggggacctacaccgatgaggctaccggtatccaattcaagacgtggaccgcctccgagggcgcccctttcacgtttggcttgaccctccccgcggacgcgctggaaaaggatgccaccgagtacattggtctcctgcgttgccaaatcaccgatcccgcctcgcccagctggtgcggtatctcccacggccagtccggccagatgacgcaggcgctgctgctggtcgcctgggccagcgaggacaccgtctacacgtcgttccgctacgccaccggctacacgctccccggcctctacacgggcgacgccaagctgacccagatctcctcctcggtcagcgaggacagcttcgaggtgctgttccgctgcgaaaactgcttctcctgggaccaggatggcaccaagggcaacgtctcgaccagcaacggcaacctggtcctcggccgcgccgccgcgaaggatggtgtgacgggccccacgtgcccggacacggccgagttcggtttccatgataacggtttcggacagtggggtgccgtgcttgagggtgctacttcggactcgtacgaggagtgggctaagctggccacgaccacgcccgagaccacctgcgatggcactggccccggcgacaaggagtgcgttccggctcccgaggacacgtatgattacatcgttgtcggtgccggcgccggtggtatcaccgtcgccgacaagctcagcgaggccggccacaaggtccttctcatcgagaagggacccccttcgaccggcctgtggaacgggaccatgaagcccgagtggctcgagagcaccgaccttacccgcttcgacgttcccggcctgtgcaaccagatctgggtcgactctgccggcatcgcctgcaccgataccgaccagatggcgggctgcgttctcggcggtggcaccgctgtcaacgctggtttgtggtggaagccccaccccgctgactgggatgagaacttccccgaagggtggaagtcgagcgatctcgcggatgcgaccgagcgtgtcttcaagcgcatccccggcacgtcgcacccgtcgcaggacggcaagttgtaccgccaggagggcttcgaggtcatcagcaagggcctggccaacgccggctggaaggaaatcagcgccaacgaggcgcccagcgagaagaaccacacctatgcacacaccgagttcatgttctcgggcggtgagcgtggcggccccctggcgacgtaccttgcctcggctgccgagcgcagcaacttcaacctgtggctcaacactgccgtccggagggccgtccgcagcggcagcaaggtcaccggcgtcgagctcgagtgcctcacggacggtggcttcagcgggaccgtcaacctgaatgagggcggtggtgtcatcttctcggccggcgctttcggctcggccaagctgctccttcgcagcggtatcggtcctgaggaccagctcgagattgtggcgagctccaaggacggcgagaccttcactcccaaggacgagtggatcaacctccccgtcggccacaacctgatcgaccatctcaacactgacctcattatcacgcacccggatgtcgttttctatgacttctatgcggcctgggacgagcccatcacggaggataaggaggcctacctgaactcgcggtccggcattctcgcccaggcggcgcccaatatcggccctatgatgtgggatcaagtcacgccgtccgacggcatcacccgccagttccagtggacatgccgtgttgagggcgacagctccaagaccaactcgacccacgccatgaccctcagccagtacctcggccgtggcgtcgtctcgcgcggccggatgggcatcacctccgggctgagcacgacggtggccgagcacccgtacctgcacaacaacggcgacctggaggcggtcatccaggggatccagaacgtggtggacgcgctcagccaggtggccgacctcgagtgggtgctcccgccgcccgacgggacggtggccgactacgtcaacagcctgatcgtctcgccggccaaccgccgggccaaccactggatgggcacggccaagctgggcaccgacgacggccgctcgggcggcacctcggtcgtcgacctcgacaccaaggtgtacggcaccgacaacctgttcgtcgtcgacgcgtccgtcttccccggcatgtcgacgggcaacccgtcggccatgatcgtcatcgtggccgagcaggcggcgcagcgcatcctggccctgcggtcttaa

SEQ ID NO: 49 - protein, 787 aa, Methanosaeta thermophila
MKLLSRVGATALAATLSLQQCAAQMTEGTYTDEATGIQFKTWTASEGAPFTFGLTLPADALEKDATEYIGLLRCQITDPASPSWCGISHGQSGQMTQALLLVAWASEDTVYTSFRYATGYTLPGLYTGDAKLTQISSSVSEDSFEVLFRCENCFSWDQDGTKGNVSTSNGNLVLGRAAAKDGVTGPTCPDTAEFGFHDNGFGQWGAVLEGATSDSYEEWAKLATTTPETTCDGTGPGDKECVPAPEDTYDYIVVGAGAGGITVADKLSEAGHKVLLIEKGPPSTGLWNGTMKPEWLESTDLTRFDVPGLCNQIWVDSAGIACTDTDQMAGCVLGGGTAVNAGLWWKPHPADWDENFPEGWKSSDLADATERVFKRIPGTSHPSQDGKLYRQEGFEVISKGLANAGWKEISANEAPSEKNHTYAHTEFMFSGGERGGPLATYLASAAERSNFNLWLNTAVRRAVRSGSKVTGVELECLTDGGFSGTVNLNEGGGVIFSAGAFGSAKLLLRSGIGPEDQLEIVASSKDGETFTPKDEWINLPVGHNLIDHLNTDLIITHPDVVFYDFYAAWDEPITEDKEAYLNSRSGILAQAAPNIGPMMWDQVTPSDGITRQFQWTCRVEGDSSKTNSTHAMTLSQYLGRGVVSRGRMGITSGLSTTVAEHPYLHNNGDLEAVIQGIQNVVDALSQVADLEWVLPPPDGTVADYVNSLIVSPANRRANHWMGTAKLGTDDGRSGGTSVVDLDTKVYGTDNLFVVDASVFPGMSTGNPSAMIVIVAEQAAQRILALRS

SEQ ID NO: 50 - protein, 722 aa, Coprinopsis cinerea
MFSSLFWAIGLLSVLVHGQVASQWYDSLTGVTWQRYYQQDFDASWGYLFPSSAGGAATDEFIGIFQAPANSGWIGNSLGGGMRNAPLIVGWVDGTTPRISARWATDYAPPSIYSGPRLTILGSSGSNGQIQRIVYRCQNCTSWSGGGIPSTGSSVLGWAFHATLQPLTPSDPNSGLYRHSAAGQHGFDLGTRTSSYNYFLQQLTNAPPLSGGAPTQPPTSQPPTPTTPPPQPPPSSTFVSCPGAPNPRYPINVVSGWRAVPVLGSLSEPRGITMDTRGNLLVLQRGRGLSGHTLDANGCVTSSKMVIQDSAINHGVDVHPAGNRIIASSGDIAWSWDYDPVTMTTSNKRTLVTGMNNNFHFTRTILISKKNPNIFAINVGSASNIDEPTRQPGSGRAQIRVFDYNNLPASGTTFTSSYGRVLGYGLRNDVGIAQDRAGNFWSIENSLDDAYRMINGQRRDIHINNPAEKVYNLGDPANPRSLFGGYPDCYTIWEPADFNDSTKRVGDWFTQTNSGQYNDAYCNSNTTAKPVVLLPPHTAPLDFKFGVGNDSNLYVPLHGSWNRQPPQGYKVVIVPGRWSASGEWSPTVSLAETKNSWSTLISNVDETRCSGFGNANCFRPVGLVFSPDGQNLYVTSDSSGEVILVKRLSGPTNPGQPPTITTQPGTPTSQPPVQPPTTIAPPQATQTMYGQCGGQGWTGPTLCPANAVCRASNQWYSQCVPA

SEQ ID NO: 51 - protein, 342 aa, Coprinopsis cinera
PGAPNPRYPINVVSGWRAVPVLGSLSEPRGITMDTRGNLLVLQRGRGLSGHTLDANGCVTSSKMVIQDSAINHGVDVHPAGNRIIASSGDIAWSWDYDPVTMTTSNKRTLVTGMNNNFHFTRTILISKKNPNIFAINVGSASNIDEPTRQPGSGRAQIRVFDYNNLPASGTTFTSSYGRVLGYGLRNDVGIAQDRAGNFWSIENSLDDAYRMINGQRRDIHINNPAEKVYNLGDPANPRSLFGGYPDCYTIWEPADFNDSTKRVGDWFTQTNSGQYNDAYCNSNTTAKPVVLLPPHTAPLDFKFGVGNDSNLYVPLHGSWNRQPPQGYKVVIVPGRWSASGE

SEQ ID NO: 52 - protein, 238 aa, Sordaria macrospora
MKVLAPLVLASAASAHTIFSSLEVGGVNQGLGQGVRVPTYNGPIEDVTSASIACNGSPNTVGSTSKVITVQAGTNVTAIWRYMLSTTGDSPADVMDSTHKGPTIAYLKKVDNAATDSGVGNGWFKIQQDGMDANGVWGTERVINGKGRQSIKIPECIAPGQYLLRAEMIALHSAGNYPGAQFYMECAQLNVVGGTGAKTPSTVSFPGAYSGSDPGVKINIYWPPVTSYTVPGPSVFTC

SEQ ID NO: 53 - protein, 238 aa, Glomerella graminicola
MKVLLPLLTASLASAHTIFSSLEVGGVNQGIGGGVRVPSYNGPIENVQSDSLACNGAPNPTTPTSKVITVQAGQNVTAIWRYMLSSTGSGPADVMDSTHKGPTIAYLKKVNDATSDSGIGSGWFKIQQDGYNNGVWGTEKVINGQGRHSIKIPECIAPGQYLLRAEMIALHAAGSYPGAQFYMECAQINVVGGTGSKTPSSTVSFPGAYKSSDPGVTISIYWPPVTTYTIPGPALFTC

SEQ ID NO: 54 - protein, 238 aa, Chaetomium globosum
MKVLAPLMLAGAASAHTIFSSLEVGGVNQGVGQGVRVPSYNGPIEDVTSNSMACNGNPNPTSSTSKIITVQAGQSVTAVWRYMLSTTGSAPNDVMDSSHKGPTLAYLKKVGDATSDSGVGGGWFKIQQDGYSNGVWGTEKVINGQGRHTIKIPECIAPGQYLLRAEMIALHGAGNYPGAQFYMECAQINVVGGSGSKTPSNTVSFPGAYKGTDPGVKISIYWPPVENYQIPGPSVFTC

SEQ ID NO: 55 - protein, 236 aa, Podospora anserina
MKFAPILLASAASAHTIFSSLEVNGVNHGVGGGVRVPSYNGPIENVDSASIACNGAPNPTTPTSKVITVQAGQNVTAIWRYMLSTTGSAPNDIMDISHKGPTMAYLKKVNDATTDSGVGGGWFKIQEDGYNNGVWGTEKVINGQGRHSIKIPSCIAPGQYLLRAEMLALHGAGNYPGAQFYMECAQLNIVGGTGSKTPSTVAFPGAYSGSHPGVKISIYWPPVTNYQIPGPSVFTC

SEQ ID NO: 56 - protein, 234 aa, Glomerella graminicola
MRLLNLLAAAGFCQAHTIFVSLDADGVNSGISQGVRTPDYDGPQTDVTSQYIACNGPPNPTKPTDKVITVTAGSTVTAIWRHTLTSGPDDVMDASHKGPTIAYLKKVNDAKTDTGVGGGWYKIQEDGFSNGVWGTERVINNAGKHNITIPKCIANGQYLLRAEMIALHSASSYPGAQLYMECAQINVVGGTAAKTPSTVSFPGAYKGTDPGITLSIYYPPVTNYVIPGPQKFSC

SEQ ID NO: 57 - protein, 322 aa, Sordaria macrospora
MKVLSLLAAASAASAHTIFVQLEAGGTTYPVSHGIRTPSYDGPITDVTSNDLACNGGPNPTTPSDKIMTVNAGSTVKAIWRHTLTSGPSDVMDASHKGPTLAYLKKVDNALTDSGIGGGWFKIQEDGYNNGQWGTSTVITNGGFHYIDIPACITNGQYLLRAEMIALHAASSTAGAQLYMECAQINIVGGTGTASPSTYSIPGIYKANDPGLLVNIYSMGTSSAYTIPGPAKFTCSGSGNGGGSPAPGTTTTAKPVVSSTTTSKAAATTSSTTLKTSVVPSQPTGCTAAQWAQCGGVGFSGCTTCASPYTCKKQNDYYSQCS

SEQ ID NO: 58 - protein, 239 aa, Moniliophthora perniciosa
MKAIILLALTASASAHTIFQQLYVNGEDQGHLEGIRVPDYDGPIQDVTSNDFICNGGINPYHQPISQTVIQVPAGAEVTAEWHHTLDGATGAADDVIDASHKGPIITYLAKVNDATSLDVTGLQWFKIYEDGYDASSGTWAVDKLIANQGKVSFKIPDCIPAGQYLMRHELIALHAAGSYPGAQFYMECAQLEITGGGSASPATVSFPGAYAGSDPGITINIYQSLTRYTIPGPEVFAC

SEQ ID NO: 59 - protein, 235 aa, Schizophyllum commune
LSAALFVGGASAHTIFQKMYVDGVDQGQLTGIRVPDYDGPISDVTSNDIICNGGINPYHQPVSTDVITVPAGSQVTAEWHHTLNGADASDAADPIDASHKGPVISYLAKVDDPTKLDATGLSWFKIHEEGYDPSSNTWGVDTMIKNKGKVTFEIPSCIEDGFYLLRHELIALHGASNYPGAQFYMECAQIEVTGGSGSASPKTVSFPGAYSGSDPGIKINIYQTLNSYTIPGVFT

SEQ ID NO: 60 - protein, 321 aa, Sclerotinia sclerotiorum
MKLQFLIPSSFLLSYVSAHTIFTQLESGGTLYNTSYAIRDPTYDGPITDVTTQYVACNGGPNPTTPSSNIINVVAGSTVKAIWRHTLTSTPSNDATYVLDPSHLGPVMAYMKKVDDATTDVGYGPGWFKISEQGLNVATQGWATTDLINNAGVQSITIPSCIANGQYLLRAELIALHAASGLQGAQLYMECAQINVSGGTGTSSPSTVSFPGAYAQNDPGILINIYQTLSSYPIPGPTPFVCGAAQSTAKSSTSTSLSSTAKATSTTLVTSTKSSSSVLATGTAVAAIYAQCGGQGWNGATTCAAGSKCVVSSAYYSQCLP

SEQ ID NO: 61 - protein, 322 aa, Coprinopsis cinerea
MKNLFSLATLAVLLSSVSAHTIFQELHVNGVRQGRTVGIRVPYYNGPIENVNSNDIICNGGINPYKTPISQTVIPVPAGATVTAEWRYTLDSKPGDNSDPIDPSHKGPILAYLAKVPSATQSNVTGLKWFKIYHDGYDAATNTWAVDKLIRDQGLVSFKIPDCIEDGDYLLRVELIALHSASSYPGAQFYMECAQIRISGGGNVTPSNTVSFPGAYSGSDPGVRINIYQGVRSYTIPGPSVWTCPAGSGPGNPAPTTPAPPVVPTTVAPPPVQTTAPPTTPPSQGTVPQWGQCGGNGYSGPTECVAPFRCVKTNDWYSQCVA

SEQ ID NO: 62 - protein, 310 aa, Volvariella volvacea
MKSFFKLASLVLLAQSVAAHTIFQELHVNGVSQGHINGIRVPDYDGPITDVTSNDIICNGGINPYHQPISTTIINVPAGAQVTAEFHHTLQGANPSDSSDPIDSSHKGPILAYLAKVDNALTPNVTGLKWFKIYHDGLSNGVWAVDKLITNKGKVTFTIPNCIPPGHYLLRVELIALHAAGSYPGAQFYMECAQINITGGGNTTPANTVSFPGAYSGSDPGVKVNIYSGLTSYVIPGPPVWTCSGNNTPNPTTSQPPSSTSVPTSTPPTSTPVGTVPQWGQCGGIGYNGPTVCVSPFTCTKVNDYYSQYL

SEQ ID NO: 63 - protein, 300 aa, Podospora anserina
MKFLSLLAAASTATAHTIFVQLDAGGKVYPVSHAIRTPTYDGPITNVNSNDLACNGGPNPTMKSNEVITVQAGTTVKAVWRHTLTSGPNNVMDASHKGPTLAYLKKVSNALTDTGIGGGWFKIQEDGYNGGNWGTSKVINNAGLHYMFVSPPPPPFFFFSFFLSLLYELSWLISMECAQINIVGGTGAVSPKTYSIPGIYKSNDPGILVNIYSMTTSSKYTIPGPPLFTCAGGSGGSGPVTTQPEPVVEEVPVPTQPEPVDSGCEAAQWQQCGGQNYSGCTRCAAGFTCKNINQYYHQCS

SEQ ID NO: 64 - protein, 359 aa, Neurospora crassa
MKTGSILAALVASASAHTIFQKVSVNGADQGQLKGIRAPANNNPVTDVMSSDIICNAVTMKDSNVLTVPAGAKVGHFWGHEIGGAAGPNDADNPIAASHKGPIMVYLAKVDNAATTGTSGLKWFKVAEAGLSNGKWAVDDLIANNGWSYFDMPTCIAPGQYLMRAELIALHNAGSQAGAQFYIGCAQINVTGGGSASPSNTVSFPGAYSASDPGILINIYGGSGKTDNGGKPYQIPGPALFTCPAGGSGGSSPAPATTASTPKPTSASAPKPVSTTASTPKPTNGSGSGTGAAHSTKCGGSKPAATTKASNPQPTNGGNSAVRAAALYGQCGGKGWTGPTSCASGTCKFSNDWYSQCLP

SEQ ID NO: 65 - protein, 312 aa, Phanerochaete chrysosporium
  VARIANT 101: Xaa = Any Amino Acid
LAAVALSSSAHTIFQEVYVNGVDQGHINGIRVPTYDGPVTDVTSNGIICNGVENPFQQPVSDVIITVPAGATVTAEWHHTLAGADPSDPADPVDPSHKGEXPVITYLAQVPNALQTDVTGLKWFKIWEDGLDVSDQSWGVDRMIANKGKVTFTIPDCIPAGQYLMRHEMIALHGAESYPGAQFYMECAQLQITGGGSTQPATVSFPGAYSGTDPGIKINIYQTLKNYTIPGPPVFSCDGSTALPPPPPPATSTAAPHTSSAPSASSAAPPPPTATATAGHYAQCGGIGYTGPTVCAAPYTCTVSNEYYSQCL

SEQ ID NO: 66 - protein, 317 aa, Thielavia terrestris
MKGLSLLAAASAATAHTIFVQLESGGTTYPVSYGIRDPSYDGPITDVTSDSLACNGPPNPTTPSPYIINVTAGTTVAAIWRHTLTSGPDDVMDASHKGPTLAYLKKVDDALTDTGIGGGWFKIQEAGYDNGNWATSTVITNGGFQYIDIPACIPNGQYLLRAEMIALHAASTQGGAQLYMECAQINVVGGSGSASPQTYSIPGIYQATDPGLLINIYSMTPSSQYTIPGPPLFTCSGSGNNGGGSNPSGGQTTTAKPTTTTAATTTSSAAPTSSQGGSSGCTVPQWQQCGGISFTGCTTCAAGYTCKYLNDYYSQCQ

SEQ ID NO: 67 - protein, 316 aa, Phanerochaete chrysosporium
LSLVGAALALSASAHTIFQELYVNGVDQGHTVGIRVPSYDGPVTDVTSNGIICNGVENPFTTPISKIVIPVPAGATVTAEWHHTLAGADPSDSADPVDPSHKGPVISYLAQIPDATQSDVTGLKWFKIWEDGLNPADQSWGVDRMIANKGKVTFTIPSCIPSGQYLLRHEMIALHPASSYPGAQFYMECAQLQITGGGSTQPATVSFPGAYHGTDPGIKINIYQHLSNYTIPGPPVFSCDGGSAAPPPPPPPPPPPTSVSSQPSSVSSVPAPPHTSTPTGPTAAHYAQCGGIGYTGPTVCAAPYTCTVSNAYYSQC

SEQ ID NO: 68 - protein, 235 aa, Sporotrichum thermophile
MKALSLLAAAGAVSAHTIFVQLEADGTRYPVSYGIRDPTYDGPITDVTSNDVACNGGPNPTTPSSDVITVTAGTTVKAIWRHTLQSGPDDVMDASHKGPTLAYIKKVGDATKDSGVGGGWFKIQEDGYNNGQWGTSTVISNGGEHYIDIPACIPEGQYLLRAEMIALHAAGSPGGAQLYMECAQINIVGGSGSVPSSTVSFPGAYSPNDPGLLINIYSMSPSSSYTIPGPPVFKC

SEQ ID NO: 69 - protein, 237 aa, Sporotrichum thermophile
MKVLAPLILAGAASAHTIFSSLEVGGVNQGIGQGVRVPSYNGPIEDVTSNSIACNGPPNPTTPTNKVITVRAGETVTAVWRYMLSTTGSAPNDIMDSSHKGPTMAYLKKVDNATTDSGVGGGWFKIQEDGLTNGVWGTERVINGQGRHNIKIPECIAPGQYLLRAEMLALHGASNYPGAQFYMECAQLNIVGGTGSKTPSTVSFPGAYKGTDPGVKINIYWPPVTSYQIPGPGVFTC

SEQ ID NO: 70 - protein, 182 aa, Neurospora crassa
TFTHPDTGIVFNTWSASDSQTKGGFTVGMALPSNALTTDATEFIGYLECSSAKNGANSGWCGVSLRGAMTNNLLITAWPSDGEVYTNLMFATGYAMPKNYAGDAKITQIASSVNATHFTLVFRCQNCLSWDQDGVTGGISTSNKGAQLGWVQAFPSPGNPTCPTQITLSQHDNGMGQWGAAF

SEQ ID NO: 71 - DNA, 546 bp, Neurospora crassa
accttcactcatcctgataccggcattgtcttcaacacatggagtgcttccgattcccagaccaaaggtggcttcactgttggtatggctctgccgtcaaatgctcttactaccgacgcgactgaattcatcggttatctggaatgctcctccgccaagaatggtgccaatagcggttggtgcggtgtttctctcagaggcgccatgaccaacaatctactcattaccgcctggccttctgacggagaagtctacaccaatctcatgttcgccacgggttacgccatgcccaagaactacgctggtgacgccaagatcacccagatcgcgtccagcgtgaacgctacccacttcacccttgtctttaggtgccagaactgtttgtcatgggaccaagacggtgtcaccggcggcatttctaccagcaataagggggcccagctcggttgggtccaggcgttcccctctcccggcaacccgacttgccctacccagatcactctcagtcagcatgacaacggtatgggccagtggggagctgccttt

SEQ ID NO: 72 - protein, 527 aa, Neurospora crassa
PVPTGVSFDYIVVGGGAGGIPVADKLSESGKSVLLIEKGFASTGEHGGTLKPEWLNNTSLTRFDVPGLCNQIWKDSDGIACSDTDQMAGCVLGGGTAINAGLWYKPYTKDWDYLFPSGWKGSDIAGATSRALSRIPGTTTPSQDGKRYLQQGFEVLANGLKASGWKEVDSLKDSEQKNRTFSHTSYMYINGERGGPLATYLVSAKKRSNFKLWLNTAVKRVIREGGHITGVEVEAFRNGGYSGIIPVTNTTGRVVLSAGTFGSAKILLRSGIGPKDQLEVVKASADGPTMVSNSSWIDLPVGHNLVDHTNTDTVIQHNNVTFYDFYKAWDNPNTTDMNLYLNGRSGIFAQAAPNIGPLFWEEITGADGIVRQLHWTARVEGSFETPDGYAMTMSQYLGRGATSRGRMTLSPTLNTVVSDLPYLKDPNDKAAVVQGIVNLQKALANVKGLTWAYPSANQTAADFVDKQPVTYQSRRSNHWMGTNKMGTDDGRSGGTAVVDTNTRVYGTDNLYVVDASIFPGVPTTNPT

SEQ ID NO: 73 - DNA, 1581 bp, Neurospora crassa
cctgttcccactggcgtttcttttgactacattgtcgttggtggtggtgccggtggtattcccgtcgctgacaagctcagcgagtccggtaagagcgtgctgctcatcgagaagggtttcgcttccactggtgagcatggtggtactctgaagcccgagtggctgaataatacatcccttactcgcttcgatgttcccggtctttgcaaccagatctggaaagactcggatggcattgcctgctccgataccgatcagatggccggctgcgtgctcggcggtggtaccgccatcaacgccggtctctggtacaagccctacaccaaggactgggactacctcttcccctctggctggaagggcagcgatatcgccggtgctaccagcagagccctctcccgcattccgggtaccaccactccttctcaggatggaaagcgctaccttcagcagggtttcgaggttcttgccaacggcctcaaggcgagcggctggaaggaggtcgattccctcaaggacagcgagcagaagaaccgcactttctcccacacctcatacatgtacatcaatggcgagcgtggcggtcctctagcgacttacctcgtcagcgccaagaagcgcagcaacttcaagctgtggctcaacaccgctgtcaagcgcgtcatccgtgagggcggccacattaccggtgtggaggttgaggccttccgcaacggcggctactccggaatcatccccgtcaccaacaccaccggccgcgtcgttctttccgccggcaccttcggcagcgccaagatccttctccgttccggcattggccccaaggaccagctcgaggtggtcaaggcctccgccgacggccctaccatggtcagcaactcgtcctggattgacctccccgtcggccacaacctggttgaccacaccaacaccgacaccgtcatccagcacaacaacgtgaccttctacgacttttacaaggcttgggacaaccccaacacgaccgacatgaacctgtacctcaatgggcgctccggcatcttcgcccaggccgcgcccaacattggccccttgttctgggaggagatcacgggcgccgacggcatcgtccgtcagctgcactggaccgcccgcgtcgagggcagcttcgagacccccgacggctacgccatgaccatgagccagtaccttggccgtggcgccacctcgcgcggccgcatgaccctcagccctaccctcaacaccgtcgtgtctgacctcccgtacctcaaggaccccaacgacaaggccgctgtcgttcagggtatcgtcaacctccagaaggctctcgccaacgtcaagggtctcacctgggcttaccctagcgccaaccagacggctgctgattttgttgacaagcaacccgtaacctaccaatcccgccgctccaaccactggatgggcaccaacaagatgggcaccgacgacggccgcagcggcggcaccgcagtcgtcgacaccaacacgcgcgtctatggcaccgacaacctgtacgtggtggacgcctcgattttccccggtgtgccgaccaccaaccctacc

SEQ ID NO: 74 - protein, 29 aa, Neurospora crassa
KWGWCGGPTYTGSQTCQAPYKCEKQNDWY

SEQ ID NO: 75 - DNA, 87 bp, Neurospora crassa
aagtggggctggtgcggcgggccgacgtatactggcagccagacgtgccaggcgccatataagtgcgagaagcagaatgattggtat

SEQ ID NO: 76 - protein, 188 aa, Neurospora crassa
QYTDPVNKITLSTWRPDPGSNSGGGDAATYAFGLVLPPDALTKDANEYIGLLRCDVGDAASPGWCGVSHGQSGQMTQSLLLMAWASKGQVFTSFRYASGYNVPGLYTGNATLTQISATVNSTQFELIYRCQDCFAWNQGGSKGSVSTSSGLLVLGRAAAKGNLQNPTCPDKAIPGFHDNGFGQYGAPL

SEQ ID NO: 77 - DNA, 564 bp, Neurospora crassa
caatataccgatcccgtgaacaagatcaccctcagcacctggcggccagaccctggttctaattctgggggtggagatgctgccacctacgcctttggcttggtcttgcctccggatgctctgaccaaagatgccaacgaatacatcggtctcttgcgctgtgatgttggtgatgcggcgagccccggatggtgtggtgtctcccacggccagtctggacaaatgacacagtcgttgttgctcatggcttgggcctccaagggtcaagtctttacctcatttcgctacgcatccggttataatgtgccaggactctacaccggaaatgcaaccctgacccagatctctgccactgtgaactcgacacagttcgaattgatctatcgctgccaggactgttttgcatggaaccaaggaggaagcaagggaagcgtatcaaccagcagtggccttctcgtcttgggccgtgccgcggccaagggaaatcttcagaacccgacttgccctgacaaggccattcccggctttcatgacaatgggtttggtcaatatggagcgcctctc

SEQ ID NO: 78 - protein, 539 aa, Neurospora crassa
APSKTYDYIIVGAGAGGIPIADKLSEAGKSVLLIEKGPPSTGRWKGTMKPEWLQGTNLTRFDVPGLCNQIWVDSAGIACTDTDQMAGCVLGGGTAVNAGLWWKPHPQDWNYNFPEGWKSRDTVPATNRVFGRIPGTWHPSQNGKLYRQEGFNVLASGLSKSGWKEVIPNDAYNQKNHTFGHSTFMFAKGERGGPLATYLVTAVARKQFTLWTNVAVRRAVRNGSRITGVELECLTDGGLSGTVNVTPNTGRVIFAAGTFGSAKLLLRSGIGPTDQLEIVKGSTDGPTFISKDQWINLPVGYNLMDHLNTDLIITHPDVVFYDFYEAWNTPIEGDKSAYLQNRSGILAQAAPNIGPLMWDELKGSDNIIRTLQWTARVEGSDQYTTSKHAMTLSQYLGRGVVSRGRMAISSGLDTNVAEHPYLHNDVDKQTVIQGIKNLQAALNVIPNLSWVLPPPNTTVESFINNMIVSPSNRRSNHWMGTAKLGKDDGRTGGSAVVDLNTKVYGTDNLFVVDASIFPGMTTGNPSAMIVIASEHAAQK

SEQ ID NO: 79 - DNA, 1617 bp, Neurospora crassa
gccccaagcaagacgtacgactacatcatcgttggcgccggtgctggtggcattcccattgcggacaagctcagcgaggccggaaaaagtgtgttgttgatcgaaaagggacctccctccactggaagatggaagggcaccatgaagcctgagtggcttcagggcacgaacttgactcgcttcgatgttcctggtctatgcaaccagatctgggtggactctgccggcatcgcctgtacagataccgaccaaatggcgggatgtgtcctgggcggaggaacggctgttaatgccggcctgtggtggaagccgcatcctcaggattggaactacaacttccccgagggctggaagtcgagagataccgtgccagccactaaccgtgtgttcggtcgcattcctggaacttggcatccttcgcaaaacggcaagctgtaccgacaagagggcttcaacgtcctagccagcgggctgagcaagagcggttggaaggaggtgatccccaacgatgcatacaaccagaagaaccacacctttggtcacagcaccttcatgttcgctaaaggcgagcgaggtggccctctggcaacataccttgtgacggcggtagctcgcaagcagttcactctctggaccaatgtagctgtgagaagggcagttcgtaacggaagccgtatcactggcgttgagctcgaatgcttgacggatggtggtctcagcggaactgtcaacgtgacccctaacactggccgtgttatctttgctgcaggcacttttggttccgccaagcttctccttcgcagcggtatcggacctaccgatcaactcgagattgtcaaggggtcgacggatggcccaacgttcatttccaaggaccaatggatcaaccttccagttggctacaacctcatggatcatctcaacactgatctcattatcacccatcctgacgttgtcttctacgacttctacgaggcttggaacacgcccattgaaggtgacaagagcgcctatcttcagaatagatctggaatccttgcccaggctgctcccaatattggtcctttgatgtgggatgaacttaagggctcggacaacatcattcgtactctgcaatggactgctcgagtggagggaagcgatcagtacaccacctctaagcatgccatgactctcagccaatatctcggcagaggtgttgtttccagaggccggatggcaatttcatcgggtctggacaccaatgtggccgagcacccgtacctccacaacgatgtcgacaagcagaccgtcatccaaggcatcaagaacctccaggcggcgctgaatgtcattcccaacctttcctgggttttgcctcccccgaacacgactgtcgagtcatttatcaacaatatgatcgtctcaccctccaatcgtcggtcaaaccattggatgggaactgccaagcttggcaaggacgatggccgtactggaggcagcgctgtcgtggatctgaacaccaaggtgtacggtaccgataacctctttgttgttgacgcctccatcttccctggtatgaccaccggcaacccgtcggcgatgatcgtgattgcctcggagcatgctgcacagaaa

SEQ ID NO: 80 - protein, 181 aa, Neurospora crassa
TFTDPDSGITFNTWGLAEDSPQTKGGFTFGVALPSDALTTDAKEFIGYLKCARNDESGWCGVSLGGPMTNSLLIAAWPHEDTVYTSLRFATGYAMPDVYQGDAEITQVSSSVNSTHFSLIFRCENCLQWSQSGATGGASTSNGVLVLGWVQAFADPGNPTCPDQITLEQHDNGMGIWGAQL

SEQ ID NO: 81 - DNA, 543 bp, Neurospora crassa
accttcaccgacccggactcgggcattaccttcaacacgtggggtctcgccgaggattctccccagactaagggcggtttcacttttggtgttgctctgccctctgatgccctcacgacagacgccaaggagttcatcggttacttgaaatgcgcgaggaacgatgagagcggttggtgcggtgtctccctgggcggccccatgaccaactcgctcctcatcgcggcctggccccacgaggacaccgtctacacctctctccgcttcgccaccggctatgccatgccggatgtctaccagggggacgccgagatcacccaggtctcctcctctgtcaactcgacgcacttcagcctcatcttcaggtgcgagaactgcctgcaatggagtcaaagcggcgccaccggcggtgcctccacctcgaacggcgtgttggtcctcggctgggtccaggcattcgccgaccccggcaacccgacctgccccgaccagatcaccctcgagcagcacgacaacggcatgggtatctggggtgcccagctc

SEQ ID NO: 82 - protein, 544 aa, Neurospora crassa
FDYIVVGGGAGGIPAADKLSEAGKSVLLIEKGFASTANTGGTLGPEWLEGHDLTRFDVPGLCNQIWVDSKGIACEDTDQMAGCVLGGGTAVNAGLWFKPYSLDWDYLFPSGWKYKDVQPAINRALSRIPGTDAPSTDGKRYYQQGFDVLSKGLAGGGWTSVTANNAPDKKNRTFSHAPFMFAGGERNGPLGTYFQTAKKRSNFKLWLNTSVKRVIRQGGHITGVEVEPFRDGGYQGIVPVTKVTGRVILSAGTFGSAKILLRSGIGPNDQLQVVAASEKDGPTMISNSSWINLPVGYNLDDHLNTDTVISHPDVVFYDFYEAWDNPIQSDKDSYLNSRTGILAQAAPNIGPMFWEEIKGADGIVRQLQWTARVEGSLGAPNGKTMTMSQYLGRGATSRGRMTITPSLTTVVSDVPYLKDPNDKEAVIQGIINLQNALKNVANLTWLFPNSTITPRQYVDSMVVSPSNRRSNHWMGTNKIGTDDGRKGGSAVVDLNTKVYGTDNLFVIDASIFPGVPTTNPTSYIVTASEHASARILALPDLTPV

SEQ ID NO: 83 - DNA, 1632 bp, Neurospora crassa
ttcgattacatcgtcgtgggcggcggtgccggtggcatccccgccgccgacaagctcagcgaggccggcaagagtgtgctgctcatcgagaagggctttgcctcgaccgccaacaccggaggcactctcggccccgagtggctcgagggccacgaccttacccgctttgacgtgccgggtctgtgcaaccagatctgggttgactccaaggggatcgcttgcgaggataccgaccagatggctggctgtgtcctcggcggcggtaccgccgtgaatgccggcctgtggttcaagccctactcgctcgactgggactacctcttccctagtggttggaagtacaaagacgtccagccggccatcaaccgcgccctctcgcgcatcccgggcaccgatgctccctcgaccgacggcaagcgctactaccaacagggcttcgacgtcctctccaagggcctggccggcggcggctggacctcggtcacggccaataacgcgccagacaagaagaaccgcaccttctcccatgcccccttcatgttcgccggcggcgagcgcaacggcccgctgggcacctacttccagaccgccaagaagcgcagcaacttcaagctctggctcaacacgtcggtcaagcgcgtcatccgccagggcggccacatcaccggcgtcgaggtcgagccgttccgcgacggcggttaccaaggcatcgtccccgtcaccaaggttacgggccgcgtcatcctctctgccggtacctttggcagtgcaaagatcctgctgaggagcggtatcggtccgaacgatcagctgcaggttgtcgcggcctcggagaaggatggccctaccatgatcagcaactcgtcctggatcaacctgcctgtcggctacaacctggatgaccacctcaacaccgacactgtcatctcccaccccgacgtcgtgttctacgacttctacgaggcgtgggacaatcccatccagtctgacaaggacagctacctcaactcgcgcacgggcatcctcgcccaagccgctcccaacattgggcctatgttctgggaagagatcaagggtgcggacggcattgttcgccagctccagtggactgcccgtgtcgagggcagcctgggtgcccccaacggcaagaccatgaccatgtcgcagtacctcggtcgtggtgccacctcgcgcggccgcatgaccatcaccccgtccctgacaactgtcgtctcggacgtgccctacctcaaggaccccaacgacaaggaggccgtcatccagggcatcatcaacctgcagaacgccctcaagaacgtcgccaacctgacctggctcttccccaactcgaccatcacgccgcgccaatacgttgacagcatggtcgtctccccgagcaaccggcgctccaaccactggatgggcaccaacaagatcggcaccgacgacgggcgcaagggcggctccgccgtcgtcgacctcaacaccaaggtctacggcaccgacaacctcttcgtcatcgacgcctccatcttccccggcgtgcccaccaccaaccccacctcgtacatcgtgacggcgtcggagcacgcctcggcccgcatcctcgccctgcccgacctcacgcccgtc

SEQ ID NO: 84 - protein, 34 aa, Neurospora crassa
PKYGQCGGREWSGSFVCADGSTCQMQNEWYSQCL

SEQ ID NO: 85 - DNA, 102 bp, Neurospora crassa
cccaagtacgggcagtgcggcggccgcgaatggagcggcagcttcgtctgcgccgacggctccacgtgccagatgcagaacgagtggtactcgcagtgcttg

SEQ ID NO: 86 - protein, 180 aa, Neurospora crassa
TYTDEATGIQFKTWTASEGAPFTFGLTLPADALEKDATEYIGLLRCQITDPASPSWCGISHGQSGQMTQALLLVAWASEDTVYTSFRYATGYTLPGLYTGDAKLTQISSSVSEDSFEVLFRCENCFSWDQDGTKGNVSTSNGNLVLGRAAAKDGVTGPTCPDTAEFGFHDNGFGQWGAVL

SEQ ID NO: 87 - DNA, 540 bp, Neurospora crassa
acctacaccgatgaggctaccggtatccaattcaagacgtggaccgcctccgagggcgcccctttcacgtttggcttgaccctccccgcggacgcgctggaaaaggatgccaccgagtacattggtctcctgcgttgccaaatcaccgatcccgcctcgcccagctggtgcggtatctcccacggccagtccggccagatgacgcaggcgctgctgctggtcgcctgggccagcgaggacaccgtctacacgtcgttccgctacgccaccggctacacgctccccggcctctacacgggcgacgccaagctgacccagatctcctcctcggtcagcgaggacagcttcgaggtgctgttccgctgcgaaaactgcttctcctgggaccaggatggcaccaagggcaacgtctcgaccagcaacggcaacctggtcctcggccgcgccgccgcgaaggatggtgtgacgggccccacgtgcccggacacggccgagttcggtttccatgataacggtttcggacagtggggtgccgtgctt

SEQ ID NO: 88 - protein, 541 aa, Neurospora crassa
APEDTYDYIVVGAGAGGITVADKLSEAGHKVLLIEKGPPSTGLWNGTMKPEWLESTDLTRFDVPGLCNQIWVDSAGIACTDTDQMAGCVLGGGTAVNAGLWWKPHPADWDENFPEGWKSSDLADATERVFKRIPGTSHPSQDGKLYRQEGFEVISKGLANAGWKEISANEAPSEKNHTYAHTEFMFSGGERGGPLATYLASAAERSNFNLWLNTAVRRAVRSGSKVTGVELECLTDGGFSGTVNLNEGGGVIFSAGAFGSAKLLLRSGIGPEDQLEIVASSKDGETFTPKDEWINLPVGHNLIDHLNTDLIITHPDVVFYDFYAAWDEPITEDKEAYLNSRSGILAQAAPNIGPMMWDQVTPSDGITRQFQWTCRVEGDSSKTNSTHAMTLSQYLGRGVVSRGRMGITSGLSTTVAEHPYLHNNGDLEAVIQGIQNVVDALSQVADLEWVLPPPDGTVADYVNSLIVSPANRRANHWMGTAKLGTDDGRSGGTSVVDLDTKVYGTDNLFVVDASVFPGMSTGNPSAMIVIVAEQAAQRILA

SEQ ID NO: 89 - DNA, 1623 bp, Neurospora crassa
gctcccgaggacacgtatgattacatcgttgtcggtgccggcgccggtggtatcaccgtcgccgacaagctcagcgaggccggccacaaggtccttctcatcgagaagggacccccttcgaccggcctgtggaacgggaccatgaagcccgagtggctcgagagcaccgaccttacccgcttcgacgttcccggcctgtgcaaccagatctgggtcgactctgccggcatcgcctgcaccgataccgaccagatggcgggctgcgttctcggcggtggcaccgctgtcaacgctggtttgtggtggaagccccaccccgctgactgggatgagaacttccccgaagggtggaagtcgagcgatctcgcggatgcgaccgagcgtgtcttcaagcgcatccccggcacgtcgcacccgtcgcaggacggcaagttgtaccgccaggagggcttcgaggtcatcagcaagggcctggccaacgccggctggaaggaaatcagcgccaacgaggcgcccagcgagaagaaccacacctatgcacacaccgagttcatgttctcgggcggtgagcgtggcggccccctggcgacgtaccttgcctcggctgccgagcgcagcaacttcaacctgtggctcaacactgccgtccggagggccgtccgcagcggcagcaaggtcaccggcgtcgagctcgagtgcctcacggacggtggcttcagcgggaccgtcaacctgaatgagggcggtggtgtcatcttctcggccggcgctttcggctcggccaagctgctccttcgcagcggtatcggtcctgaggaccagctcgagattgtggcgagctccaaggacggcgagaccttcactcccaaggacgagtggatcaacctccccgtcggccacaacctgatcgaccatctcaacactgacctcattatcacgcacccggatgtcgttttctatgacttctatgcggcctgggacgagcccatcacggaggataaggaggcctacctgaactcgcggtccggcattctcgcccaggcggcgcccaatatcggccctatgatgtgggatcaagtcacgccgtccgacggcatcacccgccagttccagtggacatgccgtgttgagggcgacagctccaagaccaactcgacccacgccatgaccctcagccagtacctcggccgtggcgtcgtctcgcgcggccggatgggcatcacctccgggctgagcacgacggtggccgagcacccgtacctgcacaacaacggcgacctggaggcggtcatccaggggatccagaacgtggtggacgcgctcagccaggtggccgacctcgagtgggtgctcccgccgcccgacgggacggtggccgactacgtcaacagcctgatcgtctcgccggccaaccgccgggccaaccactggatgggcacggccaagctgggcaccgacgacggccgctcgggcggcacctcggtcgtcgacctcgacaccaaggtgtacggcaccgacaacctgttcgtcgtcgacgcgtccgtcttccccggcatgtcgacgggcaacccgtcggccatgatcgtcatcgtggccgagcaggcggcgcagcgcatcctggcc

SEQ ID NO: 90 - protein, 326 aa, Neurospora crassa
MKLSVAAALSLAASEASAHYIFQQVGAGTSVNPVWKYIRKHTNYNSPVTDLTSKDLVCNVGASAEGVETLSVAAGSQVTFKTDTAVYHQGPTSVYLSKADGSLSDYDGSGGWFKIKDWGATFPGGEWTLSDTYTFTIPSCIPSGDYLLRIQQIGIHNPWPAGVPQFYLSCAHISVTGGGSASPATVSIPGAFKETDPGYTVNIYSNFNNYTVPGPEVFTCSGSGSGSGSGSGSGSTPPSQPTTSTTLPTSSTVVATTLKTSTVVATTKSSSSTTSSASSSGSQPTSPSGCTVAKYGQCGGIGYSGCTSCASGSTCKVGNDYYSQCL

SEQ ID NO: 91 - DNA, 981 bp, Neurospora crassa
atgaagctttcagttgctgccgccctttctctcgccgccagcgaggcctcggcccactacatcttccagcaagtcggcgccgggacctcggtcaacccggtttggaagtacatccgcaagcacaccaactacaactcgcccgtgaccgacttgacttccaaagaccttgtgtgcaacgtcggcgccagcgctgagggcgtcgaaaccctctccgttgctgccggctcccaggtcaccttcaagaccgacacggccgtctaccaccagggtcccacttccgtctacctctccaaggccgacgggtccctttccgactatgatggctcgggcggttggttcaagatcaaggactggggcgctaccttccccggtggtgaatggactttgtcggacacttacactttcacgatcccttcgtgtattccctcgggtgactaccttttgcgtattcagcagattggtatccacaacccctggcccgcaggtgttccccagttctacctctcctgcgctcacatttccgtgacgggcggtggtagcgcctcccccgccactgtctccatccctggagccttcaaggagaccgatcccggctacaccgtcaacatctactccaacttcaacaactacaccgtccccggccccgaggtattcacctgcagcggttctggcagcggttccggctccggctccggctccggctctacccccccatcccagccgaccacttctactaccctcccgacttcttcgaccgttgtcgcgaccaccctcaagacttcgactgtcgtcgccacgaccaagagcagcagcagcaccacttcgtcagcctcctcctcaggcagccagcccaccagcccttctggctgcacggtggccaagtacggacagtgcggtggcattggatacagcgggtgcacgagctgcgctagcgggtcgacctgcaaggttggcaatgactattactcgcagtgcttgtaa

SEQ ID NO: 92 - protein, 12 aa, Artificial Sequence
  Sequence Motif
HXXXXXXXXQXY